(19) United States
(12) Patent Application Publication
(10) Pub. No.: US 2024/0401151 A1
(43) Pub. Date: Dec. 5, 2024

(54) SINGLE NUCLEOTIDE POLYMORPHISM (SNP) MOLECULAR MARKER COMBINATION FOR GENETIC RELATIONSHIP IDENTIFICATION OF HUAXI CATTLE, USE, AND IDENTIFICATION METHOD

(71) Applicant: Institute of Animal Sciences of CAAS, Beijing (CN)

(72) Inventors: Xue GAO, Beijing (CN); Bo ZHU, Beijing (CN); Junya LI, Beijing (CN); Yuanqing WANG, Beijing (CN); Huijiang GAO, Beijing (CN); Yan CHEN, Beijing (CN); Lupei ZHANG, Beijing (CN); Zezhao WANG, Beijing (CN)

(21) Appl. No.: 18/398,681

(22) Filed: Dec. 28, 2023

(30) Foreign Application Priority Data
Jun. 2, 2023 (CN) 202310653710.3

(51) Int. Cl. C12Q 1/6888

(52) U.S. Cl. CPC C12Q 1/6888; C12Q 2600/156

(57) ABSTRACT

The present disclosure provides a single nucleotide polymorphism (SNP) molecular marker combination for genetic relationship identification of a Huaxi cattle, use, and an identification method, and belongs to the technical field of genotyping. The present disclosure provides a SNP molecular marker combination for genetic relationship identification of a Huaxi cattle, where the genetic relationship identification of the Huaxi cattle is completed with at least 70 of 1,000 sites. In the present disclosure, the 1,000 sites are the SNP molecular markers distributed on 29 autosomes, and two adjacent SNP molecular markers on a same autosome have an average distance of 2 Mb. A minimum allele frequency (MAF), an expected heterozygosity (HExp), and a polymorphism information content (PIC) of the SNP molecular marker combination have average values of 0.4846, 0.4983, and 0.3741, respectively; and when a maternal genotype is unknown, there is a cumulative probability of exclusion (CPE) of 0.9999999999999.

### TECHNICAL FIELD

The present disclosure belongs to the technical field of genotyping, and specifically relates to a single nucleotide polymorphism (SNP) molecular marker combination for genetic relationship identification of a Huaxi cattle, use, and an identification method.

### BACKGROUND

The accuracy of pedigrees is critical to cattle breeding. Incorrect pedigrees can greatly reduce the accuracy of genetic evaluation, affect the selection effect, and hinder the genetic improvement of a population, thus causing a huge economic impact on the breeding industry. However, pedigree records generally have errors due to various factors, such as breeding records, calving records, and human errors in actual breeding production. Especially in extensive free-range pastures, it is difficult to determine the male parent or even both parents of a calf, thereby leading to pedigree errors. Sire identification was replaced for 11% of Holstein cows that were sired by AI bulls and had records in the US database for national genetic evaluations; US evaluations were computed based on those modified pedigrees and compared with official national evaluations^{[1]}. Due to different feeding methods and production management conditions in different countries, there are certain differences in pedigree error rates, but pedigree errors are difficult to be completely avoided during actual production and management. Research by Guo Gang et al.^{[2]} has showed that the Holstein cows in Beijing have an average pedigree error rate of 20.9%. The average pedigree error rate of 11% in the world may lead to lower estimates of inbreeding coefficients, male animal variance, and cross-national genetic correlations in actual production. Estimated breeding values for the populations with 10% incorrect paternity were biased, especially in the later generations. Genetic gains were 4.3% higher with correct paternity identification at a heritability of 0.25 within 20 yr^{[3]}. In the genetic evaluation of dairy cows, a population genetic progress may be reduced by 11% to 15% if the cow has an error rate of male parent recording at 11%. Instituting a system of quality control, especially at the level of the inseminator, should reduce paternity errors to no more than 8%, and increase genetic progress by at least 1%^{[4]}. Accordingly, a complete and accurate pedigree is extremely necessary for the production and development of the entire cattle industry.

Huaxi cattle are a new breed of beef cattle developed by the Institute of Animal Science (IAS) of Chinese Academy of Agricultural Sciences (CAAS) after more than 40 yr of painstaking efforts. The breed was approved by the National Livestock and Poultry Genetic Resources Committee in 2021 and acquired the Certificate of National Livestock and Poultry New Breed. The Huaxi cattle show rapid growth, high feed conversion rate and meat clean rate, desirable meat production and reproductive performance, and strong stress resistance. Compared with similar international beef cattle breeds, the daily weight gain, slaughter rate, and clean meat rate of Huaxi cattle are at an internationally advanced level. Huaxi cattle are adapted to pastoral areas, agricultural areas, and the agricultural-pastoral ecotone in the north, as well as grassy mountains and grassy slopes in the south in China. Since the breeding methods in pastoral areas are mostly extensive, both bulls and artificial insemination are adopted during the breeding. In addition, human errors occur during the implementation of artificial insemination may lead to an extremely high pedigree error rate. As a result, it is highly important to confirm the genetic relationship to accurately estimate an individual breeding value of the Huaxi cattle and to accelerate genetic progress.

At present, the commonly-used molecular methods for paternity testing mainly include DNA fingerprinting, microsatellites, and SNP molecular markers. Although the molecular markers used in the past few decades have been gradually updated from allozymes and the microsatellites (also known as short tandem repeats, STRs) to SNPs, the theoretical basis of paternity analysis has not deviated from the requirements of following Mendel's law of inheritance. Single nucleotide polymorphism (SNP) markers refer to DNA sequence polymorphisms caused by variations in a single base at the genome level, are generally caused by transition or transversion of the single base, or caused by insertion or deletion of bases. However, the transition or transversion shows a probability of occurrence that is twice as high as that of the insertion or deletion. Generally speaking, the SNP markers have a minimum allele frequency (MAF) of not less than 1% in a population, but there are also cases where the MAF is less than 1% (such as cDNA).

There are currently no reports on genetic relationship identification of Huaxi cattle using SNP markers. The lack of pedigree records creates great difficulties in promoting the quality traceability system of Huaxi cattle; moreover, the application of pedigree information is extremely important in the genetic research of Huaxi cattle. In view of this, a complete and accurate pedigree is highly necessary for the development and promotion of the entire breeding industry of Huaxi cattle. In the case where the accuracy of actual records cannot be guaranteed, the genetic relationship identification has become an important link in the genetic breeding and improvement of Huaxi cattle.

### CITED REFERENCES

[1]. Banos G, Wiggans G R, Powell R L. Impact of paternity errors in cow identification on genetic evaluations and international comparisons [J]. Journal of dairy science, 2001, 84(11): 2523-2529.
[2]. GUO Gang, ZHOU Lei, LIU Lin, LI Dong, ZHANG Shengli, LIU Jianfeng, DING Xiangdong, ZHANG Yi, WANG Yachun, ZHANG Qin, and ZHANG Yuan. Parentage Inference with Single Nucleotide Polymorphism Markers in the Chinese Holstein in Beijing [J]. Acta Veterinaria et Zootechnica Sinica, 2012, 43(01): 44-49.
[3]. Israel C, Weller J I. Effect of misidentification on genetic gain and estimation of breeding value in dairy cattle populations [J]. Journal of Dairy Science, 2000, 83(1): 181-187.
[4]. Weller J I, Feldmesser E, Golik M, et al. Factors affecting incorrect paternity assignment in the Israeli Holstein population [J]. Journal of Dairy Science, 2004, 87(8): 2627-2640.

### SUMMARY

A purpose of the present disclosure is to provide a single nucleotide polymorphism (SNP) molecular marker combination for genetic relationship identification of a Huaxi cattle, use, and an identification method. The SNP molecular marker combination can be used for complete and accurate pedigree identification of the Huaxi cattle, thus improving an accuracy of Huaxi cattle breeding.

The present disclosure provides an SNP molecular marker combination for genetic relationship identification of a Huaxi cattle, including at least 70 of SNP molecular markers shown in Table 1.

Preferably, Bos_taurus_UMD_3.1 serves as a reference genome for a site of each of the SNP molecular markers.

The present disclosure further provides a probe for identifying the SNP molecular marker combination.

The present disclosure further provides a gene chip prepared based on the SNP molecular marker combination.

Preferably, the gene chip includes a liquid chip.

The present disclosure further provides use of the SNP molecular marker combination or the probe or the gene chip in genetic relationship identification of a Huaxi cattle.

The present disclosure further provides use of the SNP molecular marker combination or the probe or the gene chip in improved genetic breeding of a Huaxi cattle.

The present disclosure further provides a method for genetic relationship identification of a Huaxi cattle, including the following steps: identifying a genotype of the SNP molecular marker combination using a genomic DNA of a Huaxi cattle to be identified as a template; calculating an logarithm of the odds (LOD) value of a paternity index according to the genotype of the Huaxi cattle to be identified; and identifying a genetic relationship of the Huaxi cattle to be identified based on the LOD value.

Preferably, when the LOD value is greater than 0, a candidate parent has a possibility of being a true parent, and an individual with a maximum LOD value is regarded as a most similar parent; and when the LOD value is less than 0, the candidate parent does not have the possibility of being the true parent.

Beneficial effects: the present disclosure provides a SNP molecular marker combination for genetic relationship identification of a Huaxi cattle, where multiple SNP sites on 29 autosomes were selected through data analysis of a sequencing result of 1,252 Huaxi cattle; and the genetic relationship identification of the Huaxi cattle is completed with at least 70 of 1,000 sites. In the present disclosure, the 1,000 SNP molecular markers are distributed on the 29 autosomes, and two adjacent SNP molecular markers on a same autosome have an average distance of 2 Mb. A minimum allele frequency (MAF), an expected heterozygosity (HExp), and a polymorphism information content (PIC) of the SNP molecular marker combination have average values of 0.4846, 0.4983, and 0.3741, respectively; and when a maternal genotype is unknown, there is a cumulative probability of exclusion (CPE) of 0.9999999999999. The SNP molecular marker combination can be used for complete and accurate pedigree identification of the Huaxi cattle, thereby improving a breeding accuracy and accelerating a genetic progress of the Huaxi cattle, and exhibit excellent application prospects and economic benefits.

### BRIEF DESCRIPTION OF THE DRAWINGS

FIG. 1 shows specific operational steps of the method for genetic relationship identification of a Huaxi cattle.

### DETAILED DESCRIPTION OF THE EMBODIMENTS

The present disclosure provides an SNP molecular marker combination for genetic relationship identification of a Huaxi cattle, including at least 70 of SNP molecular markers shown in Table 1.

In the present disclosure, sequencing data results of 1,252 Huaxi cattle were analyzed and selected to identify 1,000 SNP sites on 29 autosomes, which can be used in the SNP molecular marker combination for genetic relationship identification of a Huaxi cattle population. The 1,000 SNP molecular markers are distributed on the 29 autosomes, and two adjacent SNP molecular markers on a same autosome have an average distance of 2 Mb. Among the SNP molecular marker information in Table 1, Bos_taurus_UMD_3.1 serves as a reference genome for a site. The SNP molecular marker combination includes 1,000 SNP sites. The MAF, HExp, and PIC of the SNP molecular marker combination have average values of 0.4846, 0.4983, and 0.3741, respectively; and the 1,000 SNP molecular marker combination has a CPE of 0.9999999999999, which makes paternity testing extremely efficient.

Table 1 Information on 1,000 SNP molecular markers

| | | | Base | | | | Probability of |
|---|---|---|---|---|---|---|---|
| SNP name | Chromosome | Position | variation | MAF | HExp | PIC | exclusion (PE) |
| chr1: 146736 | 1 | 146736 | C/T | 0.4996 | 0.5 | 0.375 | 0.125 |
| BovineHD0100001067 | 1 | 3232948 | G/A | 0.4992 | 0.5 | 0.375 | 0.125 |
| BovineHD0100001535 | 1 | 4740225 | C/A | 0.4976 | 0.5 | 0.375 | 0.125 |
| BovineHD0100002084 | 1 | 6397764 | G/A | 0.4984 | 0.5 | 0.375 | 0.125 |
| chr1: 8222595 | 1 | 8222595 | C/T | 0.4976 | 0.5 | 0.375 | 0.125 |
| chr1: 10268092 | 1 | 10268092 | T/G | 0.4669 | 0.498 | 0.374 | 0.124 |
| chr1: 12788006 | 1 | 12788006 | C/T | 0.488 | 0.5 | 0.375 | 0.125 |
| chr1: 14370430 | 1 | 14370430 | C/T | 0.4677 | 0.498 | 0.374 | 0.124 |
| chr1: 17929625 | 1 | 17929625 | A/C | 0.496 | 0.5 | 0.375 | 0.125 |
| Hapmap51222-BTA-39456 | 1 | 19805218 | T/C | 0.4141 | 0.485 | 0.368 | 0.118 |
| chr1: 20746789 | 1 | 20746789 | T/C | 0.494 | 0.5 | 0.375 | 0.125 |
| chr1: 22937855 | 1 | 22937855 | G/A | 0.4928 | 0.5 | 0.375 | 0.125 |
| chr1: 24948004 | 1 | 24948004 | G/A | 0.4952 | 0.5 | 0.375 | 0.125 |
| chr1: 27982761 | 1 | 27982761 | T/C | 0.4509 | 0.495 | 0.373 | 0.123 |
| BovineHD0100008442 | 1 | 28450059 | G/A | 0.4101 | 0.484 | 0.367 | 0.117 |
| chr1: 30343940 | 1 | 30343940 | C/T | 0.4776 | 0.499 | 0.374 | 0.125 |
| chr1: 31151169 | 1 | 31151169 | T/C | 0.4976 | 0.5 | 0.375 | 0.125 |
| BovineHD0100009944 | 1 | 34665393 | A/G | 0.498 | 0.5 | 0.375 | 0.125 |
| chr1: 36240622 | 1 | 36240622 | T/C | 0.4149 | 0.486 | 0.368 | 0.118 |
| chr1: 38821576 | 1 | 38821576 | G/C | 0.4992 | 0.5 | 0.375 | 0.125 |
| chr1: 41190611 | 1 | 41190611 | A/G | 0.4952 | 0.5 | 0.375 | 0.125 |
| chr1: 43473529 | 1 | 43473529 | C/G | 0.4984 | 0.5 | 0.375 | 0.125 |
| chr1: 46242711 | 1 | 46242711 | A/G | 0.4992 | 0.5 | 0.375 | 0.125 |
| BovineHD0100013967 | 1 | 49559687 | T/G | 0.5 | 0.5 | 0.375 | 0.125 |
| chr1: 51894494 | 1 | 51894494 | C/A | 0.4956 | 0.5 | 0.375 | 0.125 |
| chr1: 53589018 | 1 | 53589018 | A/G | 0.4992 | 0.5 | 0.375 | 0.125 |
| BovineHD0100015794 | 1 | 56127149 | A/G | 0.4908 | 0.5 | 0.375 | 0.125 |
| BovineHD0100016547 | 1 | 58465963 | G/A | 0.4992 | 0.5 | 0.375 | 0.125 |
| Hapmap48290-BTA-31675 | 1 | 61069157 | A/G | 0.4968 | 0.5 | 0.375 | 0.125 |
| chr1: 63123889 | 1 | 63123889 | C/T | 0.4876 | 0.5 | 0.375 | 0.125 |
| chr1: 65202434 | 1 | 65202434 | C/T | 0.4764 | 0.499 | 0.374 | 0.124 |
| chr1: 67249619 | 1 | 67249619 | C/T | 0.4944 | 0.5 | 0.375 | 0.125 |
| chr1: 69353459 | 1 | 69353459 | T/C | 0.4944 | 0.5 | 0.375 | 0.125 |
| chr1: 71116985 | 1 | 71116985 | G/A | 0.474 | 0.499 | 0.374 | 0.124 |
| chr1: 73343620 | 1 | 73343620 | T/C | 0.4976 | 0.5 | 0.375 | 0.125 |
| chr1: 75567962 | 1 | 75567962 | C/T | 0.3954 | 0.478 | 0.364 | 0.114 |
| chr1: 77468186 | 1 | 77468186 | C/T | 0.5 | 0.5 | 0.375 | 0.125 |
| chr1: 79185721 | 1 | 79185721 | T/C | 0.4828 | 0.5 | 0.375 | 0.125 |
| chr1: 81072008 | 1 | 81072008 | T/C | 0.4117 | 0.485 | 0.367 | 0.117 |
| chr1: 83093568 | 1 | 83093568 | T/C | 0.4976 | 0.5 | 0.375 | 0.125 |
| chr1: 85247068 | 1 | 85247068 | C/A | 0.3674 | 0.465 | 0.357 | 0.108 |
| chr1: 87604486 | 1 | 87604486 | A/G | 0.4976 | 0.5 | 0.375 | 0.125 |
| BovineHD0100025718 | 1 | 90391338 | T/G | 0.4617 | 0.497 | 0.374 | 0.124 |
| chr1: 92106250 | 1 | 92106250 | C/T | 0.4996 | 0.5 | 0.375 | 0.125 |
| chr1: 94141904 | 1 | 94141904 | T/C | 0.4852 | 0.5 | 0.375 | 0.125 |
| chr1: 96618535 | 1 | 96618535 | A/T | 0.4992 | 0.5 | 0.375 | 0.125 |
| chr1: 98213895 | 1 | 98213895 | G/A | 0.4976 | 0.5 | 0.375 | 0.125 |
| chr1: 102597974 | 1 | 102597974 | A/T | 0.4792 | 0.499 | 0.375 | 0.125 |
| chr1: 104553440 | 1 | 104553440 | C/A | 0.5 | 0.5 | 0.375 | 0.125 |
| chr1: 106360058 | 1 | 106360058 | A/G | 0.4948 | 0.5 | 0.375 | 0.125 |
| chr1: 108109512 | 1 | 108109512 | A/G | 0.5 | 0.5 | 0.375 | 0.125 |
| chr1: 111219204 | 1 | 111219204 | G/T | 0.4968 | 0.5 | 0.375 | 0.125 |
| chr1: 113316161 | 1 | 113316161 | A/G | 0.4996 | 0.5 | 0.375 | 0.125 |
| chr1: 115307210 | 1 | 115307210 | C/T | 0.4996 | 0.5 | 0.375 | 0.125 |
| chr1: 117232512 | 1 | 117232512 | G/A | 0.4988 | 0.5 | 0.375 | 0.125 |
| chr1: 119142301 | 1 | 119142301 | A/G | 0.4996 | 0.5 | 0.375 | 0.125 |
| chr1: 121619169 | 1 | 121619169 | C/G | 0.498 | 0.5 | 0.375 | 0.125 |
| chr1: 123306221 | 1 | 123306221 | T/C | 0.4569 | 0.496 | 0.373 | 0.123 |
| chr1: 125963762 | 1 | 125963762 | T/C | 0.4992 | 0.5 | 0.375 | 0.125 |
| chr1: 127473224 | 1 | 127473224 | C/T | 0.3774 | 0.47 | 0.36 | 0.11 |
| chr1: 129312196 | 1 | 129312196 | T/C | 0.4872 | 0.5 | 0.375 | 0.125 |
| chr1: 131338591 | 1 | 131338591 | C/T | 0.4936 | 0.5 | 0.375 | 0.125 |
| chr1: 133231492 | 1 | 133231492 | C/T | 0.4613 | 0.497 | 0.373 | 0.124 |
| chr1: 135638938 | 1 | 135638938 | G/A | 0.4984 | 0.5 | 0.375 | 0.125 |
| chr1: 137647060 | 1 | 137647060 | G/T | 0.4976 | 0.5 | 0.375 | 0.125 |
| chr1: 139172793 | 1 | 139172793 | T/G | 0.4956 | 0.5 | 0.375 | 0.125 |
| chr1: 141796859 | 1 | 141796859 | C/G | 0.4537 | 0.496 | 0.373 | 0.123 |
| BovineHD0100041816 | 1 | 144840991 | G/A | 0.4988 | 0.5 | 0.375 | 0.125 |
| chr1: 147365032 | 1 | 147365032 | A/G | 0.492 | 0.5 | 0.375 | 0.125 |
| chr1: 149696295 | 1 | 149696295 | G/T | 0.4768 | 0.499 | 0.374 | 0.124 |
| chr1: 151931941 | 1 | 151931941 | C/T | 0.4968 | 0.5 | 0.375 | 0.125 |
| chr1: 153033656 | 1 | 153033656 | C/T | 0.4868 | 0.5 | 0.375 | 0.125 |
| chr1: 157013585 | 1 | 157013585 | A/G | 0.494 | 0.5 | 0.375 | 0.125 |
| chr2: 2292914 | 2 | 2292914 | C/T | 0.498 | 0.5 | 0.375 | 0.125 |
| chr2: 3201445 | 2 | 3201445 | T/G | 0.4481 | 0.495 | 0.372 | 0.122 |
| chr2: 6547325 | 2 | 6547325 | C/G | 0.4924 | 0.5 | 0.375 | 0.125 |
| BovineHD0200002751 | 2 | 9691620 | C/T | 0.4629 | 0.497 | 0.374 | 0.124 |
| chr2: 10515607 | 2 | 10515607 | G/A | 0.4984 | 0.5 | 0.375 | 0.125 |
| BovineHD0200003077 | 2 | 11166355 | T/G | 0.4812 | 0.499 | 0.375 | 0.125 |
| chr2: 12469133 | 2 | 12469133 | A/G | 0.4924 | 0.5 | 0.375 | 0.125 |
| chr2: 14337459 | 2 | 14337459 | C/A | 0.4988 | 0.5 | 0.375 | 0.125 |
| chr2: 16361378 | 2 | 16361378 | T/C | 0.4752 | 0.499 | 0.374 | 0.124 |
| chr2: 18736141 | 2 | 18736141 | G/A | 0.4832 | 0.5 | 0.375 | 0.125 |
| chr2: 20899037 | 2 | 20899037 | G/A | 0.4956 | 0.5 | 0.375 | 0.125 |
| chr2: 23048877 | 2 | 23048877 | A/C | 0.4992 | 0.5 | 0.375 | 0.125 |
| chr2: 25766917 | 2 | 25766917 | A/G | 0.4069 | 0.483 | 0.366 | 0.116 |
| chr2: 27704806 | 2 | 27704806 | C/A | 0.4896 | 0.5 | 0.375 | 0.125 |
| chr2: 29923519 | 2 | 29923519 | C/A | 0.4653 | 0.498 | 0.374 | 0.124 |
| chr2: 31692834 | 2 | 31692834 | T/G | 0.4992 | 0.5 | 0.375 | 0.125 |
| chr2: 33882604 | 2 | 33882604 | G/A | 0.4153 | 0.486 | 0.368 | 0.118 |
| chr2: 35706579 | 2 | 35706579 | C/G | 0.4884 | 0.5 | 0.375 | 0.125 |
| chr2: 38317092 | 2 | 38317092 | G/A | 0.494 | 0.5 | 0.375 | 0.125 |
| chr2: 40402873 | 2 | 40402873 | G/A | 0.4988 | 0.5 | 0.375 | 0.125 |
| chr2: 42785850 | 2 | 42785850 | A/C | 0.4832 | 0.5 | 0.375 | 0.125 |
| chr2: 45298638 | 2 | 45298638 | T/C | 0.4952 | 0.5 | 0.375 | 0.125 |
| chr2: 47490334 | 2 | 47490334 | C/G | 0.4924 | 0.5 | 0.375 | 0.125 |
| chr2: 50246865 | 2 | 50246865 | A/T | 0.4996 | 0.5 | 0.375 | 0.125 |
| chr2: 53125067 | 2 | 53125067 | T/G | 0.4704 | 0.498 | 0.374 | 0.124 |
| chr2: 55611799 | 2 | 55611799 | A/T | 0.4972 | 0.5 | 0.375 | 0.125 |
| chr2: 57700577 | 2 | 57700577 | A/C | 0.4904 | 0.5 | 0.375 | 0.125 |
| BovineHD0200018019 | 2 | 62411616 | C/T | 0.4964 | 0.5 | 0.375 | 0.125 |
| chr2: 63275266 | 2 | 63275266 | T/A | 0.4988 | 0.5 | 0.375 | 0.125 |
| BovineHD0200018552 | 2 | 64242565 | A/G | 0.478 | 0.499 | 0.375 | 0.125 |
| chr2: 68158765 | 2 | 68158765 | G/C | 0.4912 | 0.5 | 0.375 | 0.125 |
| chr2: 70740356 | 2 | 70740356 | C/G | 0.4984 | 0.5 | 0.375 | 0.125 |
| chr2: 73566014 | 2 | 73566014 | T/C | 0.4421 | 0.493 | 0.372 | 0.122 |
| chr2: 75948236 | 2 | 75948236 | G/A | 0.4968 | 0.5 | 0.375 | 0.125 |
| BovineHD0200022742 | 2 | 79151817 | G/A | 0.4996 | 0.5 | 0.375 | 0.125 |
| chr2: 81673931 | 2 | 81673931 | G/A | 0.3966 | 0.479 | 0.364 | 0.115 |
| chr2: 83323019 | 2 | 83323019 | T/C | 0.4421 | 0.493 | 0.372 | 0.122 |
| chr2: 86824344 | 2 | 86824344 | G/A | 0.4964 | 0.5 | 0.375 | 0.125 |
| chr2: 89335404 | 2 | 89335404 | T/C | 0.5 | 0.5 | 0.375 | 0.125 |
| chr2: 91648591 | 2 | 91648591 | G/A | 0.498 | 0.5 | 0.375 | 0.125 |
| BovineHD0200027563 | 2 | 95935954 | T/C | 0.498 | 0.5 | 0.375 | 0.125 |
| chr2: 97928774 | 2 | 97928774 | T/C | 0.4928 | 0.5 | 0.375 | 0.125 |
| chr2: 99749502 | 2 | 99749502 | C/T | 0.4261 | 0.489 | 0.369 | 0.12 |
| chr2: 102483918 | 2 | 102483918 | C/G | 0.4022 | 0.481 | 0.365 | 0.116 |
| BovineHD0200030211 | 2 | 105005873 | C/T | 0.4844 | 0.5 | 0.375 | 0.125 |
| chr2: 107473778 | 2 | 107473778 | G/A | 0.4724 | 0.499 | 0.374 | 0.124 |
| chr2: 109312516 | 2 | 109312516 | T/C | 0.4936 | 0.5 | 0.375 | 0.125 |
| chr2: 111984199 | 2 | 111984199 | A/G | 0.4844 | 0.5 | 0.375 | 0.125 |
| chr2: 114011690 | 2 | 114011690 | T/G | 0.4996 | 0.5 | 0.375 | 0.125 |
| chr2: 116006681 | 2 | 116006681 | C/G | 0.4896 | 0.5 | 0.375 | 0.125 |
| chr2: 118519219 | 2 | 118519219 | C/T | 0.3522 | 0.457 | 0.352 | 0.104 |
| chr2: 121231584 | 2 | 121231584 | C/G | 0.4453 | 0.494 | 0.372 | 0.122 |
| chr2: 123977838 | 2 | 123977838 | A/G | 0.4784 | 0.499 | 0.375 | 0.125 |
| chr2: 125154883 | 2 | 125154883 | T/C | 0.4972 | 0.5 | 0.375 | 0.125 |
| chr2: 127550880 | 2 | 127550880 | C/T | 0.4157 | 0.486 | 0.368 | 0.118 |
| chr2: 129689230 | 2 | 129689230 | C/A | 0.4992 | 0.5 | 0.375 | 0.125 |
| chr2: 131912580 | 2 | 131912580 | G/A | 0.5 | 0.5 | 0.375 | 0.125 |
| chr2: 133126495 | 2 | 133126495 | A/G | 0.4844 | 0.5 | 0.375 | 0.125 |
| chr2: 135007150 | 2 | 135007150 | A/C | 0.47 | 0.498 | 0.374 | 0.124 |
| chr2: 136693511 | 2 | 136693511 | A/T | 0.4988 | 0.5 | 0.375 | 0.125 |
| BovineHD0300000204 | 3 | 922000 | A/C | 0.4892 | 0.5 | 0.375 | 0.125 |
| BovineHD0300000351 | 3 | 1296645 | T/C | 0.4996 | 0.5 | 0.375 | 0.125 |
| BovineHD0300000547 | 3 | 2038037 | C/T | 0.4796 | 0.499 | 0.375 | 0.125 |
| chr3: 2972559 | 3 | 2972559 | G/A | 0.4856 | 0.5 | 0.375 | 0.125 |
| chr3: 4609284 | 3 | 4609284 | T/A | 0.4205 | 0.488 | 0.369 | 0.119 |
| chr3: 6714473 | 3 | 6714473 | G/A | 0.4241 | 0.489 | 0.369 | 0.119 |
| chr3: 8163083 | 3 | 8163083 | C/A | 0.4988 | 0.5 | 0.375 | 0.125 |
| chr3: 10889683 | 3 | 10889683 | T/C | 0.4996 | 0.5 | 0.375 | 0.125 |
| chr3: 12700815 | 3 | 12700815 | T/G | 0.4992 | 0.5 | 0.375 | 0.125 |
| chr3: 14888178 | 3 | 14888178 | A/T | 0.4685 | 0.498 | 0.374 | 0.124 |
| chr3: 16774367 | 3 | 16774367 | C/G | 0.4988 | 0.5 | 0.375 | 0.125 |
| chr3: 18214907 | 3 | 18214907 | G/A | 0.4856 | 0.5 | 0.375 | 0.125 |
| chr3: 20227589 | 3 | 20227589 | A/T | 0.4956 | 0.5 | 0.375 | 0.125 |
| chr3: 22888010 | 3 | 22888010 | T/C | 0.4908 | 0.5 | 0.375 | 0.125 |
| chr3: 24121607 | 3 | 24121607 | G/A | 0.4916 | 0.5 | 0.375 | 0.125 |
| chr3: 26062421 | 3 | 26062421 | A/C | 0.5 | 0.5 | 0.375 | 0.125 |
| chr3: 28243923 | 3 | 28243923 | T/A | 0.4988 | 0.5 | 0.375 | 0.125 |
| chr3: 31210779 | 3 | 31210779 | C/T | 0.4996 | 0.5 | 0.375 | 0.125 |
| chr3: 33410492 | 3 | 33410492 | A/C | 0.4976 | 0.5 | 0.375 | 0.125 |
| chr3: 35703444 | 3 | 35703444 | A/C | 0.4912 | 0.5 | 0.375 | 0.125 |
| chr3: 37862314 | 3 | 37862314 | T/C | 0.4848 | 0.5 | 0.375 | 0.125 |
| chr3: 40047483 | 3 | 40047483 | C/A | 0.4976 | 0.5 | 0.375 | 0.125 |
| chr3: 42347013 | 3 | 42347013 | A/G | 0.4992 | 0.5 | 0.375 | 0.125 |
| chr3: 46141906 | 3 | 46141906 | T/C | 0.49 | 0.5 | 0.375 | 0.125 |
| chr3: 48531987 | 3 | 48531987 | C/T | 0.4888 | 0.5 | 0.375 | 0.125 |
| chr3: 50087938 | 3 | 50087938 | C/A | 0.4908 | 0.5 | 0.375 | 0.125 |
| chr3: 52363329 | 3 | 52363329 | G/C | 0.3558 | 0.459 | 0.353 | 0.105 |
| chr3: 55465323 | 3 | 55465323 | A/G | 0.4896 | 0.5 | 0.375 | 0.125 |
| chr3: 58278551 | 3 | 58278551 | T/C | 0.4948 | 0.5 | 0.375 | 0.125 |
| chr3: 60872835 | 3 | 60872835 | C/T | 0.4932 | 0.5 | 0.375 | 0.125 |
| chr3: 63972726 | 3 | 63972726 | A/G | 0.4776 | 0.499 | 0.374 | 0.125 |
| chr3: 65931583 | 3 | 65931583 | G/A | 0.4984 | 0.5 | 0.375 | 0.125 |
| chr3: 68033961 | 3 | 68033961 | G/A | 0.496 | 0.5 | 0.375 | 0.125 |
| chr3: 71381417 | 3 | 71381417 | T/C | 0.4748 | 0.499 | 0.374 | 0.124 |
| chr3: 73731721 | 3 | 73731721 | T/C | 0.4149 | 0.486 | 0.368 | 0.118 |
| chr3: 75720895 | 3 | 75720895 | T/A | 0.492 | 0.5 | 0.375 | 0.125 |
| chr3: 78450019 | 3 | 78450019 | C/A | 0.4936 | 0.5 | 0.375 | 0.125 |
| chr3: 80270744 | 3 | 80270744 | C/T | 0.4377 | 0.492 | 0.371 | 0.121 |
| chr3: 83162493 | 3 | 83162493 | T/C | 0.4992 | 0.5 | 0.375 | 0.125 |
| chr3: 85988901 | 3 | 85988901 | T/C | 0.4892 | 0.5 | 0.375 | 0.125 |
| chr3: 87656169 | 3 | 87656169 | G/A | 0.4976 | 0.5 | 0.375 | 0.125 |
| chr3: 89847174 | 3 | 89847174 | G/T | 0.4984 | 0.5 | 0.375 | 0.125 |
| chr3: 91558792 | 3 | 91558792 | C/A | 0.4968 | 0.5 | 0.375 | 0.125 |
| chr3: 93499656 | 3 | 93499656 | T/C | 0.498 | 0.5 | 0.375 | 0.125 |
| chr3: 95012162 | 3 | 95012162 | A/T | 0.498 | 0.5 | 0.375 | 0.125 |
| chr3: 97148386 | 3 | 97148386 | T/G | 0.4984 | 0.5 | 0.375 | 0.125 |
| chr3: 100912209 | 3 | 100912209 | T/C | 0.4281 | 0.49 | 0.37 | 0.12 |
| chr3: 102515238 | 3 | 102515238 | A/G | 0.5 | 0.5 | 0.375 | 0.125 |
| chr3: 104164934 | 3 | 104164934 | C/T | 0.4964 | 0.5 | 0.375 | 0.125 |
| chr3: 106219168 | 3 | 106219168 | G/C | 0.4996 | 0.5 | 0.375 | 0.125 |
| chr3: 108180618 | 3 | 108180618 | G/C | 0.4728 | 0.499 | 0.374 | 0.124 |
| BovineHD4100002462 | 3 | 110010745 | T/C | 0.4976 | 0.5 | 0.375 | 0.125 |
| chr3: 112430477 | 3 | 112430477 | C/T | 0.4992 | 0.5 | 0.375 | 0.125 |
| chr3: 114619239 | 3 | 114619239 | G/A | 0.4916 | 0.5 | 0.375 | 0.125 |
| chr3: 116452622 | 3 | 116452622 | G/C | 0.4824 | 0.5 | 0.375 | 0.125 |
| chr3: 118148477 | 3 | 118148477 | A/G | 0.5 | 0.5 | 0.375 | 0.125 |
| chr3: 119657526 | 3 | 119657526 | C/T | 0.4864 | 0.5 | 0.375 | 0.125 |
| chr3: 121175246 | 3 | 121175246 | T/C | 0.4984 | 0.5 | 0.375 | 0.125 |
| chr4: 3861427 | 4 | 3861427 | T/C | 0.4988 | 0.5 | 0.375 | 0.125 |
| BovineHD4100002612 | 4 | 5478011 | A/G | 0.4924 | 0.5 | 0.375 | 0.125 |
| BovineHD0400001913 | 4 | 6549577 | A/G | 0.472 | 0.499 | 0.374 | 0.124 |
| BovineHD0400002141 | 4 | 7138504 | T/C | 0.498 | 0.5 | 0.375 | 0.125 |
| chr4: 9021510 | 4 | 9021510 | C/T | 0.4461 | 0.494 | 0.372 | 0.122 |
| chr4: 11577418 | 4 | 11577418 | T/C | 0.4904 | 0.5 | 0.375 | 0.125 |
| chr4: 13098564 | 4 | 13098564 | G/T | 0.4956 | 0.5 | 0.375 | 0.125 |
| chr4: 15359883 | 4 | 15359883 | C/T | 0.3806 | 0.472 | 0.36 | 0.111 |
| chr4: 17779954 | 4 | 17779954 | C/T | 0.4992 | 0.5 | 0.375 | 0.125 |
| chr4: 19078872 | 4 | 19078872 | T/C | 0.4992 | 0.5 | 0.375 | 0.125 |
| chr4: 22645768 | 4 | 22645768 | G/A | 0.4976 | 0.5 | 0.375 | 0.125 |
| chr4: 24307253 | 4 | 24307253 | G/C | 0.403 | 0.481 | 0.365 | 0.116 |
| chr4: 26020550 | 4 | 26020550 | G/C | 0.4992 | 0.5 | 0.375 | 0.125 |
| chr4: 28165801 | 4 | 28165801 | T/G | 0.4269 | 0.49 | 0.37 | 0.12 |
| chr4: 30471896 | 4 | 30471896 | C/A | 0.4944 | 0.5 | 0.375 | 0.125 |
| chr4: 32417299 | 4 | 32417299 | C/A | 0.4984 | 0.5 | 0.375 | 0.125 |
| chr4: 35167308 | 4 | 35167308 | G/T | 0.4996 | 0.5 | 0.375 | 0.125 |
| chr4: 37653081 | 4 | 37653081 | C/T | 0.4996 | 0.5 | 0.375 | 0.125 |
| chr4: 41047723 | 4 | 41047723 | G/A | 0.4784 | 0.499 | 0.375 | 0.125 |
| chr4: 44408279 | 4 | 44408279 | C/A | 0.4956 | 0.5 | 0.375 | 0.125 |
| chr4: 48947495 | 4 | 48947495 | C/A | 0.4996 | 0.5 | 0.375 | 0.125 |
| chr4: 51736700 | 4 | 51736700 | T/C | 0.4984 | 0.5 | 0.375 | 0.125 |
| chr4: 53557315 | 4 | 53557315 | G/A | 0.4517 | 0.496 | 0.373 | 0.123 |
| chr4: 56251739 | 4 | 56251739 | A/T | 0.4617 | 0.497 | 0.374 | 0.124 |
| chr4: 59377877 | 4 | 59377877 | T/A | 0.498 | 0.5 | 0.375 | 0.125 |
| chr4: 62116676 | 4 | 62116676 | T/C | 0.494 | 0.5 | 0.375 | 0.125 |
| chr4: 64220533 | 4 | 64220533 | A/G | 0.4964 | 0.5 | 0.375 | 0.125 |
| chr4: 66072087 | 4 | 66072087 | C/T | 0.4429 | 0.494 | 0.372 | 0.122 |
| chr4: 68072014 | 4 | 68072014 | A/C | 0.4764 | 0.499 | 0.374 | 0.124 |
| chr4: 70958591 | 4 | 70958591 | G/T | 0.4461 | 0.494 | 0.372 | 0.122 |
| chr4: 72419909 | 4 | 72419909 | A/G | 0.4972 | 0.5 | 0.375 | 0.125 |
| chr4: 74319680 | 4 | 74319680 | C/T | 0.4856 | 0.5 | 0.375 | 0.125 |
| chr4: 76069001 | 4 | 76069001 | G/A | 0.4932 | 0.5 | 0.375 | 0.125 |
| chr4: 78733527 | 4 | 78733527 | A/G | 0.4996 | 0.5 | 0.375 | 0.125 |
| chr4: 80412321 | 4 | 80412321 | G/A | 0.4876 | 0.5 | 0.375 | 0.125 |
| chr4: 82284742 | 4 | 82284742 | C/T | 0.4557 | 0.496 | 0.373 | 0.123 |
| chr4: 84205752 | 4 | 84205752 | G/C | 0.4613 | 0.497 | 0.373 | 0.124 |
| chr4: 86290264 | 4 | 86290264 | G/A | 0.4936 | 0.5 | 0.375 | 0.125 |
| chr4: 88179396 | 4 | 88179396 | G/A | 0.4984 | 0.5 | 0.375 | 0.125 |
| chr4: 90103827 | 4 | 90103827 | A/G | 0.4936 | 0.5 | 0.375 | 0.125 |
| chr4: 92469122 | 4 | 92469122 | G/T | 0.4996 | 0.5 | 0.375 | 0.125 |
| chr4: 94734183 | 4 | 94734183 | A/C | 0.4213 | 0.488 | 0.369 | 0.119 |
| chr4: 96100801 | 4 | 96100801 | T/C | 0.4984 | 0.5 | 0.375 | 0.125 |
| chr4: 98539972 | 4 | 98539972 | G/C | 0.4984 | 0.5 | 0.375 | 0.125 |
| chr4: 100397123 | 4 | 100397123 | C/G | 0.4964 | 0.5 | 0.375 | 0.125 |
| chr4: 102560075 | 4 | 102560075 | G/T | 0.4956 | 0.5 | 0.375 | 0.125 |
| chr4: 104245564 | 4 | 104245564 | G/A | 0.4924 | 0.5 | 0.375 | 0.125 |
| chr4: 106099249 | 4 | 106099249 | A/T | 0.494 | 0.5 | 0.375 | 0.125 |
| chr4: 108302747 | 4 | 108302747 | G/C | 0.4996 | 0.5 | 0.375 | 0.125 |
| chr4: 110365140 | 4 | 110365140 | G/A | 0.4988 | 0.5 | 0.375 | 0.125 |
| chr4: 112014359 | 4 | 112014359 | T/C | 0.5 | 0.5 | 0.375 | 0.125 |
| chr4: 114636283 | 4 | 114636283 | C/T | 0.4844 | 0.5 | 0.375 | 0.125 |
| chr4: 116226917 | 4 | 116226917 | C/T | 0.4988 | 0.5 | 0.375 | 0.125 |
| chr4: 119239489 | 4 | 119239489 | T/A | 0.4996 | 0.5 | 0.375 | 0.125 |
| BovineHD0500000007 | 5 | 31485 | C/T | 0.4637 | 0.498 | 0.374 | 0.124 |
| BovineHD0500000460 | 5 | 1797290 | G/T | 0.4992 | 0.5 | 0.375 | 0.125 |
| chr5: 2610987 | 5 | 2610987 | G/T | 0.49 | 0.5 | 0.375 | 0.125 |
| BovineHD0500000773 | 5 | 3012267 | A/G | 0.49 | 0.5 | 0.375 | 0.125 |
| chr5: 5068243 | 5 | 5068243 | G/C | 0.4928 | 0.5 | 0.375 | 0.125 |
| chr5: 6425820 | 5 | 6425820 | T/C | 0.4848 | 0.5 | 0.375 | 0.125 |
| chr5: 8523790 | 5 | 8523790 | A/C | 0.4728 | 0.499 | 0.374 | 0.124 |
| chr5: 9903460 | 5 | 9903460 | A/G | 0.482 | 0.5 | 0.375 | 0.125 |
| chr5: 11064016 | 5 | 11064016 | A/G | 0.4976 | 0.5 | 0.375 | 0.125 |
| chr5: 13468378 | 5 | 13468378 | T/C | 0.4968 | 0.5 | 0.375 | 0.125 |
| chr5: 15650751 | 5 | 15650751 | G/A | 0.5 | 0.5 | 0.375 | 0.125 |
| chr5: 17031046 | 5 | 17031046 | G/T | 0.4868 | 0.5 | 0.375 | 0.125 |
| chr5: 19601695 | 5 | 19601695 | G/A | 0.4665 | 0.498 | 0.374 | 0.124 |
| chr5: 22283765 | 5 | 22283765 | A/C | 0.4952 | 0.5 | 0.375 | 0.125 |
| chr5: 25309909 | 5 | 25309909 | T/G | 0.4884 | 0.5 | 0.375 | 0.125 |
| chr5: 27998047 | 5 | 27998047 | T/G | 0.4613 | 0.497 | 0.373 | 0.124 |
| chr5: 29249825 | 5 | 29249825 | C/T | 0.498 | 0.5 | 0.375 | 0.125 |
| chr5: 32734860 | 5 | 32734860 | G/A | 0.395 | 0.478 | 0.364 | 0.114 |
| chr5: 34934298 | 5 | 34934298 | G/A | 0.4824 | 0.5 | 0.375 | 0.125 |
| chr5: 36100135 | 5 | 36100135 | A/G | 0.4984 | 0.5 | 0.375 | 0.125 |
| chr5: 38860266 | 5 | 38860266 | C/T | 0.4952 | 0.5 | 0.375 | 0.125 |
| chr5: 40539755 | 5 | 40539755 | C/T | 0.4992 | 0.5 | 0.375 | 0.125 |
| chr5: 42546535 | 5 | 42546535 | C/A | 0.5 | 0.5 | 0.375 | 0.125 |
| chr5: 45596322 | 5 | 45596322 | T/C | 0.4756 | 0.499 | 0.374 | 0.124 |
| chr5: 48383398 | 5 | 48383398 | T/A | 0.4984 | 0.5 | 0.375 | 0.125 |
| chr5: 50356341 | 5 | 50356341 | C/T | 0.4992 | 0.5 | 0.375 | 0.125 |
| chr5: 52837788 | 5 | 52837788 | A/G | 0.4984 | 0.5 | 0.375 | 0.125 |
| chr5: 55094769 | 5 | 55094769 | C/A | 0.4996 | 0.5 | 0.375 | 0.125 |
| chr5: 57563283 | 5 | 57563283 | C/T | 0.4992 | 0.5 | 0.375 | 0.125 |
| chr5: 61938027 | 5 | 61938027 | T/C | 0.4988 | 0.5 | 0.375 | 0.125 |
| chr5: 63674646 | 5 | 63674646 | T/C | 0.5 | 0.5 | 0.375 | 0.125 |
| chr5: 65117330 | 5 | 65117330 | T/A | 0.5 | 0.5 | 0.375 | 0.125 |
| chr5: 69355384 | 5 | 69355384 | C/A | 0.498 | 0.5 | 0.375 | 0.125 |
| chr5: 71878536 | 5 | 71878536 | G/A | 0.5 | 0.5 | 0.375 | 0.125 |
| chr5: 74763956 | 5 | 74763956 | C/T | 0.4972 | 0.5 | 0.375 | 0.125 |
| chr5: 75632140 | 5 | 75632140 | C/T | 0.4916 | 0.5 | 0.375 | 0.125 |
| chr5: 78472118 | 5 | 78472118 | C/T | 0.4736 | 0.499 | 0.374 | 0.124 |
| chr5: 80990355 | 5 | 80990355 | G/A | 0.4996 | 0.5 | 0.375 | 0.125 |
| BovineHD0500023615 | 5 | 83434257 | T/C | 0.496 | 0.5 | 0.375 | 0.125 |
| chr5: 85549231 | 5 | 85549231 | G/A | 0.403 | 0.481 | 0.365 | 0.116 |
| chr5: 87397204 | 5 | 87397204 | G/C | 0.4864 | 0.5 | 0.375 | 0.125 |
| chr5: 90018451 | 5 | 90018451 | G/C | 0.4944 | 0.5 | 0.375 | 0.125 |
| chr5: 92365101 | 5 | 92365101 | C/T | 0.4673 | 0.498 | 0.374 | 0.124 |
| chr5: 94219588 | 5 | 94219588 | T/C | 0.4948 | 0.5 | 0.375 | 0.125 |
| chr5: 96207260 | 5 | 96207260 | T/C | 0.4984 | 0.5 | 0.375 | 0.125 |
| chr5: 98156010 | 5 | 98156010 | T/C | 0.4996 | 0.5 | 0.375 | 0.125 |
| chr5: 100451709 | 5 | 100451709 | G/A | 0.5 | 0.5 | 0.375 | 0.125 |
| chr5: 103778185 | 5 | 103778185 | C/A | 0.4952 | 0.5 | 0.375 | 0.125 |
| BovineHD0500030582 | 5 | 106568560 | T/C | 0.472 | 0.499 | 0.374 | 0.124 |
| chr5: 108509149 | 5 | 108509149 | G/A | 0.4621 | 0.497 | 0.374 | 0.124 |
| chr5: 110026273 | 5 | 110026273 | A/G | 0.492 | 0.5 | 0.375 | 0.125 |
| chr5: 112310553 | 5 | 112310553 | C/T | 0.4784 | 0.499 | 0.375 | 0.125 |
| chr5: 114677995 | 5 | 114677995 | A/G | 0.496 | 0.5 | 0.375 | 0.125 |
| chr5: 116585246 | 5 | 116585246 | G/A | 0.4736 | 0.499 | 0.374 | 0.124 |
| chr5: 118068937 | 5 | 118068937 | G/A | 0.4996 | 0.5 | 0.375 | 0.125 |
| chr5: 120597412 | 5 | 120597412 | A/G | 0.4669 | 0.498 | 0.374 | 0.124 |
| BovineHD0600000946 | 6 | 3641139 | A/G | 0.4976 | 0.5 | 0.375 | 0.125 |
| BovineHD0600000994 | 6 | 3807975 | G/A | 0.4996 | 0.5 | 0.375 | 0.125 |
| BovineHD0600001372 | 6 | 5230318 | G/A | 0.3502 | 0.455 | 0.352 | 0.104 |
| BovineHD0600001622 | 6 | 7436488 | A/G | 0.4864 | 0.5 | 0.375 | 0.125 |
| chr6: 8715560 | 6 | 8715560 | T/C | 0.4453 | 0.494 | 0.372 | 0.122 |
| chr6: 10275743 | 6 | 10275743 | T/A | 0.486 | 0.5 | 0.375 | 0.125 |
| chr6: 12025526 | 6 | 12025526 | C/A | 0.4996 | 0.5 | 0.375 | 0.125 |
| chr6: 13851956 | 6 | 13851956 | T/C | 0.4932 | 0.5 | 0.375 | 0.125 |
| chr6: 15923688 | 6 | 15923688 | T/G | 0.4968 | 0.5 | 0.375 | 0.125 |
| chr6: 18191288 | 6 | 18191288 | A/G | 0.4944 | 0.5 | 0.375 | 0.125 |
| chr6: 20029937 | 6 | 20029937 | T/G | 0.49 | 0.5 | 0.375 | 0.125 |
| chr6: 22018522 | 6 | 22018522 | T/G | 0.4964 | 0.5 | 0.375 | 0.125 |
| chr6: 24308611 | 6 | 24308611 | T/C | 0.4389 | 0.493 | 0.371 | 0.121 |
| chr6: 25955274 | 6 | 25955274 | G/A | 0.4868 | 0.5 | 0.375 | 0.125 |
| chr6: 27127135 | 6 | 27127135 | G/A | 0.4984 | 0.5 | 0.375 | 0.125 |
| chr6: 30344055 | 6 | 30344055 | C/T | 0.4868 | 0.5 | 0.375 | 0.125 |
| chr6: 32995850 | 6 | 32995850 | T/C | 0.4988 | 0.5 | 0.375 | 0.125 |
| chr6: 36295718 | 6 | 36295718 | G/T | 0.4776 | 0.499 | 0.374 | 0.125 |
| chr6: 39465845 | 6 | 39465845 | T/C | 0.498 | 0.5 | 0.375 | 0.125 |
| chr6: 42163850 | 6 | 42163850 | G/A | 0.5 | 0.5 | 0.375 | 0.125 |
| chr6: 45430248 | 6 | 45430248 | A/G | 0.4948 | 0.5 | 0.375 | 0.125 |
| chr6: 48405707 | 6 | 48405707 | T/A | 0.4273 | 0.49 | 0.37 | 0.12 |
| chr6: 50368143 | 6 | 50368143 | T/A | 0.4904 | 0.5 | 0.375 | 0.125 |
| chr6: 52091508 | 6 | 52091508 | C/G | 0.4892 | 0.5 | 0.375 | 0.125 |
| chr6: 54763160 | 6 | 54763160 | C/G | 0.4996 | 0.5 | 0.375 | 0.125 |
| chr6: 56962144 | 6 | 56962144 | T/C | 0.4968 | 0.5 | 0.375 | 0.125 |
| chr6: 58716750 | 6 | 58716750 | T/C | 0.4948 | 0.5 | 0.375 | 0.125 |
| chr6: 60131226 | 6 | 60131226 | G/A | 0.496 | 0.5 | 0.375 | 0.125 |
| chr6: 62381859 | 6 | 62381859 | T/C | 0.4996 | 0.5 | 0.375 | 0.125 |
| chr6: 65818645 | 6 | 65818645 | A/G | 0.4996 | 0.5 | 0.375 | 0.125 |
| chr6: 68556443 | 6 | 68556443 | T/C | 0.4984 | 0.5 | 0.375 | 0.125 |
| chr6: 72859610 | 6 | 72859610 | A/G | 0.4972 | 0.5 | 0.375 | 0.125 |
| chr6: 75090494 | 6 | 75090494 | T/C | 0.5 | 0.5 | 0.375 | 0.125 |
| chr6: 87995541 | 6 | 87995541 | G/T | 0.4956 | 0.5 | 0.375 | 0.125 |
| chr6: 97061781 | 6 | 97061781 | G/A | 0.4692 | 0.498 | 0.374 | 0.124 |
| chr6: 108816557 | 6 | 108816557 | C/T | 0.4792 | 0.499 | 0.375 | 0.125 |
| chr6: 117500189 | 6 | 117500189 | T/C | 0.4645 | 0.498 | 0.374 | 0.124 |
| chr7: 323290 | 7 | 323290 | G/A | 0.4437 | 0.494 | 0.372 | 0.122 |
| chr7: 2636459 | 7 | 2636459 | T/C | 0.5 | 0.5 | 0.375 | 0.125 |
| BovineHD0700000816 | 7 | 2920267 | A/G | 0.4992 | 0.5 | 0.375 | 0.125 |
| BovineHD0700000906 | 7 | 3330103 | T/C | 0.4796 | 0.499 | 0.375 | 0.125 |
| chr7: 5497295 | 7 | 5497295 | C/T | 0.4996 | 0.5 | 0.375 | 0.125 |
| chr7: 7969340 | 7 | 7969340 | G/T | 0.4956 | 0.5 | 0.375 | 0.125 |
| chr7: 10003983 | 7 | 10003983 | T/C | 0.4992 | 0.5 | 0.375 | 0.125 |
| chr7: 13202771 | 7 | 13202771 | A/G | 0.4984 | 0.5 | 0.375 | 0.125 |
| chr7: 16922116 | 7 | 16922116 | T/C | 0.4992 | 0.5 | 0.375 | 0.125 |
| chr7: 18729157 | 7 | 18729157 | G/A | 0.4657 | 0.498 | 0.374 | 0.124 |
| chr7: 20314935 | 7 | 20314935 | T/C | 0.4988 | 0.5 | 0.375 | 0.125 |
| chr7: 22363112 | 7 | 22363112 | C/T | 0.4437 | 0.494 | 0.372 | 0.122 |
| chr7: 24361453 | 7 | 24361453 | C/T | 0.4956 | 0.5 | 0.375 | 0.125 |
| BovineHD0700007619 | 7 | 27360142 | G/A | 0.4952 | 0.5 | 0.375 | 0.125 |
| chr7: 29102061 | 7 | 29102061 | A/G | 0.5 | 0.5 | 0.375 | 0.125 |
| chr7: 31395937 | 7 | 31395937 | G/A | 0.4992 | 0.5 | 0.375 | 0.125 |
| chr7: 33488283 | 7 | 33488283 | T/C | 0.4984 | 0.5 | 0.375 | 0.125 |
| chr7: 35127491 | 7 | 35127491 | T/C | 0.4996 | 0.5 | 0.375 | 0.125 |
| chr7: 37079045 | 7 | 37079045 | T/G | 0.4988 | 0.5 | 0.375 | 0.125 |
| BovineHD0700011845 | 7 | 40874649 | A/G | 0.4876 | 0.5 | 0.375 | 0.125 |
| chr7: 42921853 | 7 | 42921853 | T/C | 0.4593 | 0.497 | 0.373 | 0.123 |
| chr7: 46690297 | 7 | 46690297 | G/A | 0.4844 | 0.5 | 0.375 | 0.125 |
| chr7: 48884947 | 7 | 48884947 | A/C | 0.4972 | 0.5 | 0.375 | 0.125 |
| chr7: 50250949 | 7 | 50250949 | G/A | 0.4948 | 0.5 | 0.375 | 0.125 |
| chr7: 53296158 | 7 | 53296158 | G/A | 0.498 | 0.5 | 0.375 | 0.125 |
| chr7: 55798969 | 7 | 55798969 | C/A | 0.4988 | 0.5 | 0.375 | 0.125 |
| chr7: 58187926 | 7 | 58187926 | C/T | 0.4956 | 0.5 | 0.375 | 0.125 |
| chr7: 61088591 | 7 | 61088591 | T/A | 0.4712 | 0.499 | 0.374 | 0.124 |
| chr7: 65393898 | 7 | 65393898 | C/G | 0.494 | 0.5 | 0.375 | 0.125 |
| chr7: 67286820 | 7 | 67286820 | C/G | 0.4844 | 0.5 | 0.375 | 0.125 |
| chr7: 69280676 | 7 | 69280676 | G/A | 0.4972 | 0.5 | 0.375 | 0.125 |
| chr7: 71538319 | 7 | 71538319 | G/A | 0.4972 | 0.5 | 0.375 | 0.125 |
| chr7: 73794744 | 7 | 73794744 | T/C | 0.498 | 0.5 | 0.375 | 0.125 |
| chr7: 75140866 | 7 | 75140866 | G/C | 0.4992 | 0.5 | 0.375 | 0.125 |
| chr7: 77176754 | 7 | 77176754 | C/T | 0.4824 | 0.5 | 0.375 | 0.125 |
| chr7: 79798840 | 7 | 79798840 | C/G | 0.4848 | 0.5 | 0.375 | 0.125 |
| BovineHD0700023890 | 7 | 82015442 | A/C | 0.4988 | 0.5 | 0.375 | 0.125 |
| chr7: 94484358 | 7 | 94484358 | G/A | 0.4888 | 0.5 | 0.375 | 0.125 |
| chr7: 103520087 | 7 | 103520087 | C/T | 0.4964 | 0.5 | 0.375 | 0.125 |
| chr7: 109212612 | 7 | 109212612 | A/C | 0.5 | 0.5 | 0.375 | 0.125 |
| chr8: 52977 | 8 | 52977 | C/T | 0.4996 | 0.5 | 0.375 | 0.125 |
| ARS-USMARC-Parent- | 8 | 1554706 | G/A | 0.4964 | 0.5 | 0.375 | 0.125 |
| DQ916058-rs29016146 | | | | | | | |
| Hapmap46330-BTA-95948 | 8 | 3533444 | T/A | 0.4964 | 0.5 | 0.375 | 0.125 |
| UA-IFASA-5111 | 8 | 3922416 | A/G | 0.4868 | 0.5 | 0.375 | 0.125 |
| chr8: 6024854 | 8 | 6024854 | A/G | 0.4944 | 0.5 | 0.375 | 0.125 |
| chr8: 9054284 | 8 | 9054284 | A/T | 0.3834 | 0.473 | 0.361 | 0.112 |
| chr8: 11592103 | 8 | 11592103 | G/A | 0.4916 | 0.5 | 0.375 | 0.125 |
| chr8: 13246773 | 8 | 13246773 | T/G | 0.4964 | 0.5 | 0.375 | 0.125 |
| chr8: 15507208 | 8 | 15507208 | G/T | 0.4996 | 0.5 | 0.375 | 0.125 |
| chr8: 17122844 | 8 | 17122844 | A/G | 0.4988 | 0.5 | 0.375 | 0.125 |
| chr8: 19494843 | 8 | 19494843 | A/T | 0.4856 | 0.5 | 0.375 | 0.125 |
| chr8: 21255732 | 8 | 21255732 | A/G | 0.4984 | 0.5 | 0.375 | 0.125 |
| chr8: 24452617 | 8 | 24452617 | A/T | 0.4912 | 0.5 | 0.375 | 0.125 |
| BovineHD0800007910 | 8 | 26150623 | A/G | 0.4776 | 0.499 | 0.374 | 0.125 |
| chr8: 29128317 | 8 | 29128317 | T/A | 0.4964 | 0.5 | 0.375 | 0.125 |
| chr8: 31213020 | 8 | 31213020 | C/G | 0.4976 | 0.5 | 0.375 | 0.125 |
| chr8: 32901857 | 8 | 32901857 | A/G | 0.4968 | 0.5 | 0.375 | 0.125 |
| chr8: 34910132 | 8 | 34910132 | T/C | 0.4992 | 0.5 | 0.375 | 0.125 |
| chr8: 37544170 | 8 | 37544170 | G/C | 0.4992 | 0.5 | 0.375 | 0.125 |
| chr8: 39903026 | 8 | 39903026 | T/A | 0.3966 | 0.479 | 0.364 | 0.115 |
| chr8: 41771334 | 8 | 41771334 | A/G | 0.4996 | 0.5 | 0.375 | 0.125 |
| chr8: 42954715 | 8 | 42954715 | C/G | 0.4984 | 0.5 | 0.375 | 0.125 |
| chr8: 45323917 | 8 | 45323917 | A/T | 0.4984 | 0.5 | 0.375 | 0.125 |
| chr8: 47935064 | 8 | 47935064 | A/G | 0.4828 | 0.5 | 0.375 | 0.125 |
| chr8: 50505013 | 8 | 50505013 | A/G | 0.4992 | 0.5 | 0.375 | 0.125 |
| chr8: 52181043 | 8 | 52181043 | A/T | 0.4876 | 0.5 | 0.375 | 0.125 |
| chr8: 54046042 | 8 | 54046042 | C/G | 0.4968 | 0.5 | 0.375 | 0.125 |
| chr8: 56303275 | 8 | 56303275 | C/T | 0.4988 | 0.5 | 0.375 | 0.125 |
| chr8: 58472144 | 8 | 58472144 | T/C | 0.4736 | 0.499 | 0.374 | 0.124 |
| chr8: 60302978 | 8 | 60302978 | C/A | 0.4848 | 0.5 | 0.375 | 0.125 |
| BovineHD0800018780 | 8 | 62878834 | A/G | 0.4996 | 0.5 | 0.375 | 0.125 |
| chr8: 64364896 | 8 | 64364896 | A/G | 0.4924 | 0.5 | 0.375 | 0.125 |
| chr8: 66116974 | 8 | 66116974 | A/G | 0.4916 | 0.5 | 0.375 | 0.125 |
| chr8: 68298246 | 8 | 68298246 | G/A | 0.4864 | 0.5 | 0.375 | 0.125 |
| chr8: 70059755 | 8 | 70059755 | C/T | 0.4824 | 0.5 | 0.375 | 0.125 |
| chr8: 72761382 | 8 | 72761382 | C/A | 0.4988 | 0.5 | 0.375 | 0.125 |
| chr8: 74068851 | 8 | 74068851 | A/G | 0.4984 | 0.5 | 0.375 | 0.125 |
| chr8: 77187323 | 8 | 77187323 | C/T | 0.4916 | 0.5 | 0.375 | 0.125 |
| chr8: 79249577 | 8 | 79249577 | A/T | 0.4952 | 0.5 | 0.375 | 0.125 |
| chr8: 81936011 | 8 | 81936011 | C/T | 0.4976 | 0.5 | 0.375 | 0.125 |
| chr8: 84900873 | 8 | 84900873 | C/T | 0.496 | 0.5 | 0.375 | 0.125 |
| BovineHD0800026578 | 8 | 89625431 | C/T | 0.498 | 0.5 | 0.375 | 0.125 |
| chr8: 91908606 | 8 | 91908606 | G/T | 0.4888 | 0.5 | 0.375 | 0.125 |
| BovineHD0800028888 | 8 | 97833606 | C/T | 0.4952 | 0.5 | 0.375 | 0.125 |
| chr8: 106296663 | 8 | 106296663 | G/A | 0.4996 | 0.5 | 0.375 | 0.125 |
| chr9: 118327 | 9 | 118327 | A/T | 0.486 | 0.5 | 0.375 | 0.125 |
| chr9: 4553903 | 9 | 4553903 | T/C | 0.494 | 0.5 | 0.375 | 0.125 |
| BovineHD0900001546 | 9 | 7173058 | C/A | 0.4992 | 0.5 | 0.375 | 0.125 |
| BovineHD0900002201 | 9 | 9060667 | A/C | 0.4996 | 0.5 | 0.375 | 0.125 |
| BovineHD0900002717 | 9 | 10949242 | A/G | 0.4968 | 0.5 | 0.375 | 0.125 |
| chr9: 11988685 | 9 | 11988685 | A/G | 0.4964 | 0.5 | 0.375 | 0.125 |
| chr9: 14102277 | 9 | 14102277 | C/A | 0.4645 | 0.498 | 0.374 | 0.124 |
| chr9: 16050438 | 9 | 16050438 | T/C | 0.4976 | 0.5 | 0.375 | 0.125 |
| chr9: 18927655 | 9 | 18927655 | T/C | 0.4968 | 0.5 | 0.375 | 0.125 |
| chr9: 20941076 | 9 | 20941076 | T/C | 0.4896 | 0.5 | 0.375 | 0.125 |
| chr9: 23183945 | 9 | 23183945 | T/G | 0.4876 | 0.5 | 0.375 | 0.125 |
| chr9: 25342529 | 9 | 25342529 | G/T | 0.4916 | 0.5 | 0.375 | 0.125 |
| chr9: 27435788 | 9 | 27435788 | T/C | 0.4073 | 0.483 | 0.366 | 0.117 |
| chr9: 29224171 | 9 | 29224171 | T/C | 0.4992 | 0.5 | 0.375 | 0.125 |
| chr9: 31886588 | 9 | 31886588 | G/A | 0.4948 | 0.5 | 0.375 | 0.125 |
| chr9: 33212985 | 9 | 33212985 | T/C | 0.4996 | 0.5 | 0.375 | 0.125 |
| chr9: 35436418 | 9 | 35436418 | C/T | 0.4844 | 0.5 | 0.375 | 0.125 |
| chr9: 37363016 | 9 | 37363016 | G/C | 0.4996 | 0.5 | 0.375 | 0.125 |
| chr9: 39057580 | 9 | 39057580 | G/A | 0.4569 | 0.496 | 0.373 | 0.123 |
| chr9: 41758586 | 9 | 41758586 | A/C | 0.4972 | 0.5 | 0.375 | 0.125 |
| chr9: 43377583 | 9 | 43377583 | C/T | 0.4561 | 0.496 | 0.373 | 0.123 |
| chr9: 44837991 | 9 | 44837991 | A/G | 0.4996 | 0.5 | 0.375 | 0.125 |
| chr9: 47413940 | 9 | 47413940 | C/T | 0.4477 | 0.495 | 0.372 | 0.122 |
| chr9: 49420887 | 9 | 49420887 | C/A | 0.48 | 0.499 | 0.375 | 0.125 |
| chr9: 51267024 | 9 | 51267024 | T/C | 0.4948 | 0.5 | 0.375 | 0.125 |
| chr9: 53379758 | 9 | 53379758 | G/T | 0.4952 | 0.5 | 0.375 | 0.125 |
| chr9: 55422398 | 9 | 55422398 | C/T | 0.4796 | 0.499 | 0.375 | 0.125 |
| chr9: 57258001 | 9 | 57258001 | G/A | 0.4988 | 0.5 | 0.375 | 0.125 |
| chr9: 59236293 | 9 | 59236293 | C/T | 0.4876 | 0.5 | 0.375 | 0.125 |
| chr9: 62650382 | 9 | 62650382 | C/G | 0.4872 | 0.5 | 0.375 | 0.125 |
| chr9: 64191131 | 9 | 64191131 | A/G | 0.478 | 0.499 | 0.375 | 0.125 |
| chr9: 66373455 | 9 | 66373455 | A/C | 0.4952 | 0.5 | 0.375 | 0.125 |
| chr9: 68330426 | 9 | 68330426 | A/C | 0.5 | 0.5 | 0.375 | 0.125 |
| chr9: 70113064 | 9 | 70113064 | C/A | 0.4868 | 0.5 | 0.375 | 0.125 |
| chr9: 72948740 | 9 | 72948740 | A/G | 0.4892 | 0.5 | 0.375 | 0.125 |
| chr9: 74910536 | 9 | 74910536 | C/T | 0.5 | 0.5 | 0.375 | 0.125 |
| chr9: 77198935 | 9 | 77198935 | A/C | 0.4976 | 0.5 | 0.375 | 0.125 |
| chr9: 79682260 | 9 | 79682260 | A/G | 0.4996 | 0.5 | 0.375 | 0.125 |
| chr9: 81526163 | 9 | 81526163 | C/T | 0.4912 | 0.5 | 0.375 | 0.125 |
| chr9: 100656748 | 9 | 100656748 | A/G | 0.4289 | 0.49 | 0.37 | 0.12 |
| chr10: 556085 | 10 | 556085 | T/A | 0.4956 | 0.5 | 0.375 | 0.125 |
| chr10: 1730897 | 10 | 1730897 | A/T | 0.4748 | 0.499 | 0.374 | 0.124 |
| ARS-BFGL-NGS-61586 | 10 | 6413192 | A/G | 0.4956 | 0.5 | 0.375 | 0.125 |
| BovineHD1000002399 | 10 | 7472054 | A/G | 0.4988 | 0.5 | 0.375 | 0.125 |
| chr10: 9153211 | 10 | 9153211 | T/G | 0.4968 | 0.5 | 0.375 | 0.125 |
| BovineHD1000004574 | 10 | 13579099 | C/T | 0.4445 | 0.494 | 0.372 | 0.122 |
| chr10: 15227385 | 10 | 15227385 | C/G | 0.4988 | 0.5 | 0.375 | 0.125 |
| chr10: 17193397 | 10 | 17193397 | G/C | 0.4529 | 0.496 | 0.373 | 0.123 |
| chr10: 19427128 | 10 | 19427128 | C/T | 0.4669 | 0.498 | 0.374 | 0.124 |
| chr10: 21078628 | 10 | 21078628 | C/T | 0.4928 | 0.5 | 0.375 | 0.125 |
| chr10: 23361431 | 10 | 23361431 | C/T | 0.49 | 0.5 | 0.375 | 0.125 |
| chr10: 26456276 | 10 | 26456276 | G/A | 0.4996 | 0.5 | 0.375 | 0.125 |
| chr10: 28994288 | 10 | 28994288 | C/G | 0.4996 | 0.5 | 0.375 | 0.125 |
| chr10: 30582027 | 10 | 30582027 | C/T | 0.4848 | 0.5 | 0.375 | 0.125 |
| chr10: 32552401 | 10 | 32552401 | C/T | 0.3966 | 0.479 | 0.364 | 0.115 |
| chr10: 34431386 | 10 | 34431386 | T/A | 0.4944 | 0.5 | 0.375 | 0.125 |
| BovineHD1000011551 | 10 | 37319439 | A/C | 0.4968 | 0.5 | 0.375 | 0.125 |
| chr10: 39588228 | 10 | 39588228 | A/G | 0.4804 | 0.499 | 0.375 | 0.125 |
| chr10: 41213432 | 10 | 41213432 | G/A | 0.4948 | 0.5 | 0.375 | 0.125 |
| chr10: 43406860 | 10 | 43406860 | A/C | 0.4085 | 0.483 | 0.366 | 0.117 |
| BovineHD1000013921 | 10 | 46503089 | A/G | 0.4976 | 0.5 | 0.375 | 0.125 |
| chr10: 48504197 | 10 | 48504197 | G/C | 0.47 | 0.498 | 0.374 | 0.124 |
| chr10: 50125360 | 10 | 50125360 | G/A | 0.4988 | 0.5 | 0.375 | 0.125 |
| chr10: 52159167 | 10 | 52159167 | T/G | 0.488 | 0.5 | 0.375 | 0.125 |
| chr10: 54236151 | 10 | 54236151 | C/T | 0.4996 | 0.5 | 0.375 | 0.125 |
| chr10: 56198215 | 10 | 56198215 | C/T | 0.4996 | 0.5 | 0.375 | 0.125 |
| chr10: 58765458 | 10 | 58765458 | C/T | 0.4625 | 0.497 | 0.374 | 0.124 |
| chr10: 61728710 | 10 | 61728710 | T/A | 0.4952 | 0.5 | 0.375 | 0.125 |
| chr10: 63028632 | 10 | 63028632 | A/G | 0.4988 | 0.5 | 0.375 | 0.125 |
| chr10: 66983606 | 10 | 66983606 | A/G | 0.4856 | 0.5 | 0.375 | 0.125 |
| chr10: 70944177 | 10 | 70944177 | C/G | 0.4988 | 0.5 | 0.375 | 0.125 |
| chr10: 73413069 | 10 | 73413069 | T/C | 0.4816 | 0.5 | 0.375 | 0.125 |
| chr10: 76359926 | 10 | 76359926 | G/C | 0.4748 | 0.499 | 0.374 | 0.124 |
| chr10: 78335635 | 10 | 78335635 | A/G | 0.4952 | 0.5 | 0.375 | 0.125 |
| chr10: 80305425 | 10 | 80305425 | C/A | 0.4477 | 0.495 | 0.372 | 0.122 |
| chr10: 82391519 | 10 | 82391519 | A/G | 0.4964 | 0.5 | 0.375 | 0.125 |
| chr10: 84072952 | 10 | 84072952 | G/A | 0.4924 | 0.5 | 0.375 | 0.125 |
| chr10: 86098247 | 10 | 86098247 | T/C | 0.4944 | 0.5 | 0.375 | 0.125 |
| chr10: 88459679 | 10 | 88459679 | G/A | 0.3854 | 0.474 | 0.362 | 0.112 |
| chr10: 90765222 | 10 | 90765222 | A/C | 0.492 | 0.5 | 0.375 | 0.125 |
| chr10: 92017959 | 10 | 92017959 | C/T | 0.496 | 0.5 | 0.375 | 0.125 |
| chr10: 94160653 | 10 | 94160653 | C/T | 0.4876 | 0.5 | 0.375 | 0.125 |
| chr10: 96254810 | 10 | 96254810 | G/A | 0.4557 | 0.496 | 0.373 | 0.123 |
| chr10: 98176366 | 10 | 98176366 | C/G | 0.4892 | 0.5 | 0.375 | 0.125 |
| chr10: 102379025 | 10 | 102379025 | G/A | 0.4996 | 0.5 | 0.375 | 0.125 |
| chr11: 1816232 | 11 | 1816232 | C/A | 0.4892 | 0.5 | 0.375 | 0.125 |
| BovineHD1100002103 | 11 | 5778054 | T/C | 0.4864 | 0.5 | 0.375 | 0.125 |
| chr11: 9449037 | 11 | 9449037 | C/T | 0.4984 | 0.5 | 0.375 | 0.125 |
| chr11: 16181304 | 11 | 16181304 | G/A | 0.397 | 0.479 | 0.364 | 0.115 |
| chr11: 18148960 | 11 | 18148960 | T/C | 0.4421 | 0.493 | 0.372 | 0.122 |
| chr11: 20898079 | 11 | 20898079 | G/A | 0.4952 | 0.5 | 0.375 | 0.125 |
| chr11: 23036171 | 11 | 23036171 | C/T | 0.5 | 0.5 | 0.375 | 0.125 |
| BovineHD1100007798 | 11 | 26080039 | C/T | 0.4964 | 0.5 | 0.375 | 0.125 |
| chr11: 28101895 | 11 | 28101895 | G/T | 0.4988 | 0.5 | 0.375 | 0.125 |
| BovineHD1100009590 | 11 | 31928844 | G/A | 0.4948 | 0.5 | 0.375 | 0.125 |
| chr11: 34101385 | 11 | 34101385 | C/T | 0.4677 | 0.498 | 0.374 | 0.124 |
| chr11: 36285809 | 11 | 36285809 | A/G | 0.5 | 0.5 | 0.375 | 0.125 |
| chr11: 39217910 | 11 | 39217910 | C/T | 0.4952 | 0.5 | 0.375 | 0.125 |
| chr11: 42411510 | 11 | 42411510 | T/C | 0.498 | 0.5 | 0.375 | 0.125 |
| chr11: 44850419 | 11 | 44850419 | A/G | 0.4944 | 0.5 | 0.375 | 0.125 |
| BovineHD1100013977 | 11 | 47686080 | G/T | 0.5 | 0.5 | 0.375 | 0.125 |
| chr11: 49991996 | 11 | 49991996 | A/G | 0.4976 | 0.5 | 0.375 | 0.125 |
| chr11: 52270391 | 11 | 52270391 | A/G | 0.4996 | 0.5 | 0.375 | 0.125 |
| chr11: 56101323 | 11 | 56101323 | G/C | 0.4952 | 0.5 | 0.375 | 0.125 |
| chr11: 58578189 | 11 | 58578189 | G/T | 0.4653 | 0.498 | 0.374 | 0.124 |
| chr11: 62962927 | 11 | 62962927 | C/T | 0.4888 | 0.5 | 0.375 | 0.125 |
| chr11: 65495916 | 11 | 65495916 | G/T | 0.4984 | 0.5 | 0.375 | 0.125 |
| chr11: 67558272 | 11 | 67558272 | C/T | 0.4996 | 0.5 | 0.375 | 0.125 |
| chr11: 69888219 | 11 | 69888219 | C/A | 0.4952 | 0.5 | 0.375 | 0.125 |
| chr11: 72119562 | 11 | 72119562 | C/T | 0.4844 | 0.5 | 0.375 | 0.125 |
| chr11: 76815091 | 11 | 76815091 | A/C | 0.4904 | 0.5 | 0.375 | 0.125 |
| chr11: 79430810 | 11 | 79430810 | T/C | 0.4573 | 0.497 | 0.373 | 0.123 |
| BovineHD1100022903 | 11 | 79980970 | T/C | 0.4992 | 0.5 | 0.375 | 0.125 |
| BovineHD1100023475 | 11 | 81959151 | G/T | 0.4976 | 0.5 | 0.375 | 0.125 |
| chr11: 83237290 | 11 | 83237290 | G/A | 0.4808 | 0.499 | 0.375 | 0.125 |
| chr11: 86007382 | 11 | 86007382 | G/C | 0.4856 | 0.5 | 0.375 | 0.125 |
| chr11: 88141810 | 11 | 88141810 | C/T | 0.4728 | 0.499 | 0.374 | 0.124 |
| BovineHD1100027008 | 11 | 92814391 | T/C | 0.5 | 0.5 | 0.375 | 0.125 |
| chr11: 94211519 | 11 | 94211519 | G/T | 0.4988 | 0.5 | 0.375 | 0.125 |
| chr11: 98432605 | 11 | 98432605 | C/T | 0.4964 | 0.5 | 0.375 | 0.125 |
| chr11: 100052622 | 11 | 100052622 | C/A | 0.498 | 0.5 | 0.375 | 0.125 |
| chr11: 103917923 | 11 | 103917923 | G/C | 0.4932 | 0.5 | 0.375 | 0.125 |
| chr12: 1319700 | 12 | 1319700 | A/G | 0.4944 | 0.5 | 0.375 | 0.125 |
| BovineHD1200001590 | 12 | 5423854 | G/A | 0.4884 | 0.5 | 0.375 | 0.125 |
| chr12: 7456083 | 12 | 7456083 | C/T | 0.4968 | 0.5 | 0.375 | 0.125 |
| chr12: 9274719 | 12 | 9274719 | A/G | 0.4916 | 0.5 | 0.375 | 0.125 |
| chr12: 11817572 | 12 | 11817572 | C/A | 0.4996 | 0.5 | 0.375 | 0.125 |
| chr12: 14528558 | 12 | 14528558 | G/C | 0.4928 | 0.5 | 0.375 | 0.125 |
| chr12: 17152561 | 12 | 17152561 | T/C | 0.4852 | 0.5 | 0.375 | 0.125 |
| chr12: 20418884 | 12 | 20418884 | C/T | 0.4988 | 0.5 | 0.375 | 0.125 |
| chr12: 23652977 | 12 | 23652977 | C/T | 0.474 | 0.499 | 0.374 | 0.124 |
| chr12: 24780202 | 12 | 24780202 | T/C | 0.4892 | 0.5 | 0.375 | 0.125 |
| chr12: 26767294 | 12 | 26767294 | T/G | 0.4992 | 0.5 | 0.375 | 0.125 |
| chr12: 29031933 | 12 | 29031933 | C/T | 0.4948 | 0.5 | 0.375 | 0.125 |
| chr12: 31074209 | 12 | 31074209 | C/A | 0.482 | 0.5 | 0.375 | 0.125 |
| chr12: 33937183 | 12 | 33937183 | G/C | 0.4637 | 0.498 | 0.374 | 0.124 |
| chr12: 35685597 | 12 | 35685597 | A/G | 0.4968 | 0.5 | 0.375 | 0.125 |
| chr12: 37689687 | 12 | 37689687 | T/G | 0.4944 | 0.5 | 0.375 | 0.125 |
| chr12: 39546498 | 12 | 39546498 | A/C | 0.4944 | 0.5 | 0.375 | 0.125 |
| chr12: 41364155 | 12 | 41364155 | G/T | 0.4984 | 0.5 | 0.375 | 0.125 |
| chr12: 44447101 | 12 | 44447101 | A/G | 0.4856 | 0.5 | 0.375 | 0.125 |
| chr12: 48802895 | 12 | 48802895 | C/T | 0.4932 | 0.5 | 0.375 | 0.125 |
| chr12: 51164014 | 12 | 51164014 | A/G | 0.4988 | 0.5 | 0.375 | 0.125 |
| chr12: 54348649 | 12 | 54348649 | T/C | 0.492 | 0.5 | 0.375 | 0.125 |
| chr12: 70218372 | 12 | 70218372 | T/C | 0.3926 | 0.477 | 0.363 | 0.114 |
| BovineHD1200022318 | 12 | 78387270 | A/G | 0.496 | 0.5 | 0.375 | 0.125 |
| BTA-30932-no-rs | 12 | 83614359 | G/A | 0.4685 | 0.498 | 0.374 | 0.124 |
| chr12: 87182358 | 12 | 87182358 | C/T | 0.4944 | 0.5 | 0.375 | 0.125 |
| chr13: 1517586 | 13 | 1517586 | C/T | 0.494 | 0.5 | 0.375 | 0.125 |
| BovineHD1300001203 | 13 | 4630391 | A/G | 0.4936 | 0.5 | 0.375 | 0.125 |
| chr13: 7362945 | 13 | 7362945 | C/T | 0.4796 | 0.499 | 0.375 | 0.125 |
| chr13: 9892854 | 13 | 9892854 | G/A | 0.4629 | 0.497 | 0.374 | 0.124 |
| chr13: 13843476 | 13 | 13843476 | A/G | 0.4932 | 0.5 | 0.375 | 0.125 |
| chr13: 15318011 | 13 | 15318011 | T/A | 0.4756 | 0.499 | 0.374 | 0.124 |
| chr13: 17297299 | 13 | 17297299 | G/A | 0.4433 | 0.494 | 0.372 | 0.122 |
| chr13: 19474034 | 13 | 19474034 | C/T | 0.3878 | 0.475 | 0.362 | 0.113 |
| chr13: 21291531 | 13 | 21291531 | C/T | 0.4972 | 0.5 | 0.375 | 0.125 |
| chr13: 23450925 | 13 | 23450925 | T/C | 0.4924 | 0.5 | 0.375 | 0.125 |
| BovineHD4100010034 | 13 | 26047739 | A/C | 0.4964 | 0.5 | 0.375 | 0.125 |
| chr13: 28918149 | 13 | 28918149 | C/G | 0.4046 | 0.482 | 0.366 | 0.116 |
| chr13: 30005237 | 13 | 30005237 | C/T | 0.4964 | 0.5 | 0.375 | 0.125 |
| chr13: 32728171 | 13 | 32728171 | T/C | 0.4808 | 0.499 | 0.375 | 0.125 |
| chr13: 36138561 | 13 | 36138561 | G/A | 0.4964 | 0.5 | 0.375 | 0.125 |
| chr13: 39437219 | 13 | 39437219 | G/A | 0.4996 | 0.5 | 0.375 | 0.125 |
| chr13: 41477551 | 13 | 41477551 | A/G | 0.4928 | 0.5 | 0.375 | 0.125 |
| chr13: 44551336 | 13 | 44551336 | C/G | 0.494 | 0.5 | 0.375 | 0.125 |
| BovineHD1300013764 | 13 | 46950685 | C/T | 0.4557 | 0.496 | 0.373 | 0.123 |
| BTB-01125985 | 13 | 51118474 | C/A | 0.496 | 0.5 | 0.375 | 0.125 |
| chr13: 58768742 | 13 | 58768742 | G/A | 0.492 | 0.5 | 0.375 | 0.125 |
| BovineHD1300018739 | 13 | 65998236 | A/G | 0.4868 | 0.5 | 0.375 | 0.125 |
| chr13: 70658985 | 13 | 70658985 | T/C | 0.4912 | 0.5 | 0.375 | 0.125 |
| BovineHD1300024312 | 13 | 83560269 | A/G | 0.4984 | 0.5 | 0.375 | 0.125 |
| chr14: 358943 | 14 | 358943 | T/C | 0.4605 | 0.497 | 0.373 | 0.123 |
| chr14: 3080193 | 14 | 3080193 | G/A | 0.4928 | 0.5 | 0.375 | 0.125 |
| chr14: 6491593 | 14 | 6491593 | T/C | 0.4545 | 0.496 | 0.373 | 0.123 |
| Hapmap54453-rs29015999 | 14 | 10812434 | C/T | 0.4976 | 0.5 | 0.375 | 0.125 |
| chr14: 12207325 | 14 | 12207325 | C/T | 0.4968 | 0.5 | 0.375 | 0.125 |
| BovineHD1400004049 | 14 | 14029735 | T/C | 0.4876 | 0.5 | 0.375 | 0.125 |
| BovineHD1400005023 | 14 | 17624295 | G/A | 0.496 | 0.5 | 0.375 | 0.125 |
| chr14: 19580120 | 14 | 19580120 | T/C | 0.4992 | 0.5 | 0.375 | 0.125 |
| chr14: 21343979 | 14 | 21343979 | A/G | 0.498 | 0.5 | 0.375 | 0.125 |
| chr14: 23436581 | 14 | 23436581 | G/A | 0.498 | 0.5 | 0.375 | 0.125 |
| chr14: 25897491 | 14 | 25897491 | G/A | 0.5 | 0.5 | 0.375 | 0.125 |
| chr14: 27996180 | 14 | 27996180 | C/A | 0.4972 | 0.5 | 0.375 | 0.125 |
| chr14: 29576356 | 14 | 29576356 | T/A | 0.4972 | 0.5 | 0.375 | 0.125 |
| chr14: 31898098 | 14 | 31898098 | T/C | 0.4988 | 0.5 | 0.375 | 0.125 |
| BovineHD1400010360 | 14 | 35973935 | T/C | 0.4573 | 0.497 | 0.373 | 0.123 |
| chr14: 38491622 | 14 | 38491622 | A/G | 0.4992 | 0.5 | 0.375 | 0.125 |
| chr14: 40835506 | 14 | 40835506 | G/A | 0.4828 | 0.5 | 0.375 | 0.125 |
| chr14: 42946639 | 14 | 42946639 | C/G | 0.3806 | 0.472 | 0.36 | 0.111 |
| chr14: 44071226 | 14 | 44071226 | A/C | 0.4976 | 0.5 | 0.375 | 0.125 |
| chr14: 47444785 | 14 | 47444785 | A/G | 0.4996 | 0.5 | 0.375 | 0.125 |
| chr14: 49311562 | 14 | 49311562 | T/C | 0.4984 | 0.5 | 0.375 | 0.125 |
| chr14: 51783709 | 14 | 51783709 | A/G | 0.4824 | 0.5 | 0.375 | 0.125 |
| BovineHD1400014971 | 14 | 53111814 | C/T | 0.488 | 0.5 | 0.375 | 0.125 |
| BovineHD4100011609 | 14 | 56726986 | C/T | 0.4724 | 0.499 | 0.374 | 0.124 |
| chr14: 58774396 | 14 | 58774396 | A/T | 0.4976 | 0.5 | 0.375 | 0.125 |
| chr14: 61264006 | 14 | 61264006 | C/T | 0.4896 | 0.5 | 0.375 | 0.125 |
| chr14: 63211114 | 14 | 63211114 | A/T | 0.4034 | 0.482 | 0.365 | 0.116 |
| chr14: 65283781 | 14 | 65283781 | G/A | 0.4964 | 0.5 | 0.375 | 0.125 |
| chr14: 68940797 | 14 | 68940797 | C/T | 0.5 | 0.5 | 0.375 | 0.125 |
| chr14: 73268228 | 14 | 73268228 | G/A | 0.4984 | 0.5 | 0.375 | 0.125 |
| BovineHD1400023889 | 14 | 84283231 | T/C | 0.4992 | 0.5 | 0.375 | 0.125 |
| chr15: 705104 | 15 | 705104 | A/G | 0.4996 | 0.5 | 0.375 | 0.125 |
| chr15: 1426673 | 15 | 1426673 | A/G | 0.486 | 0.5 | 0.375 | 0.125 |
| chr15: 13966063 | 15 | 13966063 | A/T | 0.4968 | 0.5 | 0.375 | 0.125 |
| chr15: 15353085 | 15 | 15353085 | T/C | 0.4976 | 0.5 | 0.375 | 0.125 |
| chr15: 17863316 | 15 | 17863316 | A/G | 0.5 | 0.5 | 0.375 | 0.125 |
| chr15: 20577613 | 15 | 20577613 | T/G | 0.4984 | 0.5 | 0.375 | 0.125 |
| chr15: 22753967 | 15 | 22753967 | G/A | 0.4996 | 0.5 | 0.375 | 0.125 |
| chr15: 24704857 | 15 | 24704857 | A/C | 0.4896 | 0.5 | 0.375 | 0.125 |
| chr15: 26010177 | 15 | 26010177 | C/T | 0.4876 | 0.5 | 0.375 | 0.125 |
| chr15: 28586304 | 15 | 28586304 | C/G | 0.4685 | 0.498 | 0.374 | 0.124 |
| chr15: 31931965 | 15 | 31931965 | T/C | 0.4996 | 0.5 | 0.375 | 0.125 |
| chr15: 33279400 | 15 | 33279400 | T/A | 0.4561 | 0.496 | 0.373 | 0.123 |
| chr15: 34120648 | 15 | 34120648 | T/G | 0.488 | 0.5 | 0.375 | 0.125 |
| chr15: 35353463 | 15 | 35353463 | C/A | 0.4988 | 0.5 | 0.375 | 0.125 |
| chr15: 36631768 | 15 | 36631768 | A/G | 0.4996 | 0.5 | 0.375 | 0.125 |
| chr15: 37936745 | 15 | 37936745 | T/C | 0.4988 | 0.5 | 0.375 | 0.125 |
| chr15: 41112170 | 15 | 41112170 | G/A | 0.4852 | 0.5 | 0.375 | 0.125 |
| chr15: 43478045 | 15 | 43478045 | T/C | 0.4896 | 0.5 | 0.375 | 0.125 |
| chr15: 45153486 | 15 | 45153486 | C/A | 0.4952 | 0.5 | 0.375 | 0.125 |
| chr15: 47802027 | 15 | 47802027 | C/T | 0.4856 | 0.5 | 0.375 | 0.125 |
| chr15: 49090962 | 15 | 49090962 | G/A | 0.4972 | 0.5 | 0.375 | 0.125 |
| chr15: 51607362 | 15 | 51607362 | T/C | 0.4972 | 0.5 | 0.375 | 0.125 |
| chr15: 53006024 | 15 | 53006024 | T/C | 0.4936 | 0.5 | 0.375 | 0.125 |
| BovineHD1500016013 | 15 | 55392541 | G/A | 0.4996 | 0.5 | 0.375 | 0.125 |
| chr15: 58357732 | 15 | 58357732 | A/G | 0.4964 | 0.5 | 0.375 | 0.125 |
| chr15: 61616402 | 15 | 61616402 | C/T | 0.4964 | 0.5 | 0.375 | 0.125 |
| chr15: 64303035 | 15 | 64303035 | A/C | 0.4505 | 0.495 | 0.373 | 0.123 |
| chr15: 67002416 | 15 | 67002416 | T/C | 0.4597 | 0.497 | 0.373 | 0.123 |
| chr15: 70085418 | 15 | 70085418 | G/A | 0.4984 | 0.5 | 0.375 | 0.125 |
| chr15: 72322212 | 15 | 72322212 | C/T | 0.4561 | 0.496 | 0.373 | 0.123 |
| chr15: 75697490 | 15 | 75697490 | A/G | 0.4984 | 0.5 | 0.375 | 0.125 |
| chr15: 79573827 | 15 | 79573827 | T/A | 0.4908 | 0.5 | 0.375 | 0.125 |
| chr15: 82767382 | 15 | 82767382 | C/T | 0.4976 | 0.5 | 0.375 | 0.125 |
| chr15: 84713024 | 15 | 84713024 | G/A | 0.49 | 0.5 | 0.375 | 0.125 |
| chr16: 77616 | 16 | 77616 | A/C | 0.4852 | 0.5 | 0.375 | 0.125 |
| ARS-BFGL-NGS-10856 | 16 | 1690484 | C/T | 0.4896 | 0.5 | 0.375 | 0.125 |
| chr16: 3834931 | 16 | 3834931 | A/G | 0.4828 | 0.5 | 0.375 | 0.125 |
| chr16: 5283226 | 16 | 5283226 | G/A | 0.4916 | 0.5 | 0.375 | 0.125 |
| chr16: 7392772 | 16 | 7392772 | C/G | 0.4968 | 0.5 | 0.375 | 0.125 |
| chr16: 9445946 | 16 | 9445946 | T/C | 0.4984 | 0.5 | 0.375 | 0.125 |
| chr16: 11735482 | 16 | 11735482 | G/C | 0.4976 | 0.5 | 0.375 | 0.125 |
| chr16: 12752913 | 16 | 12752913 | C/T | 0.4824 | 0.5 | 0.375 | 0.125 |
| chr16: 14240346 | 16 | 14240346 | C/T | 0.4936 | 0.5 | 0.375 | 0.125 |
| chr16: 15357072 | 16 | 15357072 | A/G | 0.4353 | 0.492 | 0.371 | 0.121 |
| chr16: 18018781 | 16 | 18018781 | T/C | 0.4241 | 0.489 | 0.369 | 0.119 |
| chr16: 19501245 | 16 | 19501245 | C/T | 0.4968 | 0.5 | 0.375 | 0.125 |
| chr16: 21269494 | 16 | 21269494 | G/A | 0.4221 | 0.488 | 0.369 | 0.119 |
| chr16: 23486555 | 16 | 23486555 | G/T | 0.4976 | 0.5 | 0.375 | 0.125 |
| BovineHD1600008020 | 16 | 28632394 | C/T | 0.4996 | 0.5 | 0.375 | 0.125 |
| chr16: 34333202 | 16 | 34333202 | A/G | 0.4844 | 0.5 | 0.375 | 0.125 |
| chr16: 36817430 | 16 | 36817430 | A/T | 0.4968 | 0.5 | 0.375 | 0.125 |
| chr16: 38789947 | 16 | 38789947 | A/G | 0.4996 | 0.5 | 0.375 | 0.125 |
| chr16: 41098125 | 16 | 41098125 | A/G | 0.498 | 0.5 | 0.375 | 0.125 |
| chr16: 44671987 | 16 | 44671987 | G/A | 0.4201 | 0.487 | 0.369 | 0.119 |
| chr16: 46410126 | 16 | 46410126 | C/T | 0.498 | 0.5 | 0.375 | 0.125 |
| chr16: 48803795 | 16 | 48803795 | G/A | 0.496 | 0.5 | 0.375 | 0.125 |
| chr16: 50602909 | 16 | 50602909 | A/G | 0.5 | 0.5 | 0.375 | 0.125 |
| BovineHD1600014931 | 16 | 53827496 | C/T | 0.3686 | 0.466 | 0.357 | 0.108 |
| chr16: 55295999 | 16 | 55295999 | G/A | 0.4964 | 0.5 | 0.375 | 0.125 |
| chr16: 57769675 | 16 | 57769675 | A/G | 0.4952 | 0.5 | 0.375 | 0.125 |
| chr16: 59139262 | 16 | 59139262 | G/T | 0.4896 | 0.5 | 0.375 | 0.125 |
| chr16: 61094760 | 16 | 61094760 | C/A | 0.4904 | 0.5 | 0.375 | 0.125 |
| chr16: 63552899 | 16 | 63552899 | G/A | 0.4984 | 0.5 | 0.375 | 0.125 |
| chr16: 65136800 | 16 | 65136800 | A/G | 0.4964 | 0.5 | 0.375 | 0.125 |
| chr16: 67144941 | 16 | 67144941 | C/T | 0.4984 | 0.5 | 0.375 | 0.125 |
| chr16: 69668766 | 16 | 69668766 | T/C | 0.4952 | 0.5 | 0.375 | 0.125 |
| chr16: 71320729 | 16 | 71320729 | G/A | 0.4661 | 0.498 | 0.374 | 0.124 |
| chr16: 73804367 | 16 | 73804367 | G/T | 0.492 | 0.5 | 0.375 | 0.125 |
| chr16: 75591725 | 16 | 75591725 | G/C | 0.4932 | 0.5 | 0.375 | 0.125 |
| chr16: 78837702 | 16 | 78837702 | G/A | 0.472 | 0.499 | 0.374 | 0.124 |
| chr16: 80439174 | 16 | 80439174 | C/T | 0.4868 | 0.5 | 0.375 | 0.125 |
| chr16: 81680738 | 16 | 81680738 | G/A | 0.3534 | 0.457 | 0.353 | 0.104 |
| chr17: 3949 | 17 | 3949 | A/G | 0.365 | 0.464 | 0.356 | 0.107 |
| BovineHD1700000599 | 17 | 2697332 | C/T | 0.4972 | 0.5 | 0.375 | 0.125 |
| chr17: 4148744 | 17 | 4148744 | G/C | 0.4992 | 0.5 | 0.375 | 0.125 |
| chr17: 6329595 | 17 | 6329595 | C/T | 0.4788 | 0.499 | 0.375 | 0.125 |
| chr17: 9313408 | 17 | 9313408 | C/T | 0.4904 | 0.5 | 0.375 | 0.125 |
| chr17: 13875510 | 17 | 13875510 | T/C | 0.4996 | 0.5 | 0.375 | 0.125 |
| chr17: 15091533 | 17 | 15091533 | A/G | 0.494 | 0.5 | 0.375 | 0.125 |
| chr17: 17047819 | 17 | 17047819 | G/A | 0.4253 | 0.489 | 0.369 | 0.119 |
| chr17: 19422833 | 17 | 19422833 | T/C | 0.4996 | 0.5 | 0.375 | 0.125 |
| chr17: 21001768 | 17 | 21001768 | C/G | 0.4996 | 0.5 | 0.375 | 0.125 |
| chr17: 23725618 | 17 | 23725618 | A/G | 0.4984 | 0.5 | 0.375 | 0.125 |
| chr17: 25823983 | 17 | 25823983 | T/C | 0.4992 | 0.5 | 0.375 | 0.125 |
| chr17: 28268900 | 17 | 28268900 | C/T | 0.4996 | 0.5 | 0.375 | 0.125 |
| chr17: 32282994 | 17 | 32282994 | G/C | 0.4904 | 0.5 | 0.375 | 0.125 |
| chr17: 34678792 | 17 | 34678792 | C/T | 0.4964 | 0.5 | 0.375 | 0.125 |
| chr17: 37147703 | 17 | 37147703 | T/A | 0.4888 | 0.5 | 0.375 | 0.125 |
| chr17: 39057027 | 17 | 39057027 | A/G | 0.488 | 0.5 | 0.375 | 0.125 |
| BovineHD1700013264 | 17 | 47362503 | A/G | 0.4996 | 0.5 | 0.375 | 0.125 |
| chr17: 50542329 | 17 | 50542329 | T/C | 0.4992 | 0.5 | 0.375 | 0.125 |
| chr17: 52756216 | 17 | 52756216 | G/A | 0.5 | 0.5 | 0.375 | 0.125 |
| chr17: 57107543 | 17 | 57107543 | G/T | 0.4832 | 0.5 | 0.375 | 0.125 |
| chr17: 60037087 | 17 | 60037087 | C/A | 0.4996 | 0.5 | 0.375 | 0.125 |
| chr17: 62141105 | 17 | 62141105 | T/A | 0.4964 | 0.5 | 0.375 | 0.125 |
| chr17: 72829287 | 17 | 72829287 | T/C | 0.4716 | 0.499 | 0.374 | 0.124 |
| chr18: 1103982 | 18 | 1103982 | G/A | 0.4936 | 0.5 | 0.375 | 0.125 |
| BovineHD1800000964 | 18 | 3336203 | G/A | 0.4988 | 0.5 | 0.375 | 0.125 |
| BovineHD1800001350 | 18 | 4617525 | A/C | 0.4992 | 0.5 | 0.375 | 0.125 |
| chr18: 5353628 | 18 | 5353628 | C/T | 0.4836 | 0.5 | 0.375 | 0.125 |
| chr18: 7950372 | 18 | 7950372 | A/G | 0.4984 | 0.5 | 0.375 | 0.125 |
| chr18: 10756875 | 18 | 10756875 | G/A | 0.4984 | 0.5 | 0.375 | 0.125 |
| chr18: 12449822 | 18 | 12449822 | A/C | 0.4912 | 0.5 | 0.375 | 0.125 |
| chr18: 14710828 | 18 | 14710828 | C/A | 0.4601 | 0.497 | 0.373 | 0.123 |
| chr18: 16187740 | 18 | 16187740 | G/A | 0.494 | 0.5 | 0.375 | 0.125 |
| chr18: 18435022 | 18 | 18435022 | G/A | 0.4968 | 0.5 | 0.375 | 0.125 |
| chr18: 20350656 | 18 | 20350656 | A/G | 0.4984 | 0.5 | 0.375 | 0.125 |
| BovineHD1800006908 | 18 | 22442470 | C/T | 0.4984 | 0.5 | 0.375 | 0.125 |
| chr18: 24273529 | 18 | 24273529 | A/G | 0.5 | 0.5 | 0.375 | 0.125 |
| chr18: 26690572 | 18 | 26690572 | A/C | 0.4724 | 0.499 | 0.374 | 0.124 |
| chr18: 29189137 | 18 | 29189137 | T/C | 0.4972 | 0.5 | 0.375 | 0.125 |
| chr18: 30525057 | 18 | 30525057 | G/A | 0.49 | 0.5 | 0.375 | 0.125 |
| chr18: 32419138 | 18 | 32419138 | A/G | 0.4996 | 0.5 | 0.375 | 0.125 |
| chr18: 35199090 | 18 | 35199090 | A/G | 0.5 | 0.5 | 0.375 | 0.125 |
| chr18: 36326398 | 18 | 36326398 | C/A | 0.4832 | 0.5 | 0.375 | 0.125 |
| chr18: 39871022 | 18 | 39871022 | A/G | 0.4968 | 0.5 | 0.375 | 0.125 |
| BovineHD1800012278 | 18 | 41585347 | G/A | 0.4545 | 0.496 | 0.373 | 0.123 |
| chr18: 44684830 | 18 | 44684830 | C/T | 0.4804 | 0.499 | 0.375 | 0.125 |
| chr18: 47852446 | 18 | 47852446 | A/G | 0.4944 | 0.5 | 0.375 | 0.125 |
| chr18: 50636269 | 18 | 50636269 | C/T | 0.4605 | 0.497 | 0.373 | 0.123 |
| BovineHD1900000881 | 19 | 3780300 | G/A | 0.5 | 0.5 | 0.375 | 0.125 |
| BovineHD1900001085 | 19 | 4588941 | A/G | 0.4996 | 0.5 | 0.375 | 0.125 |
| BovineHD1900002258 | 19 | 8324786 | T/C | 0.4984 | 0.5 | 0.375 | 0.125 |
| chr19: 9589798 | 19 | 9589798 | G/T | 0.4836 | 0.5 | 0.375 | 0.125 |
| chr19: 11774713 | 19 | 11774713 | T/C | 0.4972 | 0.5 | 0.375 | 0.125 |
| chr19: 13478080 | 19 | 13478080 | G/C | 0.4952 | 0.5 | 0.375 | 0.125 |
| BovineHD1900003935 | 19 | 14774749 | G/A | 0.4633 | 0.497 | 0.374 | 0.124 |
| chr19: 15369234 | 19 | 15369234 | A/G | 0.4712 | 0.499 | 0.374 | 0.124 |
| chr19: 18798193 | 19 | 18798193 | T/A | 0.4992 | 0.5 | 0.375 | 0.125 |
| chr19: 20993189 | 19 | 20993189 | C/T | 0.4972 | 0.5 | 0.375 | 0.125 |
| chr19: 22593676 | 19 | 22593676 | A/T | 0.4944 | 0.5 | 0.375 | 0.125 |
| chr19: 24034883 | 19 | 24034883 | T/G | 0.4661 | 0.498 | 0.374 | 0.124 |
| chr19: 28007039 | 19 | 28007039 | T/G | 0.4988 | 0.5 | 0.375 | 0.125 |
| chr19: 30734931 | 19 | 30734931 | T/G | 0.4329 | 0.491 | 0.37 | 0.121 |
| chr19: 33298629 | 19 | 33298629 | G/C | 0.4944 | 0.5 | 0.375 | 0.125 |
| chr19: 36967680 | 19 | 36967680 | A/G | 0.4968 | 0.5 | 0.375 | 0.125 |
| chr19: 38804144 | 19 | 38804144 | G/C | 0.492 | 0.5 | 0.375 | 0.125 |
| chr19: 40065445 | 19 | 40065445 | G/A | 0.4984 | 0.5 | 0.375 | 0.125 |
| chr19: 42493450 | 19 | 42493450 | C/T | 0.4976 | 0.5 | 0.375 | 0.125 |
| chr19: 44405075 | 19 | 44405075 | A/G | 0.4732 | 0.499 | 0.374 | 0.124 |
| chr19: 46011586 | 19 | 46011586 | G/C | 0.4816 | 0.5 | 0.375 | 0.125 |
| chr19: 48332465 | 19 | 48332465 | G/A | 0.4708 | 0.498 | 0.374 | 0.124 |
| chr19: 50508663 | 19 | 50508663 | T/C | 0.4768 | 0.499 | 0.374 | 0.124 |
| BovineHD1900015032 | 19 | 53717829 | C/T | 0.4968 | 0.5 | 0.375 | 0.125 |
| chr19: 55308139 | 19 | 55308139 | A/C | 0.4976 | 0.5 | 0.375 | 0.125 |
| chr19: 57917130 | 19 | 57917130 | G/T | 0.4976 | 0.5 | 0.375 | 0.125 |
| chr19: 59940672 | 19 | 59940672 | G/T | 0.486 | 0.5 | 0.375 | 0.125 |
| chr19: 62032271 | 19 | 62032271 | A/G | 0.4944 | 0.5 | 0.375 | 0.125 |
| chr19: 64016415 | 19 | 64016415 | A/G | 0.4525 | 0.496 | 0.373 | 0.123 |
| BovineHD2000000176 | 20 | 618962 | C/A | 0.5 | 0.5 | 0.375 | 0.125 |
| chr20: 1453145 | 20 | 1453145 | G/C | 0.4996 | 0.5 | 0.375 | 0.125 |
| chr20: 3233514 | 20 | 3233514 | A/C | 0.3798 | 0.471 | 0.36 | 0.111 |
| BovineHD2000001651 | 20 | 5217883 | A/G | 0.5 | 0.5 | 0.375 | 0.125 |
| Hapmap39533-BTA-108830 | 20 | 7143287 | A/C | 0.4964 | 0.5 | 0.375 | 0.125 |
| chr20: 9325003 | 20 | 9325003 | T/C | 0.4673 | 0.498 | 0.374 | 0.124 |
| chr20: 11247384 | 20 | 11247384 | A/T | 0.4984 | 0.5 | 0.375 | 0.125 |
| chr20: 13170776 | 20 | 13170776 | T/C | 0.4832 | 0.5 | 0.375 | 0.125 |
| chr20: 14423688 | 20 | 14423688 | A/G | 0.4405 | 0.493 | 0.371 | 0.121 |
| chr20: 16164218 | 20 | 16164218 | G/T | 0.4872 | 0.5 | 0.375 | 0.125 |
| chr20: 18196116 | 20 | 18196116 | G/A | 0.4996 | 0.5 | 0.375 | 0.125 |
| chr20: 20454853 | 20 | 20454853 | A/G | 0.4984 | 0.5 | 0.375 | 0.125 |
| chr20: 22542502 | 20 | 22542502 | C/T | 0.4992 | 0.5 | 0.375 | 0.125 |
| chr20: 24625191 | 20 | 24625191 | G/A | 0.4992 | 0.5 | 0.375 | 0.125 |
| chr20: 26041808 | 20 | 26041808 | G/A | 0.4832 | 0.5 | 0.375 | 0.125 |
| chr20: 28033599 | 20 | 28033599 | A/G | 0.4912 | 0.5 | 0.375 | 0.125 |
| chr20: 30619168 | 20 | 30619168 | G/A | 0.4964 | 0.5 | 0.375 | 0.125 |
| chr20: 32945313 | 20 | 32945313 | T/G | 0.4992 | 0.5 | 0.375 | 0.125 |
| BovineHD2000010338 | 20 | 36100687 | A/C | 0.4944 | 0.5 | 0.375 | 0.125 |
| chr20: 38864330 | 20 | 38864330 | G/A | 0.4836 | 0.5 | 0.375 | 0.125 |
| chr20: 40184905 | 20 | 40184905 | G/A | 0.4988 | 0.5 | 0.375 | 0.125 |
| chr20: 44132733 | 20 | 44132733 | C/A | 0.4872 | 0.5 | 0.375 | 0.125 |
| BovineHD2000016809 | 20 | 59809528 | C/T | 0.4653 | 0.498 | 0.374 | 0.124 |
| BovineHD2000019528 | 20 | 67358359 | A/G | 0.4996 | 0.5 | 0.375 | 0.125 |
| chr21: 672490 | 21 | 672490 | T/C | 0.3714 | 0.467 | 0.358 | 0.109 |
| BovineHD2100000817 | 21 | 4753621 | T/C | 0.4964 | 0.5 | 0.375 | 0.125 |
| BovineHD2100001634 | 21 | 7558076 | C/T | 0.4988 | 0.5 | 0.375 | 0.125 |
| ARS-BFGL-NGS-58825 | 21 | 12917017 | G/T | 0.4984 | 0.5 | 0.375 | 0.125 |
| BovineHD2100003468 | 21 | 13235956 | A/G | 0.488 | 0.5 | 0.375 | 0.125 |
| chr21: 16243319 | 21 | 16243319 | T/C | 0.4792 | 0.499 | 0.375 | 0.125 |
| chr21: 18091875 | 21 | 18091875 | A/G | 0.4964 | 0.5 | 0.375 | 0.125 |
| chr21: 19663762 | 21 | 19663762 | C/T | 0.4928 | 0.5 | 0.375 | 0.125 |
| chr21: 21677147 | 21 | 21677147 | C/G | 0.4932 | 0.5 | 0.375 | 0.125 |
| chr21: 23145931 | 21 | 23145931 | T/C | 0.4968 | 0.5 | 0.375 | 0.125 |
| chr21: 25634112 | 21 | 25634112 | G/A | 0.4844 | 0.5 | 0.375 | 0.125 |
| chr21: 27140935 | 21 | 27140935 | T/C | 0.4932 | 0.5 | 0.375 | 0.125 |
| chr21: 28854577 | 21 | 28854577 | A/T | 0.4952 | 0.5 | 0.375 | 0.125 |
| chr21: 31440243 | 21 | 31440243 | G/A | 0.4996 | 0.5 | 0.375 | 0.125 |
| chr21: 33089284 | 21 | 33089284 | G/A | 0.4445 | 0.494 | 0.372 | 0.122 |
| BovineHD2100010614 | 21 | 36212484 | G/T | 0.5 | 0.5 | 0.375 | 0.125 |
| chr21: 38895994 | 21 | 38895994 | T/C | 0.498 | 0.5 | 0.375 | 0.125 |
| chr21: 42412964 | 21 | 42412964 | T/C | 0.47 | 0.498 | 0.374 | 0.124 |
| chr21: 44091783 | 21 | 44091783 | T/A | 0.5 | 0.5 | 0.375 | 0.125 |
| chr21: 46136490 | 21 | 46136490 | C/T | 0.4996 | 0.5 | 0.375 | 0.125 |
| chr21: 49990739 | 21 | 49990739 | G/A | 0.4996 | 0.5 | 0.375 | 0.125 |
| chr21: 52183591 | 21 | 52183591 | T/A | 0.498 | 0.5 | 0.375 | 0.125 |
| chr21: 54577771 | 21 | 54577771 | G/C | 0.4293 | 0.49 | 0.37 | 0.12 |
| chr21: 56258756 | 21 | 56258756 | T/G | 0.4301 | 0.49 | 0.37 | 0.12 |
| chr21: 59331618 | 21 | 59331618 | T/G | 0.4792 | 0.499 | 0.375 | 0.125 |
| chr21: 68760593 | 21 | 68760593 | C/T | 0.4413 | 0.493 | 0.372 | 0.122 |
| BovineHD2200001407 | 22 | 5059795 | C/T | 0.496 | 0.5 | 0.375 | 0.125 |
| BovineHD2200002001 | 22 | 6637551 | T/C | 0.4305 | 0.491 | 0.37 | 0.12 |
| chr22: 6781996 | 22 | 6781996 | G/C | 0.4916 | 0.5 | 0.375 | 0.125 |
| chr22: 9048837 | 22 | 9048837 | C/T | 0.49 | 0.5 | 0.375 | 0.125 |
| chr22: 11789736 | 22 | 11789736 | A/G | 0.4457 | 0.494 | 0.372 | 0.122 |
| chr22: 14617876 | 22 | 14617876 | C/T | 0.496 | 0.5 | 0.375 | 0.125 |
| chr22: 16936005 | 22 | 16936005 | T/C | 0.4932 | 0.5 | 0.375 | 0.125 |
| ARS-BFGL-NGS-35294 | 22 | 18774368 | G/A | 0.4928 | 0.5 | 0.375 | 0.125 |
| chr22: 20588609 | 22 | 20588609 | T/C | 0.4633 | 0.497 | 0.374 | 0.124 |
| chr22: 22391024 | 22 | 22391024 | A/G | 0.5 | 0.5 | 0.375 | 0.125 |
| chr22: 24938859 | 22 | 24938859 | C/A | 0.4956 | 0.5 | 0.375 | 0.125 |
| chr22: 27440977 | 22 | 27440977 | G/A | 0.4952 | 0.5 | 0.375 | 0.125 |
| chr22: 31086034 | 22 | 31086034 | C/T | 0.4868 | 0.5 | 0.375 | 0.125 |
| chr22: 35381808 | 22 | 35381808 | C/A | 0.4972 | 0.5 | 0.375 | 0.125 |
| chr22: 36755505 | 22 | 36755505 | T/C | 0.478 | 0.499 | 0.375 | 0.125 |
| chr22: 38682942 | 22 | 38682942 | T/C | 0.4996 | 0.5 | 0.375 | 0.125 |
| chr22: 39367205 | 22 | 39367205 | T/C | 0.4329 | 0.491 | 0.37 | 0.121 |
| chr22: 41413753 | 22 | 41413753 | A/G | 0.4896 | 0.5 | 0.375 | 0.125 |
| chr22: 44998145 | 22 | 44998145 | T/C | 0.4996 | 0.5 | 0.375 | 0.125 |
| chr22: 47307004 | 22 | 47307004 | G/C | 0.5 | 0.5 | 0.375 | 0.125 |
| chr22: 49463695 | 22 | 49463695 | T/C | 0.4189 | 0.487 | 0.368 | 0.119 |
| chr22: 56554292 | 22 | 56554292 | T/C | 0.4984 | 0.5 | 0.375 | 0.125 |
| chr22: 61422322 | 22 | 61422322 | G/A | 0.484 | 0.5 | 0.375 | 0.125 |
| chr23: 737731 | 23 | 737731 | A/G | 0.4269 | 0.49 | 0.37 | 0.12 |
| BovineHD2300001017 | 23 | 4401397 | T/C | 0.4832 | 0.5 | 0.375 | 0.125 |
| BovineHD2300001206 | 23 | 5077287 | C/T | 0.4836 | 0.5 | 0.375 | 0.125 |
| chr23: 7313850 | 23 | 7313850 | C/A | 0.4984 | 0.5 | 0.375 | 0.125 |
| chr23: 9586831 | 23 | 9586831 | T/A | 0.4996 | 0.5 | 0.375 | 0.125 |
| chr23: 12553957 | 23 | 12553957 | G/A | 0.4996 | 0.5 | 0.375 | 0.125 |
| ARS-BFGL-NGS-78514 | 23 | 14313819 | C/T | 0.4908 | 0.5 | 0.375 | 0.125 |
| chr23: 16538832 | 23 | 16538832 | A/G | 0.4948 | 0.5 | 0.375 | 0.125 |
| chr23: 18262499 | 23 | 18262499 | C/T | 0.4772 | 0.499 | 0.374 | 0.124 |
| chr23: 20970270 | 23 | 20970270 | A/G | 0.4904 | 0.5 | 0.375 | 0.125 |
| chr23: 22621637 | 23 | 22621637 | A/G | 0.4932 | 0.5 | 0.375 | 0.125 |
| chr23: 24292015 | 23 | 24292015 | G/T | 0.4533 | 0.496 | 0.373 | 0.123 |
| chr23: 25670252 | 23 | 25670252 | G/A | 0.3854 | 0.474 | 0.362 | 0.112 |
| chr23: 27655408 | 23 | 27655408 | T/G | 0.4944 | 0.5 | 0.375 | 0.125 |
| chr23: 30553540 | 23 | 30553540 | T/C | 0.4944 | 0.5 | 0.375 | 0.125 |
| chr23: 32360632 | 23 | 32360632 | T/C | 0.4625 | 0.497 | 0.374 | 0.124 |
| chr23: 34492864 | 23 | 34492864 | A/T | 0.494 | 0.5 | 0.375 | 0.125 |
| chr23: 36557706 | 23 | 36557706 | T/C | 0.4848 | 0.5 | 0.375 | 0.125 |
| chr23: 39647590 | 23 | 39647590 | C/T | 0.4896 | 0.5 | 0.375 | 0.125 |
| chr23: 41416362 | 23 | 41416362 | A/G | 0.4796 | 0.499 | 0.375 | 0.125 |
| chr23: 43316240 | 23 | 43316240 | G/A | 0.4213 | 0.488 | 0.369 | 0.119 |
| chr23: 45422704 | 23 | 45422704 | C/T | 0.4992 | 0.5 | 0.375 | 0.125 |
| chr23: 47896708 | 23 | 47896708 | C/T | 0.4992 | 0.5 | 0.375 | 0.125 |
| chr23: 50385911 | 23 | 50385911 | G/T | 0.4317 | 0.491 | 0.37 | 0.12 |
| chr23: 52503653 | 23 | 52503653 | G/A | 0.4429 | 0.494 | 0.372 | 0.122 |
| chr24: 1498986 | 24 | 1498986 | C/T | 0.4932 | 0.5 | 0.375 | 0.125 |
| BovineHD2400001782 | 24 | 6635248 | T/C | 0.4908 | 0.5 | 0.375 | 0.125 |
| BovineHD2400002974 | 24 | 10461518 | T/G | 0.4692 | 0.498 | 0.374 | 0.124 |
| BovineHD2400003128 | 24 | 11043284 | G/T | 0.4776 | 0.499 | 0.374 | 0.125 |
| chr24: 12213557 | 24 | 12213557 | G/A | 0.4984 | 0.5 | 0.375 | 0.125 |
| chr24: 14917351 | 24 | 14917351 | C/A | 0.4992 | 0.5 | 0.375 | 0.125 |
| chr24: 17169312 | 24 | 17169312 | T/C | 0.4996 | 0.5 | 0.375 | 0.125 |
| chr24: 19292120 | 24 | 19292120 | G/C | 0.498 | 0.5 | 0.375 | 0.125 |
| chr24: 21820599 | 24 | 21820599 | T/C | 0.474 | 0.499 | 0.374 | 0.124 |
| chr24: 23901873 | 24 | 23901873 | T/C | 0.4888 | 0.5 | 0.375 | 0.125 |
| chr24: 26177933 | 24 | 26177933 | C/T | 0.4201 | 0.487 | 0.369 | 0.119 |
| chr24: 27712396 | 24 | 27712396 | T/A | 0.4545 | 0.496 | 0.373 | 0.123 |
| chr24: 30518675 | 24 | 30518675 | C/G | 0.4972 | 0.5 | 0.375 | 0.125 |
| chr24: 34261251 | 24 | 34261251 | C/T | 0.4728 | 0.499 | 0.374 | 0.124 |
| chr24: 36738426 | 24 | 36738426 | C/A | 0.4996 | 0.5 | 0.375 | 0.125 |
| chr24: 39303552 | 24 | 39303552 | G/A | 0.498 | 0.5 | 0.375 | 0.125 |
| chr24: 42418587 | 24 | 42418587 | T/A | 0.4856 | 0.5 | 0.375 | 0.125 |
| chr24: 44987204 | 24 | 44987204 | T/C | 0.4904 | 0.5 | 0.375 | 0.125 |
| chr24: 47032284 | 24 | 47032284 | A/C | 0.5 | 0.5 | 0.375 | 0.125 |
| chr24: 49816564 | 24 | 49816564 | C/A | 0.4984 | 0.5 | 0.375 | 0.125 |
| BovineHD2400014878 | 24 | 52567761 | T/C | 0.498 | 0.5 | 0.375 | 0.125 |
| chr24: 54141039 | 24 | 54141039 | T/G | 0.4868 | 0.5 | 0.375 | 0.125 |
| chr24: 57719811 | 24 | 57719811 | A/G | 0.4513 | 0.495 | 0.373 | 0.123 |
| chr24: 59845272 | 24 | 59845272 | C/T | 0.4832 | 0.5 | 0.375 | 0.125 |
| chr24: 62616509 | 24 | 62616509 | T/C | 0.4988 | 0.5 | 0.375 | 0.125 |
| BovineHD2500000375 | 25 | 1817313 | C/T | 0.4824 | 0.5 | 0.375 | 0.125 |
| chr25: 2270319 | 25 | 2270319 | C/A | 0.4904 | 0.5 | 0.375 | 0.125 |
| BovineHD2500000754 | 25 | 3526794 | A/C | 0.4996 | 0.5 | 0.375 | 0.125 |
| chr25: 5235133 | 25 | 5235133 | A/G | 0.4848 | 0.5 | 0.375 | 0.125 |
| chr25: 7435898 | 25 | 7435898 | C/A | 0.4988 | 0.5 | 0.375 | 0.125 |
| chr25: 9485224 | 25 | 9485224 | C/T | 0.4856 | 0.5 | 0.375 | 0.125 |
| chr25: 11983738 | 25 | 11983738 | A/G | 0.4972 | 0.5 | 0.375 | 0.125 |
| chr25: 14309813 | 25 | 14309813 | A/G | 0.4888 | 0.5 | 0.375 | 0.125 |
| chr25: 16824007 | 25 | 16824007 | G/C | 0.498 | 0.5 | 0.375 | 0.125 |
| chr25: 20265328 | 25 | 20265328 | A/T | 0.498 | 0.5 | 0.375 | 0.125 |
| chr25: 23776686 | 25 | 23776686 | A/T | 0.5 | 0.5 | 0.375 | 0.125 |
| chr25: 25356383 | 25 | 25356383 | G/A | 0.476 | 0.499 | 0.374 | 0.124 |
| BovineHD2500007701 | 25 | 27570361 | A/G | 0.4988 | 0.5 | 0.375 | 0.125 |
| BovineHD2500008282 | 25 | 29754420 | A/G | 0.4996 | 0.5 | 0.375 | 0.125 |
| chr25: 31426099 | 25 | 31426099 | C/T | 0.4932 | 0.5 | 0.375 | 0.125 |
| chr25: 33447705 | 25 | 33447705 | G/A | 0.4992 | 0.5 | 0.375 | 0.125 |
| chr25: 35354192 | 25 | 35354192 | G/A | 0.4928 | 0.5 | 0.375 | 0.125 |
| chr25: 37706882 | 25 | 37706882 | C/T | 0.4948 | 0.5 | 0.375 | 0.125 |
| chr25: 39617743 | 25 | 39617743 | T/C | 0.4996 | 0.5 | 0.375 | 0.125 |
| chr25: 41421831 | 25 | 41421831 | G/C | 0.4816 | 0.5 | 0.375 | 0.125 |
| BovineHD2600000537 | 26 | 3159275 | A/G | 0.4976 | 0.5 | 0.375 | 0.125 |
| BovineHD2600001618 | 26 | 7064821 | T/G | 0.4764 | 0.499 | 0.374 | 0.124 |
| chr26: 8295838 | 26 | 8295838 | T/A | 0.4988 | 0.5 | 0.375 | 0.125 |
| chr26: 10496908 | 26 | 10496908 | A/G | 0.492 | 0.5 | 0.375 | 0.125 |
| chr26: 12492633 | 26 | 12492633 | G/C | 0.4968 | 0.5 | 0.375 | 0.125 |
| chr26: 15552507 | 26 | 15552507 | A/T | 0.4637 | 0.498 | 0.374 | 0.124 |
| chr26: 18648652 | 26 | 18648652 | G/A | 0.4984 | 0.5 | 0.375 | 0.125 |
| chr26: 20477934 | 26 | 20477934 | G/A | 0.4996 | 0.5 | 0.375 | 0.125 |
| chr26: 23396729 | 26 | 23396729 | C/T | 0.5 | 0.5 | 0.375 | 0.125 |
| chr26: 27384764 | 26 | 27384764 | A/C | 0.4924 | 0.5 | 0.375 | 0.125 |
| chr26: 30073565 | 26 | 30073565 | A/G | 0.4936 | 0.5 | 0.375 | 0.125 |
| chr26: 32876281 | 26 | 32876281 | A/T | 0.4992 | 0.5 | 0.375 | 0.125 |
| chr26: 36469097 | 26 | 36469097 | G/A | 0.4996 | 0.5 | 0.375 | 0.125 |
| BovineHD2600010617 | 26 | 38760186 | A/G | 0.5 | 0.5 | 0.375 | 0.125 |
| chr26: 40450911 | 26 | 40450911 | T/G | 0.5 | 0.5 | 0.375 | 0.125 |
| chr26: 44726125 | 26 | 44726125 | T/C | 0.5 | 0.5 | 0.375 | 0.125 |
| chr26: 46922819 | 26 | 46922819 | C/T | 0.4976 | 0.5 | 0.375 | 0.125 |
| chr26: 50226218 | 26 | 50226218 | G/A | 0.5 | 0.5 | 0.375 | 0.125 |
| chr27: 103736 | 27 | 103736 | C/T | 0.4908 | 0.5 | 0.375 | 0.125 |
| chr27: 1101354 | 27 | 1101354 | A/G | 0.4916 | 0.5 | 0.375 | 0.125 |
| chr27: 3226775 | 27 | 3226775 | C/A | 0.4764 | 0.499 | 0.374 | 0.124 |
| chr27: 5017483 | 27 | 5017483 | T/C | 0.4992 | 0.5 | 0.375 | 0.125 |
| BovineHD2700002543 | 27 | 8093753 | A/C | 0.4972 | 0.5 | 0.375 | 0.125 |
| chr27: 8768007 | 27 | 8768007 | T/A | 0.4796 | 0.499 | 0.375 | 0.125 |
| BovineHD2700003358 | 27 | 11305871 | T/C | 0.4988 | 0.5 | 0.375 | 0.125 |
| chr27: 12767506 | 27 | 12767506 | T/C | 0.4992 | 0.5 | 0.375 | 0.125 |
| chr27: 14747630 | 27 | 14747630 | T/A | 0.4752 | 0.499 | 0.374 | 0.124 |
| chr27: 16188798 | 27 | 16188798 | G/A | 0.4888 | 0.5 | 0.375 | 0.125 |
| chr27: 18631002 | 27 | 18631002 | G/A | 0.4988 | 0.5 | 0.375 | 0.125 |
| chr27: 20739697 | 27 | 20739697 | A/T | 0.4764 | 0.499 | 0.374 | 0.124 |
| chr27: 23238215 | 27 | 23238215 | G/A | 0.496 | 0.5 | 0.375 | 0.125 |
| chr27: 25163977 | 27 | 25163977 | A/T | 0.4081 | 0.483 | 0.366 | 0.117 |
| chr27: 27780471 | 27 | 27780471 | A/T | 0.4936 | 0.5 | 0.375 | 0.125 |
| chr27: 29007650 | 27 | 29007650 | C/T | 0.4976 | 0.5 | 0.375 | 0.125 |
| chr27: 32882318 | 27 | 32882318 | G/C | 0.4992 | 0.5 | 0.375 | 0.125 |
| chr27: 34293543 | 27 | 34293543 | C/T | 0.4609 | 0.497 | 0.373 | 0.123 |
| chr27: 37259753 | 27 | 37259753 | T/C | 0.4908 | 0.5 | 0.375 | 0.125 |
| chr27: 39274159 | 27 | 39274159 | C/T | 0.4944 | 0.5 | 0.375 | 0.125 |
| chr27: 41321218 | 27 | 41321218 | A/C | 0.4968 | 0.5 | 0.375 | 0.125 |
| chr28: 140159 | 28 | 140159 | T/A | 0.4812 | 0.499 | 0.375 | 0.125 |
| chr28: 2239999 | 28 | 2239999 | C/T | 0.4996 | 0.5 | 0.375 | 0.125 |
| chr28: 4180876 | 28 | 4180876 | C/G | 0.4984 | 0.5 | 0.375 | 0.125 |
| BovineHD2800001838 | 28 | 6164460 | G/A | 0.4728 | 0.499 | 0.374 | 0.124 |
| chr28: 8465913 | 28 | 8465913 | T/C | 0.498 | 0.5 | 0.375 | 0.125 |
| BovineHD4100018502 | 28 | 9663509 | C/T | 0.4976 | 0.5 | 0.375 | 0.125 |
| BovineHD2800003355 | 28 | 11042073 | T/C | 0.4393 | 0.493 | 0.371 | 0.121 |
| chr28: 13676267 | 28 | 13676267 | T/C | 0.4996 | 0.5 | 0.375 | 0.125 |
| BovineHD2800004610 | 28 | 16557050 | G/A | 0.4884 | 0.5 | 0.375 | 0.125 |
| chr28: 18341653 | 28 | 18341653 | T/A | 0.4752 | 0.499 | 0.374 | 0.124 |
| chr28: 20964219 | 28 | 20964219 | G/T | 0.5 | 0.5 | 0.375 | 0.125 |
| chr28: 22247295 | 28 | 22247295 | A/T | 0.496 | 0.5 | 0.375 | 0.125 |
| chr28: 25042637 | 28 | 25042637 | A/C | 0.4852 | 0.5 | 0.375 | 0.125 |
| chr28: 28278314 | 28 | 28278314 | G/A | 0.4956 | 0.5 | 0.375 | 0.125 |
| chr28: 30097594 | 28 | 30097594 | T/A | 0.4988 | 0.5 | 0.375 | 0.125 |
| chr28: 32561401 | 28 | 32561401 | G/A | 0.4952 | 0.5 | 0.375 | 0.125 |
| BovineHD2800009155 | 28 | 34089913 | C/A | 0.4888 | 0.5 | 0.375 | 0.125 |
| chr28: 36080441 | 28 | 36080441 | T/C | 0.4956 | 0.5 | 0.375 | 0.125 |
| chr28: 38019304 | 28 | 38019304 | C/T | 0.4992 | 0.5 | 0.375 | 0.125 |
| chr28: 40659487 | 28 | 40659487 | G/C | 0.4996 | 0.5 | 0.375 | 0.125 |
| chr28: 42821390 | 28 | 42821390 | C/T | 0.4984 | 0.5 | 0.375 | 0.125 |
| chr28: 44511041 | 28 | 44511041 | C/G | 0.4988 | 0.5 | 0.375 | 0.125 |
| chr28: 45635975 | 28 | 45635975 | C/T | 0.4992 | 0.5 | 0.375 | 0.125 |
| chr29: 1772654 | 29 | 1772654 | A/G | 0.4836 | 0.5 | 0.375 | 0.125 |
| chr29: 3970906 | 29 | 3970906 | G/A | 0.498 | 0.5 | 0.375 | 0.125 |
| BovineHD2900002113 | 29 | 7484023 | A/C | 0.4653 | 0.498 | 0.374 | 0.124 |
| chr29: 9604215 | 29 | 9604215 | G/C | 0.4968 | 0.5 | 0.375 | 0.125 |
| chr29: 11737448 | 29 | 11737448 | A/G | 0.4968 | 0.5 | 0.375 | 0.125 |
| chr29: 14120989 | 29 | 14120989 | A/G | 0.4968 | 0.5 | 0.375 | 0.125 |
| BovineHD2900004975 | 29 | 16675393 | G/A | 0.486 | 0.5 | 0.375 | 0.125 |
| chr29: 19524544 | 29 | 19524544 | T/G | 0.4976 | 0.5 | 0.375 | 0.125 |
| chr29: 21310589 | 29 | 21310589 | C/G | 0.4864 | 0.5 | 0.375 | 0.125 |
| chr29: 23512363 | 29 | 23512363 | A/C | 0.4992 | 0.5 | 0.375 | 0.125 |
| chr29: 25651720 | 29 | 25651720 | A/G | 0.4996 | 0.5 | 0.375 | 0.125 |
| chr29: 29237021 | 29 | 29237021 | C/T | 0.4932 | 0.5 | 0.375 | 0.125 |
| chr29: 31663327 | 29 | 31663327 | A/G | 0.4481 | 0.495 | 0.372 | 0.122 |
| chr29: 34085155 | 29 | 34085155 | G/A | 0.4996 | 0.5 | 0.375 | 0.125 |
| chr29: 36484686 | 29 | 36484686 | C/A | 0.4984 | 0.5 | 0.375 | 0.125 |
| chr29: 39714340 | 29 | 39714340 | A/T | 0.4844 | 0.5 | 0.375 | 0.125 |
| chr29: 43321959 | 29 | 43321959 | A/G | 0.4988 | 0.5 | 0.375 | 0.125 |
| chr29: 46568838 | 29 | 46568838 | T/G | 0.4988 | 0.5 | 0.375 | 0.125 |
| chr29: 48902564 | 29 | 48902564 | G/A | 0.4888 | 0.5 | 0.375 | 0.125 |
| BovineHD2900014811 | 29 | 50783100 | G/A | 0.4976 | 0.5 | 0.375 | 0.125 |

The present disclosure further provides a probe for identifying the SNP molecular marker combination.

In the present disclosure, there is no special limitation on a design method of the probe, which can be designed using conventional methods in the field, and can be designed to meet specific recognition and binding to the corresponding SNP sites.

The present disclosure further provides a gene chip prepared based on the SNP molecular marker combination.

In the present disclosure, the gene chip preferably includes a liquid chip. The liquid chip can visually determine a genotype of the site corresponding to the SNP molecular marker, thereby facilitating the determination of genetic relationship.

The present disclosure further provides use of the SNP molecular marker combination or the probe or the gene chip in genetic relationship identification of a Huaxi cattle.

In the present disclosure, a genomic DNA of a Huaxi cattle to be tested is used as a template when identifying the genetic relationship of the Huaxi cattle; the genotype of the SNP molecular marker site is determined using conventional means in this field, such as sequencing and gene chip; and paternity testing is conducted based on a likelihood method. Preferably, a logarithm of the odds (LOD) value of a paternity index is calculated based on the genotype of the Huaxi cattle to be tested, a likelihood function is established, and a hypothesis testing is conducted to find the most similar male parent. The process and calculation method are as follows:

The results are obtained according to the calculation formula proposed by Kalinowski et al. (2007): Kalinowski S T, Taper M L, Marshall T C. Revising how the computer program CERVUS accommodates genotyping error increases success in paternity assignment [J]. Molecular ecology, 2007, 16(5): 1099-1106.

(1) a possibility of a hypothetical male parent becoming a true male parent when a maternal genotype is unknown:

$$L ( {H}_{1} ) = P ( {g}_{a} ) \{ {( 1 - ε )}^{2} T ( {g}_{o} \vert {g}_{a} ) + {ε ( 1 - ε )}^{2} P ( {g}_{o} ) + {ε}^{2} P ( {g}_{o} ) \} \\ L ( {H}_{2} ) = P ( {g}_{a} ) \{ {( 1 - ε )}^{2} P ( {g}_{o} ) + {ε ( 1 - ε )}^{2} P ( {g}_{o} ) + {ε}^{2} P ( {g}_{o} ) \}$$

(2) a possibility of a hypothetical male parent becoming a true male parent when a maternal genotype is known:

$$L ( {H}_{1} ) = P ( {g}_{m} ) P ( {g}_{a} ) \{ {( 1 - ε )}^{3} T ( {g}_{o} \vert {g}_{m} , {g}_{a} ) + {ε ( 1 - ε )}^{2} [ T ( {g}_{o} \vert {g}_{m} ) + T ( {g}_{o} \vert {g}_{a} ) + P ( {g}_{o} ) ] + {{ε}^{2} ( 1 - ε )}^{3} P ( {g}_{o} ) + {ε}^{3} P ( {g}_{o} ) \} \\ L ( {H}_{2} ) = P ( {g}_{m} ) P ( {g}_{a} ) \{ {( 1 - ε )}^{3} T ( {g}_{o} \vert {g}_{m} ) + {ε ( 1 - ε )}^{2} [ T ( {g}_{o} \vert {g}_{m} ) + P ( {g}_{o} ) ] + {{ε}^{2} ( 1 - ε )}^{3} P ( {g}_{o} ) + {ε}^{3} P ( {g}_{o} ) \} .$$

(3) a possibility of a hypothetical female parent and a hypothetical male parent becoming parents of an offspring individual:

$$L ( {H}_{1} ) = P ( {g}_{am} ) P ( {g}_{a} ) \{ {( 1 - ε )}^{3} T ( {g}_{o} \vert {g}_{am} , {g}_{a} ) + {ε ( 1 - ε )}^{2} [ T ( {g}_{o} \vert {g}_{am} ) + T ( {g}_{o} \vert {g}_{a} ) + P ( {g}_{o} ) ] + {{ε}^{2} ( 1 - ε )}^{3} P ( {g}_{o} ) + {ε}^{3} P ( {g}_{o} ) \} \\ L ( {H}_{2} ) = P ( {g}_{ma} ) P ( {g}_{a} ) \{ {( 1 - ε )}^{3} P ( {g}_{o} ) + {ε ( 1 - ε )}^{2} P ( {g}_{o} ) + {{ε}^{2} ( 1 - ε )}^{3} P ( {g}_{o} ) + {ε}^{3} P ( {g}_{o} ) \} .$$

(4) an expression formula of a likelihood ratio is: L(H₁)/L(H₂), then:

$$LOD = ln [ L ( {H}_{1} ) / L ( {H}_{2} ) ]$$

In the above formulas, H₁: hypothetical male parent is a true male parent, H₂: hypothetical male parent is an unrelated individual. L(H₁) and L(H₂) represent likelihood functions under hypothetical conditions H. gₒ represents a genotype of the offspring, gₐ represents a genotype of the hypothetical male parent, gₘ represents a genotype of the known female parent, gₐₘ represents a genotype of the hypothetical female parent, T represents a standard Mendelian transmission probability, P represents a genotype probability, and ε represents a genotype discrimination error rate.

According to the above calculation formulas, the LOD value of the likelihood ratio of each candidate parent at all detection sites can be obtained, and the possibility that the candidate parent is the true parent of the offspring can be known, such that the genetic relationship between the parent-child pair can be determined.

Marshall et al. have also pointed out based on the definition of LOD value that LOD value equal to 0 means that the hypothetical male parent has the same probability of becoming the true male parent of the offspring as a random male individual in the population. An LOD value less than 0 means that the hypothetical male parent cannot be the true male parent of the offspring, which generally means that there is a genotype mismatch at one or more loci between the hypothetical male parent and the offspring. When LOD is greater than 0, it means that the hypothetical male parent is more likely to be the true parent of the offspring. When the LOD value is high enough, the genetic relationship between the offspring and the candidate male parent can be determined.

When the LOD values calculated by multiple candidate parents are all greater than 0, then they may all become the true male parents of the offspring, but there is actually only one true male parent. At this time, the candidate parents can be lined up according to the size of the LOD value. The one with a higher LOD value is more likely to become the true male parent. However, sometimes there may be some positive LOD values that are approximately equal, making the determination difficult. In view of this situation, Marshall et al. (Marshall T C, Slate J, Kruuk L E B, et al. Statistical confidence for likelihood-based paternity inference in natural populations [J]. Molecular ecology, 1998, 7(5): 639-655.) have defined a statistic Δ for paternity testing:

$$Δ = {LOD}_{max} - {LOD}_{sec}$$

Δ is the statistic to evaluate the credibility of the identification results. LODₘₐₓ represents the LOD value of the most similar parent, and LOD_{sec} represents the LOD value of the second similar parent. The larger the value of Δ, the higher the confidence that the most similar parent is the true parent. Δ for paternity testing can ensure an accuracy of the identification. Meanwhile, the confidence level of the most similar parent assignment is also be displayed: * indicates that the parent-child relationship is extremely significant, and the confidence level exceeds 95%; + indicates that the parent-child relationship is significant, and the confidence level exceeds 80%; and − indicates that the parent-child relationship does not meet the significant requirements, and the confidence level is 0% to 80%.

Therefore, the method for genetic relationship identification of a Huaxi cattle provided by the present disclosure includes: calculating an LOD value of a paternity index according to the genotype of the Huaxi cattle to be identified; and identifying a genetic relationship of the Huaxi cattle to be identified based on the LOD value. When the LOD value is greater than 0, a candidate parent has a possibility of being a true parent, and an individual with a maximum LOD value is regarded as a most similar parent; and when the LOD value is less than 0, the candidate parent does not have the possibility of being the true parent.

The present disclosure further provides use of the SNP molecular marker combination or the probe or the gene chip in improved genetic breeding of a Huaxi cattle.

In the present disclosure, the SNP molecular marker combination and corresponding products can be used for complete and accurate pedigree identification of the Huaxi cattle, improving a breeding accuracy and accelerating a genetic progress of the Huaxi cattle, and exhibit excellent application prospects and economic benefits.

The present disclosure further provides a method for genetic relationship identification of a Huaxi cattle, including the following steps: identifying a genotype of the SNP molecular marker combination using a genomic DNA of a Huaxi cattle to be identified as a template; calculating an logarithm of the odds (LOD) value of a paternity index according to the genotype of the Huaxi cattle to be identified; and identifying a genetic relationship of the Huaxi cattle to be identified based on the LOD value.

In the present disclosure, a specific LOD calculation method is the same as above and will not be repeated here. Similarly, the LOD value is greater than 0, a candidate parent has a possibility of being a true parent, and an individual with a maximum LOD value is regarded as a most similar parent; and when the LOD value is less than 0, the candidate parent does not have the possibility of being the true parent.

In order to further illustrate the present disclosure, the SNP molecular marker combination for genetic relationship identification of a Huaxi cattle, the use, and the identification method provided by the present disclosure are detail below in connection with accompanying drawings and examples, but these examples should not be understood as limiting the claimed scope of the present disclosure.

Unless otherwise specified, technical means used in the examples are well known to those skilled in the art, and all chemical reagents used are commercially available.

### Example 1

Screening of 1,000 SNP molecular markers related to genetic relationship identification of Huaxi cattle

### 1. Screening of SNP Molecular Markers Related to Genetic Relationship Identification of Huaxi Cattle

Second-generation sequencing was conducted on 1,252 Huaxi cattle from the Ulgai Management Area in Inner Mongolia using the Illumina platform, and a total of 12,468,401 SNP molecular markers were obtained. The following quality control criteria were adopted to select the SNPs: (1) SNP located on an autosome; (2) MAF of greater than 0.35; (3) call rate of each SNP marker of greater than 0.95; (4) a distance between two adjacent SNPs on a same autosome of greater than 1 Mb; and screening conducted on separate chromosomes in order to avoid linkage disequilibrium between the selected SNP molecular markers; (5) Hardy-Weinberg equilibrium test (HW) P>1×10⁶. 17,023 SNP sites were obtained through preliminary screening.

The genetic polymorphism parameters of the 17,023 SNPs were calculated using CERVUS3.0.7 software, including: MAF, HExp, PIC, Hardy-Weinberg equilibrium, average PE of excluding a suspected parent, CPE of site combination, and invalid allele frequency (IAF).

According to the statistical results of each parameter, the chromosomes were selected separately again. The sites were re-sequenced according to PE. According to PIC and referring to MAF, the site with the highest parameter value is retained under the condition that a distance between two adjacent SNPs on a same autosome is greater than 2 Mb. There are eventually 1,000 markers that are selected. The number distribution of SNP sites on each chromosome was shown in Table 2.

Table 2 Number of SNPs on each autosome and distance between two adjacent SNPs

| Chromosome | Number of SNPs | Average distance |
|---|---|---|
| 1 | 73 | 2148861 |
| 2 | 60 | 2240010 |
| 3 | 58 | 2073332 |
| 4 | 54 | 2136631 |
| 5 | 56 | 2152963 |
| 6 | 37 | 3077272 |
| 7 | 40 | 2722233 |
| 8 | 45 | 2360971 |
| 9 | 40 | 2513461 |
| 10 | 45 | 2262732 |
| 11 | 37 | 2759505 |
| 12 | 26 | 3302410 |
| 13 | 24 | 3418445 |
| 14 | 31 | 2707235 |
| 15 | 34 | 2470821 |
| 16 | 38 | 2147451 |
| 17 | 24 | 3034389 |
| 18 | 24 | 2063845 |
| 19 | 29 | 2077107 |
| 20 | 24 | 2780808 |
| 21 | 26 | 2618773 |
| 22 | 23 | 2450545 |
| 23 | 25 | 2070637 |
| 24 | 25 | 2444701 |
| 25 | 20 | 1980226 |
| 26 | 18 | 2614830 |
| 27 | 21 | 1962737 |
| 28 | 23 | 1978079 |
| 29 | 20 | 2450522 |

The final SNP molecular marker combination included 1,000 SNP sites shown in Table 1, and the average distance between two adjacent sites on the same autosome was 2 Mb. The average values of MAF, HExp, and PIC of the marker combination were 0.4846, 0.4983, and 0.3741, respectively; the 1,000 SNP molecular markers had a CPE of 0.9999999999999, making paternity testing extremely efficient.

### 2. Establishment of a System for Genetic Relationship Identification of Huaxi Cattle Based on SNP Markers

The greater the number of SNP markers, the higher the accuracy of identifying their genetic relationships. The detection costs varied depending on the number of SNP markers. Since in different actual productions, operational difficulty and inspection costs might vary greatly. In order to meet the needs of various situations, the highest paternity test accuracy was achieved at the most reasonable cost. In this example, the minimum number of SNPs in the combination required to achieve ideal identification effectiveness under different circumstances was finally determined through simulation studies and verification in the Huaxi cattle population.

1,000 SNP markers were randomly divided into sub-combinations containing 500, 300, 200, 100, 80, 70, 60, 50, 40, 30, and 20 to determine the minimum number of SNP markers required under different circumstances. A simulation experiment of paternity testing was conducted using CERVUS3.0.7 software to determine the most suitable SNP combination.

The simulation parameter settings included: a site typing success rate of 1, an analysis error rate of 0.01, critical values of confidence at 80% and 95%, a number of simulated offspring of 10,000, and a candidate parent detection rate of 100%. Statistical analysis of the simulation results (Table 3) found that CPE1 increased from 0.93051929 (20 SNPs) to 1 (200 SNPs), CPE2 increased from 0.98424600 (20 SNPs) to 1 (100 SNPs to 200 SNPs), and CPE3 increased from 0.998644213 (20 SNPs) to 1 (60 SNPs to 200 SNPs). When the site increased to a certain extent, the exclusion rate remained unchanged and reached a maximum of 1. As shown in Table 3, when the number of SNP markers reached 70, both CPE1 and CPE2 exceeded 99.99%, and CPE3 reached 1. The allocation rate of paternity testing (80% and 95% confidence levels) of not less than 70 SNP markers could reach 100%. Moreover, at a paternity confidence level of 95%, the proportion of paternity inference could reach 100%.

Therefore, simulation studies showed that in order to ensure ideal identification efficiency, at least 70 sites were required to form a marker combination in practical applications. This study finally selected 1,000 SNP markers to form the SNP marker combination for genetic relationship identification in the Huaxi cattle population (Table 1).

TABLE 3
PE, allocation rate, and inference rate of different gradient combinations

| | | | | 80% confidence level | | 95% confidence level | |
|---|---|---|---|---|---|---|---|
| Number | | | | Allocation | Inference | Allocation | Inference |
| of SNPs | CPE1 | CPE2 | CPE3 | rate | rate | rate | rate |
| 20 | 0.93051929 | 0.98424600 | 0.99864213 | 15% | 80% | 2% | 95% |
| 30 | 0.98125744 | 0.99799699 | 0.99994905 | 100% | 83% | 69% | 95% |
| 40 | 0.99504842 | 0.99974818 | 0.99999812 | 100% | 97% | 100% | 97% |
| 50 | 0.99864010 | 0.99996766 | 0.99999993 | 100% | 99% | 100% | 99% |
| 60 | 0.99965263 | 0.99999601 | 1 | 100% | 100% | 100% | 100% |
| 70 | 0.99990483 | 0.99999949 | 1 | 100% | 100% | 100% | 100% |
| 80 | 0.99997562 | 0.99999994 | 1 | 100% | 100% | 100% | 100% |
| 100 | 0.99999827 | 1 | 1 | 100% | 100% | 100% | 100% |
| 200 | 1 | 1 | 1 | 100% | 100% | 100% | 100% |
| 300 | 1 | 1 | 1 | 100% | 100% | 100% | 100% |
| 500 | 1 | 1 | 1 | 100% | 100% | 100% | 100% |

Notes:
CPE1 represented the CPE of a suspected parent; CPE2 represented the CPE of a suspected parent when the genotype of the other parent was known; and CPE3 represented the CPE of suspected parent pairs. The allocation rate was the percentage of simulated offspring to which a parent was assigned at this confidence level, and the inference rate was the probability that the assigned parent was the true parent at this confidence level.

### Example 2

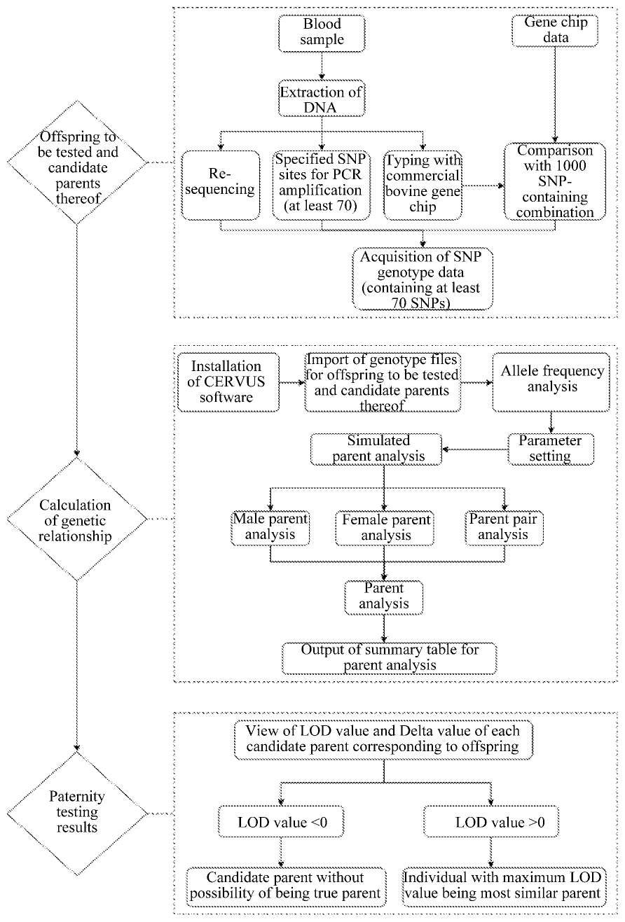

The application of 1,000 SNP molecular markers shown in Table 1 in the genetic relationship identification of Huaxi cattle was shown in FIG. 1:

In order to verify the feasibility and accuracy of the selected 1,000 SNP markers for inferring paternity of Huaxi cattle in actual populations, 10 father-son pairs were selected from the Huaxi cattle population in the Ulgai Management Area of Inner Mongolia using next-generation sequencing based on the Illumina platform. The combination with clear relationship was used for verification test of paternity testing. The candidate male parents of these 10 offspring cattle (numbered A1 to A10) were set to the 14 breeding bulls (numbered P1 to P14) that had been used for breeding in this experimental group. In order to ensure the reliability of the test results, three repeated tests were conducted, each time 70 sites were randomly selected from 1,000 SNP markers to form an identification combination.

The genetic relationship was inferred using CERVUS3.0.7 software, and the results were shown in Tables 4 to 13. Specifically, taking the first test results of the A1 individual as an example, the LOD values of the 14 candidate male parents were arranged from high to low. P1 had the highest LOD value of 15.27655587, Δ value was 15.27655587, and the confidence level exceeded 95%. Therefore, P1 was the most similar parent of A1, and the LOD values of other candidate male parents were negative, indicating that the candidate parents P2 to P14 could not be the true parents of A1.

The most likely male parent was found using a combination of 1,000 SNP molecular markers in the present disclosure. The results verified by three tests were consistent with the pedigree records, and the confidence level reached 95%. This verified the accuracy and feasibility of the SNP molecular marker combination provided by the present disclosure in paternity testing of Huaxi cattle.

TABLE 4
Verification tests for paternity testing

| | Candidate male | Number of | Number of | | | Confidence |
|---|---|---|---|---|---|---|
| Offspring SN | parent SN | typing sites | mismatched sites | LOD value | Δ | coefficient |
| A1 (first time) | P1 | 70 | 0 | 15.27655587 | 15.27655587 | * |
| | P3 | 70 | 3 | −0.798560943 | 0 | |
| | P4 | 70 | 3 | −4.219138941 | 0 | |
| | P10 | 70 | 6 | −16.17091889 | 0 | |
| | P12 | 70 | 6 | −19.89487249 | 0 | |
| | P14 | 70 | 8 | −22.81955294 | 0 | |
| | P6 | 70 | 8 | −26.61632679 | 0 | |
| | P5 | 70 | 8 | −27.22352225 | 0 | |
| | P13 | 70 | 8 | −30.95873714 | 0 | |
| | P7 | 70 | 11 | −36.9207947 | 0 | |
| | P9 | 70 | 12 | −39.02607448 | 0 | |
| | P8 | 70 | 12 | −46.9634922 | 0 | |
| | P2 | 70 | 14 | −52.62278889 | 0 | |
| | P11 | 70 | 15 | −56.54278087 | 0 | |
| A1 (second time) | P1 | 70 | 0 | 20.33895066 | 9.422833615 | * |
| | P3 | 70 | 2 | 10.91611705 | 0 | |
| | P13 | 70 | 6 | −18.61527279 | 0 | |
| | P14 | 70 | 6 | −20.67610141 | 0 | |
| | P9 | 70 | 8 | −23.82143733 | 0 | |
| | P8 | 70 | 8 | −27.3751137 | 0 | |
| | P10 | 70 | 9 | −30.68782123 | 0 | |
| | P4 | 70 | 9 | −30.83593178 | 0 | |
| | P5 | 70 | 11 | −36.88888598 | 0 | |
| | P6 | 70 | 11 | −37.33670367 | 0 | |
| | P7 | 70 | 11 | −38.21948199 | 0 | |
| | P12 | 70 | 12 | −38.22760624 | 0 | |
| | P11 | 70 | 12 | −41.70389841 | 0 | |
| | P2 | 70 | 15 | −52.52303043 | 0 | |
| A1 (third time) | P1 | 70 | 0 | 20.0790786 | 18.32266489 | * |
| | P3 | 70 | 7 | −13.68776522 | 0 | |
| | P8 | 70 | 7 | −22.76529349 | 0 | |
| | P4 | 70 | 8 | −25.14625495 | 0 | |
| | P12 | 70 | 7 | −25.89087463 | 0 | |
| | P10 | 70 | 8 | −25.98556226 | 0 | |
| | P13 | 70 | 8 | −28.65862731 | 0 | |
| | P9 | 70 | 9 | −29.42333074 | 0 | |
| | P5 | 70 | 8 | −30.29730542 | 0 | |
| | P14 | 70 | 10 | −34.14889145 | 0 | |
| | P11 | 70 | 11 | −37.25445637 | 0 | |
| | P7 | 70 | 12 | −37.78814885 | 0 | |
| | P2 | 70 | 12 | −42.88758843 | 0 | |
| | P6 | 70 | 13 | −45.13558649 | 0 | |

Notes:
* indicated that the parent-child relationship was extremely significant, and the confidence level exceeded 95%; + indicated that the parent-child relationship was significant, and the confidence level was 80% to 95%; and − indicated that the parent-child relationship did not meet the significant requirements, and the confidence level was 0% to 80%; the same below.

TABLE 5
Verification tests for paternity testing

| | Candidate male | Number of | Number of | | | Confidence |
|---|---|---|---|---|---|---|
| Offspring SN | parent SN | typing sites | mismatched sites | LOD value | Δ | coefficient |
| A2 (first time) | P2 | 70 | 0 | 13.18418357 | 13.18418357 | * |
| | P8 | 70 | 3 | −6.959785923 | 0 | |
| | P1 | 70 | 4 | −10.91626043 | 0 | |
| | P14 | 70 | 5 | −12.32488352 | 0 | |
| | P13 | 70 | 5 | −13.16474347 | 0 | |
| | P4 | 70 | 5 | −15.26754307 | 0 | |
| | P7 | 70 | 7 | −19.81454203 | 0 | |
| | P3 | 70 | 7 | −23.9442168 | 0 | |
| | P12 | 70 | 7 | −24.52800265 | 0 | |
| | P5 | 70 | 8 | −25.08493021 | 0 | |
| | P10 | 70 | 8 | −27.0238048 | 0 | |
| | P11 | 70 | 8 | −27.37101623 | 0 | |
| | P9 | 70 | 9 | −28.25617077 | 0 | |
| | P6 | 70 | 10 | −35.04615087 | 0 | |
| A2 (second time) | P2 | 70 | 0 | 11.29577665 | 11.29577665 | * |
| | P13 | 70 | 4 | −9.346280482 | 0 | |
| | P1 | 70 | 7 | −18.01053582 | 0 | |
| | P8 | 70 | 6 | −18.25103484 | 0 | |
| | P9 | 70 | 7 | −20.37004582 | 0 | |
| | P14 | 70 | 7 | −23.48077567 | 0 | |
| | P5 | 70 | 7 | −23.5775523 | 0 | |
| | P11 | 70 | 8 | −24.16378733 | 0 | |
| | P10 | 70 | 7 | −25.58293392 | 0 | |
| | P4 | 70 | 7 | −25.71061396 | 0 | |
| | P7 | 70 | 10 | −34.99878274 | 0 | |
| | P6 | 70 | 10 | −35.32377759 | 0 | |
| | P3 | 70 | 10 | −35.35065368 | 0 | |
| | P12 | 70 | 11 | −40.56669855 | 0 | |
| A2 (third time) | P2 | 70 | 0 | 13.67045517 | 13.67045517 | * |
| | P12 | 70 | 5 | −12.63073863 | 0 | |
| | P14 | 70 | 6 | −19.31850583 | 0 | |
| | P6 | 70 | 8 | −25.71908272 | 0 | |
| | P5 | 70 | 9 | −30.04343596 | 0 | |
| | P8 | 70 | 9 | −31.48627584 | 0 | |
| | P10 | 70 | 10 | −32.65961118 | 0 | |
| | P11 | 70 | 10 | −33.96354381 | 0 | |
| | P13 | 70 | 10 | −35.44348416 | 0 | |
| | P9 | 70 | 10 | −35.52689678 | 0 | |
| | P4 | 70 | 12 | −44.70627337 | 0 | |
| | P7 | 70 | 14 | −50.68697135 | 0 | |
| | P3 | 70 | 15 | −55.74985647 | 0 | |
| | P1 | 70 | 15 | −56.40950178 | 0 | |

TABLE 6
Verification tests for paternity testing

| | Candidate male | Number of | Number of | | | Confidence |
|---|---|---|---|---|---|---|
| Offspring SN | parent SN | typing sites | mismatched sites | LOD value | Δ | coefficient |
| A3 (first time) | P3 | 70 | 0 | 17.65817152 | 15.83923359 | * |
| | P4 | 70 | 2 | 1.818937929 | 0 | |
| | P1 | 70 | 5 | −3.72319063 | 0 | |
| | P14 | 70 | 7 | −16.45570595 | 0 | |
| | P5 | 70 | 8 | −22.95432088 | 0 | |
| | P12 | 70 | 8 | −27.377855 | 0 | |
| | P7 | 70 | 11 | −36.61320131 | 0 | |
| | P10 | 70 | 11 | −37.61678853 | 0 | |
| | P8 | 70 | 11 | −39.22610042 | 0 | |
| | P6 | 70 | 12 | −40.73197437 | 0 | |
| | P13 | 70 | 13 | 47.48823692 | 0 | |
| | P9 | 70 | 15 | −52.40864125 | 0 | |
| | P11 | 70 | 14 | 52.62565956 | 0 | |
| | P2 | 70 | 15 | −52.93845129 | 0 | |
| A3 (second time) | P3 | 70 | 0 | 20.73255736 | 11.97081215 | * |
| | P1 | 70 | 2 | 8.761745205 | 0 | |
| | P14 | 70 | 6 | −17.95055839 | 0 | |
| | P4 | 70 | 7 | −23.18036261 | 0 | |
| | P13 | 70 | 8 | −27.64057013 | 0 | |
| | P6 | 70 | 10 | −32.41541512 | 0 | |
| | P10 | 70 | 10 | −35.41577746 | 0 | |
| | P2 | 70 | 11 | −36.82857075 | 0 | |
| | P9 | 70 | 11 | −37.77354431 | 0 | |
| | P5 | 70 | 11 | −38.15154632 | 0 | |
| | P8 | 70 | 11 | −41.41793524 | 0 | |
| | P11 | 70 | 13 | −45.72789696 | 0 | |
| | P12 | 70 | 14 | −49.13629215 | 0 | |
| | P7 | 70 | 14 | −51.94051363 | 0 | |
| A3 (third time) | P3 | 70 | 0 | 19.23732995 | 18.51024025 | * |
| | P1 | 70 | 6 | −10.27358893 | 0 | |
| | P13 | 70 | 7 | 22.80168087 | 0 | |
| | P14 | 70 | 7 | −23.449311 | 0 | |
| | P11 | 70 | 7 | 23.7404873 | 0 | |
| | P4 | 70 | 8 | −24.88806342 | 0 | |
| | P9 | 70 | 8 | −25.1518484 | 0 | |
| | P8 | 70 | 9 | −27.0157207 | 0 | |
| | P5 | 70 | 8 | −27.18924531 | 0 | |
| | P12 | 70 | 9 | −30.71930002 | 0 | |
| | P6 | 70 | 11 | −37.66082461 | 0 | |
| | P10 | 70 | 11 | −38.02837412 | 0 | |
| | P7 | 70 | 13 | −42.82485878 | 0 | |
| | P2 | 70 | 12 | −44.57026097 | 0 | |

TABLE 7
Verification tests for paternity testing

| | Candidate male | Number of | Number of | | | Confidence |
|---|---|---|---|---|---|---|
| Offspring SN | parent SN | typing sites | mismatched sites | LOD value | Δ | coefficient |
| A4 (first time) | P4 | 70 | 1 | 4.447875412 | 4.447875412 | * |
| | P6 | 70 | 4 | −7.234242281 | 0 | |
| | P11 | 70 | 4 | −10.39769656 | 0 | |
| | P3 | 70 | 6 | −16.39390576 | 0 | |
| | P8 | 70 | 5 | −17.57537758 | 0 | |
| | P12 | 70 | 6 | −18.1692707 | 0 | |
| | P2 | 70 | 7 | −22.36874143 | 0 | |
| | P1 | 70 | 8 | −22.54132755 | 0 | |
| | P10 | 70 | 8 | −25.46248811 | 0 | |
| | P7 | 70 | 8 | −26.13169774 | 0 | |
| | P5 | 70 | 9 | −28.54008271 | 0 | |
| | P14 | 70 | 8 | −28.54804304 | 0 | |
| | P9 | 70 | 9 | −30.28189271 | 0 | |
| | P13 | 70 | 12 | −45.84256129 | 0 | |
| A4 (second time) | P4 | 70 | 1 | 8.076631593 | 8.076631593 | * |
| | P10 | 70 | 5 | −15.13259516 | 0 | |
| | P6 | 70 | 7 | −18.45953574 | 0 | |
| | P9 | 70 | 8 | −23.1248388 | 0 | |
| | P11 | 70 | 9 | −28.24571823 | 0 | |
| | P3 | 70 | 9 | −28.32686404 | 0 | |
| | P12 | 70 | 9 | −29.73527305 | 0 | |
| | P5 | 70 | 9 | −31.50634776 | 0 | |
| | P13 | 70 | 10 | −31.65465875 | 0 | |
| | P14 | 70 | 9 | −32.33394983 | 0 | |
| | P2 | 70 | 11 | −35.86671424 | 0 | |
| | P7 | 70 | 11 | −36.18762834 | 0 | |
| | P8 | 70 | 11 | −39.09735076 | 0 | |
| | P1 | 70 | 13 | −42.83331045 | 0 | |
| A4 (third time) | P4 | 70 | 0 | 11.15166472 | 9.285727602 | * |
| | P13 | 70 | 1 | 1.865937113 | 0 | |
| | P10 | 70 | 4 | −8.450594515 | 0 | |
| | P2 | 70 | 5 | −12.00300514 | 0 | |
| | P14 | 70 | 4 | −15.60968043 | 0 | |
| | P9 | 70 | 5 | −16.47215691 | 0 | |
| | P11 | 70 | 6 | −18.36013713 | 0 | |
| | P12 | 70 | 6 | −19.33540429 | 0 | |
| | P6 | 70 | 9 | −31.73583046 | 0 | |
| | P8 | 70 | 9 | −33.4966112 | 0 | |
| | P7 | 70 | 11 | −36.7035653 | 0 | |
| | P3 | 70 | 11 | −38.25875666 | 0 | |
| | P1 | 70 | 12 | −40.34824853 | 0 | |
| | P5 | 70 | 12 | −46.15780846 | 0 | |

TABLE 8
Verification tests for paternity testing

| | Candidate male | Number of | Number of | | | Confidence |
|---|---|---|---|---|---|---|
| Offspring SN | parent SN | typing sites | mismatched sites | LOD value | Δ | coefficient |
| A5 (first time) | P5 | 70 | 0 | 11.55221854 | 11.55221854 | * |
| | P4 | 70 | 5 | −13.93916171 | 0 | |
| | P12 | 70 | 7 | −24.08655426 | 0 | |
| | P14 | 70 | 8 | −25.09301558 | 0 | |
| | P10 | 70 | 8 | −28.36342523 | 0 | |
| | P8 | 70 | 8 | −28.98681732 | 0 | |
| | P13 | 70 | 9 | −29.22462872 | 0 | |
| | P6 | 70 | 9 | −31.87105554 | 0 | |
| | P3 | 70 | 10 | −35.90560316 | 0 | |
| | P11 | 70 | 11 | −39.12885663 | 0 | |
| | P2 | 70 | 12 | −41.95748369 | 0 | |
| | P1 | 70 | 12 | −42.77436497 | 0 | |
| | P7 | 70 | 13 | −46.20365351 | 0 | |
| | P9 | 70 | 13 | −47.64016772 | 0 | |
| A5 (second time) | P5 | 70 | 0 | 10.93155537 | 10.93155537 | * |
| | P4 | 70 | 3 | −8.559470821 | 0 | |
| | P3 | 70 | 7 | −17.00239307 | 0 | |
| | P8 | 70 | 5 | −17.00997876 | 0 | |
| | P1 | 70 | 7 | −18.27240548 | 0 | |
| | P14 | 70 | 6 | −19.0577365 | 0 | |
| | P13 | 70 | 6 | −22.1292772 | 0 | |
| | P12 | 70 | 7 | −23.63319882 | 0 | |
| | P9 | 70 | 8 | −24.108637 | 0 | |
| | P10 | 70 | 8 | −28.88516353 | 0 | |
| | P7 | 70 | 9 | −30.85961741 | 0 | |
| | P6 | 70 | 9 | −32.58603489 | 0 | |
| | P2 | 70 | 10 | −34.83261681 | 0 | |
| | P11 | 70 | 12 | −41.63753407 | 0 | |
| A5 (third time) | P5 | 70 | 0 | 7.651764002 | 7.651764002 | * |
| | P12 | 70 | 4 | −13.27129069 | 0 | |
| | P6 | 70 | 4 | −13.65824704 | 0 | |
| | P14 | 70 | 4 | −14.27792222 | 0 | |
| | P11 | 70 | 5 | −14.55944781 | 0 | |
| | P9 | 70 | 5 | −18.09734326 | 0 | |
| | P13 | 70 | 5 | −19.15596807 | 0 | |
| | P3 | 70 | 6 | −19.90049798 | 0 | |
| | P10 | 70 | 6 | −22.06005132 | 0 | |
| | P7 | 70 | 8 | −25.02631878 | 0 | |
| | P4 | 70 | 8 | −26.82490903 | 0 | |
| | P2 | 70 | 8 | −27.9959658 | 0 | |
| | P8 | 70 | 8 | −29.75035311 | 0 | |
| | P1 | 70 | 10 | −34.89595444 | 0 | |

TABLE 9
Verification tests for paternity testing

| | Candidate male | Number of | Number of | | | Confidence |
|---|---|---|---|---|---|---|
| Offspring SN | parent SN | typing sites | mismatched sites | LOD value | Δ | coefficient |
| A6 (first time) | P3 | 70 | 0 | 16.31424863 | 13.69975773 | * |
| | P4 | 70 | 3 | −7.371145054 | 0 | |
| | P1 | 70 | 6 | −9.862608409 | 0 | |
| | P10 | 70 | 7 | −20.62521881 | 0 | |
| | P14 | 70 | 8 | −20.9491442 | 0 | |
| | P12 | 70 | 8 | −26.0164155 | 0 | |
| | P5 | 70 | 9 | −27.74035367 | 0 | |
| | P8 | 70 | 9 | −31.39144378 | 0 | |
| | P13 | 70 | 10 | −37.76710371 | 0 | |
| | P7 | 70 | 13 | −45.26087869 | 0 | |
| | P6 | 70 | 13 | −45.86156357 | 0 | |
| | P9 | 70 | 13 | −47.05626417 | 0 | |
| | P11 | 70 | 15 | −55.1075559 | 0 | |
| | P2 | 70 | 17 | −63.42367779 | 0 | |
| A6 (second time) | P3 | 70 | 0 | 19.1223745 | 18.47185928 | * |
| | P1 | 70 | 3 | 0.650515222 | 0 | |
| | P14 | 70 | 5 | −14.77784216 | 0 | |
| | P9 | 70 | 6 | −15.6385782 | 0 | |
| | P4 | 70 | 7 | −22.8035097 | 0 | |
| | P12 | 70 | 8 | −23.0437581 | 0 | |
| | P13 | 70 | 7 | −24.08750295 | 0 | |
| | P8 | 70 | 7 | −24.22026555 | 0 | |
| | P5 | 70 | 8 | −26.89913641 | 0 | |
| | P2 | 70 | 9 | −32.70390242 | 0 | |
| | P10 | 70 | 9 | −34.82284876 | 0 | |
| | P6 | 70 | 11 | −39.76143236 | 0 | |
| | P7 | 70 | 12 | −43.81241265 | 0 | |
| | P11 | 70 | 14 | −52.16845282 | 0 | |
| A6 (third time) | P3 | 70 | 1 | 12.71204085 | 12.71204085 | * |
| | P1 | 70 | 5 | −7.269807078 | 0 | |
| | P13 | 70 | 5 | −16.42018041 | 0 | |
| | P12 | 70 | 6 | −17.39136592 | 0 | |
| | P9 | 70 | 7 | −21.82162494 | 0 | |
| | P5 | 70 | 7 | −23.03132381 | 0 | |
| | P4 | 70 | 8 | −24.08577339 | 0 | |
| | P8 | 70 | 9 | −27.35673945 | 0 | |
| | P10 | 70 | 9 | −30.27793111 | 0 | |
| | P11 | 70 | 10 | −35.61710466 | 0 | |
| | P6 | 70 | 11 | −35.94224478 | 0 | |
| | P14 | 70 | 10 | −36.94505042 | 0 | |
| | P7 | 70 | 14 | −45.10264542 | 0 | |
| | P2 | 70 | 13 | −47.95849409 | 0 | |

TABLE 10
Verification tests for paternity testing

| | Candidate male | Number of | Number of | | | Confidence |
|---|---|---|---|---|---|---|
| Offspring SN | parent SN | typing sites | mismatched sites | LOD value | Δ | coefficient |
| A7 (first time) | P6 | 70 | 0 | 15.2896811 | 15.2896811 | * |
| | P7 | 70 | 7 | −17.41205643 | 0 | |
| | P14 | 70 | 8 | −23.40960491 | 0 | |
| | P13 | 70 | 8 | −27.83962988 | 0 | |
| | P8 | 70 | 8 | −28.82837783 | 0 | |
| | P11 | 70 | 10 | −32.99655168 | 0 | |
| | P4 | 70 | 9 | −33.19469986 | 0 | |
| | P2 | 70 | 10 | −33.38898099 | 0 | |
| | P1 | 70 | 11 | −34.80130196 | 0 | |
| | P12 | 70 | 10 | −36.24259852 | 0 | |
| | P3 | 70 | 11 | −39.32526735 | 0 | |
| | P9 | 70 | 13 | −43.29871002 | 0 | |
| | P5 | 70 | 13 | −49.12753269 | 0 | |
| | P10 | 70 | 15 | −56.18590308 | 0 | |
| A7 (second time) | P6 | 70 | 0 | 11.7970069 | 11.7970069 | * |
| | P14 | 70 | 4 | −10.70902368 | 0 | |
| | P11 | 70 | 5 | −13.03148311 | 0 | |
| | P10 | 70 | 5 | −13.43526493 | 0 | |
| | P3 | 70 | 6 | −16.78522565 | 0 | |
| | P4 | 70 | 6 | −18.9232738 | 0 | |
| | P7 | 70 | 7 | −19.88003447 | 0 | |
| | P1 | 70 | 7 | −21.19917309 | 0 | |
| | P12 | 70 | 7 | −23.13636389 | 0 | |
| | P9 | 70 | 8 | −24.02601428 | 0 | |
| | P5 | 70 | 7 | −24.4514439 | 0 | |
| | P13 | 70 | 8 | −26.07694434 | 0 | |
| | P8 | 70 | 8 | −28.31506629 | 0 | |
| | P2 | 70 | 9 | −30.78718726 | 0 | |
| A7 (third time) | P6 | 70 | 0 | 14.62115201 | 14.62115201 | * |
| | P10 | 70 | 5 | −11.21567624 | 0 | |
| | P2 | 70 | 7 | −18.18647739 | 0 | |
| | P14 | 70 | 7 | −21.07956251 | 0 | |
| | P8 | 70 | 7 | −21.93249154 | 0 | |
| | P13 | 70 | 7 | −23.05755673 | 0 | |
| | P4 | 70 | 9 | −27.52039187 | 0 | |
| | P12 | 70 | 9 | −31.21901906 | 0 | |
| | P11 | 70 | 10 | −33.33703069 | 0 | |
| | P9 | 70 | 10 | −33.53516124 | 0 | |
| | P5 | 70 | 10 | −37.18837849 | 0 | |
| | P7 | 70 | 12 | −37.28057352 | 0 | |
| | P3 | 70 | 14 | −49.19919001 | 0 | |
| | P1 | 70 | 16 | −54.77701802 | 0 | |

TABLE 11
Verification tests for paternity testing

| | Candidate male | Number of | Number of | | | Confidence |
|---|---|---|---|---|---|---|
| Offspring SN | parent SN | typing sites | mismatched sites | LOD value | Δ | coefficient |
| A8 (first time) | P7 | 70 | 0 | 13.10320746 | 13.10320746 | * |
| | P2 | 70 | 4 | −8.956399502 | 0 | |
| | P8 | 70 | 4 | −10.23948827 | 0 | |
| | P9 | 70 | 5 | −11.07601564 | 0 | |
| | P12 | 70 | 5 | −15.40989744 | 0 | |
| | P11 | 70 | 6 | −17.4642562 | 0 | |
| | P13 | 70 | 6 | −17.77248274 | 0 | |
| | P4 | 70 | 5 | −17.85254156 | 0 | |
| | P1 | 70 | 7 | −19.24587582 | 0 | |
| | P6 | 70 | 7 | −19.6448965 | 0 | |
| | P10 | 70 | 7 | −23.4969286 | 0 | |
| | P14 | 70 | 10 | −34.30083024 | 0 | |
| | P5 | 70 | 10 | −36.74612421 | 0 | |
| | P3 | 70 | 11 | −39.98816304 | 0 | |
| A8 (second time) | P7 | 70 | 1 | 7.365015135 | 7.365015135 | * |
| | P13 | 70 | 3 | −1.579783951 | 0 | |
| | P8 | 70 | 5 | −13.00944639 | 0 | |
| | P10 | 70 | 7 | −24.5714825 | 0 | |
| | P14 | 70 | 7 | −26.08217613 | 0 | |
| | P1 | 70 | 9 | −26.70300284 | 0 | |
| | P11 | 70 | 9 | −30.12971363 | 0 | |
| | P6 | 70 | 10 | −31.20134127 | 0 | |
| | P12 | 70 | 10 | −32.19282515 | 0 | |
| | P5 | 70 | 10 | −34.66088627 | 0 | |
| | P3 | 70 | 11 | −35.89936238 | 0 | |
| | P4 | 70 | 11 | −41.29975669 | 0 | |
| | P9 | 70 | 14 | −49.39038553 | 0 | |
| | P2 | 70 | 14 | −49.53079768 | 0 | |
| A8 (third time) | P7 | 70 | 0 | 19.15191948 | 19.15191948 | * |
| | P9 | 70 | 4 | −7.395052497 | 0 | |
| | P4 | 70 | 4 | −9.782748152 | 0 | |
| | P8 | 70 | 5 | −11.3115943 | 0 | |
| | P14 | 70 | 5 | −14.06148357 | 0 | |
| | P11 | 70 | 6 | −17.92732446 | 0 | |
| | P10 | 70 | 7 | −20.78982904 | 0 | |
| | P12 | 70 | 8 | −25.25783887 | 0 | |
| | P1 | 70 | 10 | −27.46345305 | 0 | |
| | P2 | 70 | 10 | −29.35133289 | 0 | |
| | P13 | 70 | 9 | −29.88102583 | 0 | |
| | P3 | 70 | 10 | −30.06052339 | 0 | |
| | P5 | 70 | 9 | −34.17301755 | 0 | |
| | P6 | 70 | 11 | −36.62510603 | 0 | |

TABLE 12
Verification tests for paternity testing

| | Candidate male | Number of | Number of | | | Confidence |
|---|---|---|---|---|---|---|
| Offspring SN | parent SN | typing sites | mismatched sites | LOD value | Δ | coefficient |
| A9 (first time) | P8 | 70 | 0 | 10.27017646 | 9.819801286 | * |
| | P13 | 70 | 2 | 0.450375171 | 0 | |
| | P12 | 70 | 3 | −5.326807144 | 0 | |
| | P7 | 70 | 6 | −19.58953025 | 0 | |
| | P6 | 70 | 8 | −26.95996607 | 0 | |
| | P5 | 70 | 9 | −27.43717105 | 0 | |
| | P11 | 70 | 8 | −28.923148 | 0 | |
| | P9 | 70 | 9 | −33.29007502 | 0 | |
| | P4 | 70 | 10 | −35.58809353 | 0 | |
| | P3 | 70 | 10 | −35.71232159 | 0 | |
| | P10 | 70 | 10 | −35.79964605 | 0 | |
| | P14 | 70 | 10 | −35.8232246 | 0 | |
| | P1 | 70 | 11 | −36.88513713 | 0 | |
| | P2 | 70 | 12 | −41.8278971 | 0 | |
| A9 (second time) | P8 | 70 | 0 | 8.711390253 | 8.711390253 | * |
| | P14 | 70 | 4 | −10.84782087 | 0 | |
| | P13 | 70 | 5 | −12.80277026 | 0 | |
| | P12 | 70 | 6 | −14.38907764 | 0 | |
| | P9 | 70 | 7 | −21.18470542 | 0 | |
| | P7 | 70 | 8 | −24.18278972 | 0 | |
| | P2 | 70 | 8 | −25.19889071 | 0 | |
| | P6 | 70 | 8 | −25.48349763 | 0 | |
| | P4 | 70 | 8 | −26.93246427 | 0 | |
| | P10 | 70 | 8 | −27.5707651 | 0 | |
| | P11 | 70 | 9 | −29.79477172 | 0 | |
| | P5 | 70 | 9 | −30.50062336 | 0 | |
| | P1 | 70 | 11 | −38.06925615 | 0 | |
| | P3 | 70 | 15 | −52.93814738 | 0 | |
| A9 (third time) | P8 | 70 | 0 | 11.09493779 | 11.09493779 | * |
| | P14 | 70 | 3 | −4.002206259 | 0 | |
| | P13 | 70 | 3 | −7.423207529 | 0 | |
| | P9 | 70 | 4 | −12.86862853 | 0 | |
| | P11 | 70 | 5 | −16.33875378 | 0 | |
| | P5 | 70 | 5 | −16.47391643 | 0 | |
| | P7 | 70 | 6 | −16.69783305 | 0 | |
| | P4 | 70 | 6 | −19.14631397 | 0 | |
| | P6 | 70 | 6 | −20.14117199 | 0 | |
| | P12 | 70 | 7 | −26.15151281 | 0 | |
| | P1 | 70 | 9 | −26.99723958 | 0 | |
| | P10 | 70 | 8 | −27.98366961 | 0 | |
| | P3 | 70 | 9 | −28.31509725 | 0 | |
| | P2 | 70 | 9 | −30.35807931 | 0 | |

TABLE 13
Verification tests for paternity testing

| | Candidate male | Number of | Number of | | | Confidence |
|---|---|---|---|---|---|---|
| Offspring SN | parent SN | typing sites | mismatched sites | LOD value | Δ | coefficient |
| A10 (first time) | P9 | 70 | 1 | 13.79585617 | 13.79585617 | * |
| | P14 | 70 | 5 | −8.717864064 | 0 | |
| | P7 | 70 | 5 | −10.5135352 | 0 | |
| | P8 | 70 | 5 | −14.36603191 | 0 | |
| | P11 | 70 | 8 | −21.77191201 | 0 | |
| | P13 | 70 | 8 | −26.36108471 | 0 | |
| | P3 | 70 | 9 | −29.62516786 | 0 | |
| | P1 | 70 | 10 | −30.90277195 | 0 | |
| | P4 | 70 | 10 | −35.00326985 | 0 | |
| | P10 | 70 | 10 | −36.09838327 | 0 | |
| | P6 | 70 | 11 | −37.66695494 | 0 | |
| | P12 | 70 | 11 | −38.48845812 | 0 | |
| | P5 | 70 | 11 | −41.25969161 | 0 | |
| | P2 | 70 | 12 | −43.02388195 | 0 | |
| A10 (second time) | P9 | 70 | 1 | 8.701582645 | 8.701582645 | * |
| | P11 | 70 | 4 | −7.975589439 | 0 | |
| | P4 | 70 | 5 | −13.30957167 | 0 | |
| | P5 | 70 | 5 | −16.92731292 | 0 | |
| | P8 | 70 | 7 | −20.29594817 | 0 | |
| | P6 | 70 | 7 | −20.64772855 | 0 | |
| | P14 | 70 | 7 | −22.70047987 | 0 | |
| | P7 | 70 | 8 | −23.73269357 | 0 | |
| | P10 | 70 | 8 | −25.24322179 | 0 | |
| | P2 | 70 | 9 | −31.92767407 | 0 | |
| | P1 | 70 | 10 | −33.04279883 | 0 | |
| | P13 | 70 | 10 | −33.57778844 | 0 | |
| | P12 | 70 | 11 | −38.0287652 | 0 | |
| | P3 | 70 | 12 | −42.89185259 | 0 | |
| A10 (third time) | P9 | 70 | 0 | 9.515947688 | 9.515947688 | * |
| | P13 | 70 | 3 | −8.686951703 | 0 | |
| | P7 | 70 | 5 | −9.594593301 | 0 | |
| | P4 | 70 | 4 | −11.53505627 | 0 | |
| | P12 | 70 | 5 | −12.06219714 | 0 | |
| | P14 | 70 | 4 | −12.70204571 | 0 | |
| | P11 | 70 | 5 | −13.37686993 | 0 | |
| | P8 | 70 | 5 | −14.35488138 | 0 | |
| | P5 | 70 | 5 | −17.38586597 | 0 | |
| | P10 | 70 | 7 | −25.41374906 | 0 | |
| | P1 | 70 | 8 | −25.54819939 | 0 | |
| | P2 | 70 | 8 | −27.79917 | 0 | |
| | P3 | 70 | 9 | −32.02940943 | 0 | |
| | P6 | 70 | 9 | −34.30218495 | 0 | |

Although the above example has described the present disclosure in detail, it is only apart of, not all of, the examples of the present disclosure. Other examples may also be obtained by persons based on the example without creative efforts, and all of these examples shall fall within the protection scope of the present disclosure.

1. A single nucleotide polymorphism (SNP) molecular marker combination for genetic relationship identification of a Huaxi cattle, comprising at least 70 of SNP molecular markers as follows:

| | | | | Minimum |
|---|---|---|---|---|
| | | | Base | allele |
| | Chro- | | vari- | frequency |
| SNP name | mosome | Position | ation | (MAF) |
| chr1: 146736 | 1 | 146736 | C/T | 0.4996 |
| BovineHD0100001067 | 1 | 3232948 | G/A | 0.4992 |
| BovineHD0100001535 | 1 | 4740225 | C/A | 0.4976 |
| BovineHD0100002084 | 1 | 6397764 | G/A | 0.4984 |
| chr1: 8222595 | 1 | 8222595 | C/T | 0.4976 |
| chr1: 10268092 | 1 | 10268092 | T/G | 0.4669 |
| chr1: 12788006 | 1 | 12788006 | C/T | 0.488 |
| chr1: 14370430 | 1 | 14370430 | C/T | 0.4677 |
| chr1: 17929625 | 1 | 17929625 | A/C | 0.496 |
| Hapmap51222-BTA-39456 | 1 | 19805218 | T/C | 0.4141 |
| chr1: 20746789 | 1 | 20746789 | T/C | 0.494 |
| chr1: 22937855 | 1 | 22937855 | G/A | 0.4928 |
| chr1: 24948004 | 1 | 24948004 | G/A | 0.4952 |
| chr1: 27982761 | 1 | 27982761 | T/C | 0.4509 |
| BovineHD0100008442 | 1 | 28450059 | G/A | 0.4101 |
| chr1: 30343940 | 1 | 30343940 | C/T | 0.4776 |
| chr1: 31151169 | 1 | 31151169 | T/C | 0.4976 |
| BovineHD0100009944 | 1 | 34665393 | A/G | 0.498 |
| chr1: 36240622 | 1 | 36240622 | T/C | 0.4149 |
| chr1: 38821576 | 1 | 38821576 | G/C | 0.4992 |
| chr1: 41190611 | 1 | 41190611 | A/G | 0.4952 |
| chr1: 43473529 | 1 | 43473529 | C/G | 0.4984 |
| chr1: 46242711 | 1 | 46242711 | A/G | 0.4992 |
| BovineHD0100013967 | 1 | 49559687 | T/G | 0.5 |
| chr1: 51894494 | 1 | 51894494 | C/A | 0.4956 |
| chr1: 53589018 | 1 | 53589018 | A/G | 0.4992 |
| BovineHD0100015794 | 1 | 56127149 | A/G | 0.4908 |
| BovineHD0100016547 | 1 | 58465963 | G/A | 0.4992 |
| Hapmap48290-BTA-31675 | 1 | 61069157 | A/G | 0.4968 |
| chr1: 63123889 | 1 | 63123889 | C/T | 0.4876 |
| chr1: 65202434 | 1 | 65202434 | C/T | 0.4764 |
| chr1: 67249619 | 1 | 67249619 | C/T | 0.4944 |
| chr1: 69353459 | 1 | 69353459 | T/C | 0.4944 |
| chr1: 71116985 | 1 | 71116985 | G/A | 0.474 |
| chr1: 73343620 | 1 | 73343620 | T/C | 0.4976 |
| chr1: 75567962 | 1 | 75567962 | C/T | 0.3954 |
| chr1: 77468186 | 1 | 77468186 | C/T | 0.5 |
| chr1: 79185721 | 1 | 79185721 | T/C | 0.4828 |
| chr1: 81072008 | 1 | 81072008 | T/C | 0.4117 |
| chr1: 83093568 | 1 | 83093568 | T/C | 0.4976 |
| chr1: 85247068 | 1 | 85247068 | C/A | 0.3674 |
| chr1: 87604486 | 1 | 87604486 | A/G | 0.4976 |
| BovineHD0100025718 | 1 | 90391338 | T/G | 0.4617 |
| chr1: 92106250 | 1 | 92106250 | C/T | 0.4996 |
| chr1: 94141904 | 1 | 94141904 | T/C | 0.4852 |
| chr1: 96618535 | 1 | 96618535 | A/T | 0.4992 |
| chr1: 98213895 | 1 | 98213895 | G/A | 0.4976 |
| chr1: 102597974 | 1 | 102597974 | A/T | 0.4792 |
| chr1: 104553440 | 1 | 104553440 | C/A | 0.5 |
| chr1: 106360058 | 1 | 106360058 | A/G | 0.4948 |
| chr1: 108109512 | 1 | 108109512 | A/G | 0.5 |
| chr1: 111219204 | 1 | 111219204 | G/T | 0.4968 |
| chr1: 113316161 | 1 | 113316161 | A/G | 0.4996 |
| chr1: 115307210 | 1 | 115307210 | C/T | 0.4996 |
| chr1: 117232512 | 1 | 117232512 | G/A | 0.4988 |
| chr1: 119142301 | 1 | 119142301 | A/G | 0.4996 |
| chr1: 121619169 | 1 | 121619169 | C/G | 0.498 |
| chr1: 123306221 | 1 | 123306221 | T/C | 0.4569 |
| chr1: 125963762 | 1 | 125963762 | T/C | 0.4992 |
| chr1: 127473224 | 1 | 127473224 | C/T | 0.3774 |
| chr1: 129312196 | 1 | 129312196 | T/C | 0.4872 |
| chr1: 131338591 | 1 | 131338591 | C/T | 0.4936 |
| chr1: 133231492 | 1 | 133231492 | C/T | 0.4613 |
| chr1: 135638938 | 1 | 135638938 | G/A | 0.4984 |
| chr1: 137647060 | 1 | 137647060 | G/T | 0.4976 |
| chr1: 139172793 | 1 | 139172793 | T/G | 0.4956 |
| chr1: 141796859 | 1 | 141796859 | C/G | 0.4537 |
| BovineHD0100041816 | 1 | 144840991 | G/A | 0.4988 |
| chr1: 147365032 | 1 | 147365032 | A/G | 0.492 |
| chr1: 149696295 | 1 | 149696295 | G/T | 0.4768 |
| chr1: 151931941 | 1 | 151931941 | C/T | 0.4968 |
| chr1: 153033656 | 1 | 153033656 | C/T | 0.4868 |
| chr1: 157013585 | 1 | 157013585 | A/G | 0.494 |
| chr2: 2292914 | 2 | 2292914 | C/T | 0.498 |
| chr2: 3201445 | 2 | 3201445 | T/G | 0.4481 |
| chr2: 6547325 | 2 | 6547325 | C/G | 0.4924 |
| BovineHD0200002751 | 2 | 9691620 | C/T | 0.4629 |
| chr2: 10515607 | 2 | 10515607 | G/A | 0.4984 |
| BovineHD0200003077 | 2 | 11166355 | T/G | 0.4812 |
| chr2: 12469133 | 2 | 12469133 | A/G | 0.4924 |
| chr2: 14337459 | 2 | 14337459 | C/A | 0.4988 |
| chr2: 16361378 | 2 | 16361378 | T/C | 0.4752 |
| chr2: 18736141 | 2 | 18736141 | G/A | 0.4832 |
| chr2: 20899037 | 2 | 20899037 | G/A | 0.4956 |
| chr2: 23048877 | 2 | 23048877 | A/C | 0.4992 |
| chr2: 25766917 | 2 | 25766917 | A/G | 0.4069 |
| chr2: 27704806 | 2 | 27704806 | C/A | 0.4896 |
| chr2: 29923519 | 2 | 29923519 | C/A | 0.4653 |
| chr2: 31692834 | 2 | 31692834 | T/G | 0.4992 |
| chr2: 33882604 | 2 | 33882604 | G/A | 0.4153 |
| chr2: 35706579 | 2 | 35706579 | C/G | 0.4884 |
| chr2: 38317092 | 2 | 38317092 | G/A | 0.494 |
| chr2: 40402873 | 2 | 40402873 | G/A | 0.4988 |
| chr2: 42785850 | 2 | 42785850 | A/C | 0.4832 |
| chr2: 45298638 | 2 | 45298638 | T/C | 0.4952 |
| chr2: 47490334 | 2 | 47490334 | C/G | 0.4924 |
| chr2: 50246865 | 2 | 50246865 | A/T | 0.4996 |
| chr2: 53125067 | 2 | 53125067 | T/G | 0.4704 |
| chr2: 55611799 | 2 | 55611799 | A/T | 0.4972 |
| chr2: 57700577 | 2 | 57700577 | A/C | 0.4904 |
| BovineHD0200018019 | 2 | 62411616 | C/T | 0.4964 |
| chr2: 63275266 | 2 | 63275266 | T/A | 0.4988 |
| BovineHD0200018552 | 2 | 64242565 | A/G | 0.478 |
| chr2: 68158765 | 2 | 68158765 | G/C | 0.4912 |
| chr2: 70740356 | 2 | 70740356 | C/G | 0.4984 |
| chr2: 73566014 | 2 | 73566014 | T/C | 0.4421 |
| chr2: 75948236 | 2 | 75948236 | G/A | 0.4968 |
| BovineHD0200022742 | 2 | 79151817 | G/A | 0.4996 |
| chr2: 81673931 | 2 | 81673931 | G/A | 0.3966 |
| chr2: 83323019 | 2 | 83323019 | T/C | 0.4421 |
| chr2: 86824344 | 2 | 86824344 | G/A | 0.4964 |
| chr2: 89335404 | 2 | 89335404 | T/C | 0.5 |
| chr2: 91648591 | 2 | 91648591 | G/A | 0.498 |
| BovineHD0200027563 | 2 | 95935954 | T/C | 0.498 |
| chr2: 97928774 | 2 | 97928774 | T/C | 0.4928 |
| chr2: 99749502 | 2 | 99749502 | C/T | 0.4261 |
| chr2: 102483918 | 2 | 102483918 | C/G | 0.4022 |
| BovineHD0200030211 | 2 | 105005873 | C/T | 0.4844 |
| chr2: 107473778 | 2 | 107473778 | G/A | 0.4724 |
| chr2: 109312516 | 2 | 109312516 | T/C | 0.4936 |
| chr2: 111984199 | 2 | 111984199 | A/G | 0.4844 |
| chr2: 114011690 | 2 | 114011690 | T/G | 0.4996 |
| chr2: 116006681 | 2 | 116006681 | C/G | 0.4896 |
| chr2: 118519219 | 2 | 118519219 | C/T | 0.3522 |
| chr2: 121231584 | 2 | 121231584 | C/G | 0.4453 |
| chr2: 123977838 | 2 | 123977838 | A/G | 0.4784 |
| chr2: 125154883 | 2 | 125154883 | T/C | 0.4972 |
| chr2: 127550880 | 2 | 127550880 | C/T | 0.4157 |
| chr2: 129689230 | 2 | 129689230 | C/A | 0.4992 |
| chr2: 131912580 | 2 | 131912580 | G/A | 0.5 |
| chr2: 133126495 | 2 | 133126495 | A/G | 0.4844 |
| chr2: 135007150 | 2 | 135007150 | A/C | 0.47 |
| chr2: 136693511 | 2 | 136693511 | A/T | 0.4988 |
| BovineHD0300000204 | 3 | 922000 | A/C | 0.4892 |
| BovineHD0300000351 | 3 | 1296645 | T/C | 0.4996 |
| BovineHD0300000547 | 3 | 2038037 | C/T | 0.4796 |
| chr3: 2972559 | 3 | 2972559 | G/A | 0.4856 |
| chr3: 4609284 | 3 | 4609284 | T/A | 0.4205 |
| chr3: 6714473 | 3 | 6714473 | G/A | 0.4241 |
| chr3: 8163083 | 3 | 8163083 | C/A | 0.4988 |
| chr3: 10889683 | 3 | 10889683 | T/C | 0.4996 |
| chr3: 12700815 | 3 | 12700815 | T/G | 0.4992 |
| chr3: 14888178 | 3 | 14888178 | A/T | 0.4685 |
| chr3: 16774367 | 3 | 16774367 | C/G | 0.4988 |
| chr3: 18214907 | 3 | 18214907 | G/A | 0.4856 |
| chr3: 20227589 | 3 | 20227589 | A/T | 0.4956 |
| chr3: 22888010 | 3 | 22888010 | T/C | 0.4908 |
| chr3: 24121607 | 3 | 24121607 | G/A | 0.4916 |
| chr3: 26062421 | 3 | 26062421 | A/C | 0.5 |
| chr3: 28243923 | 3 | 28243923 | T/A | 0.4988 |
| chr3: 31210779 | 3 | 31210779 | C/T | 0.4996 |
| chr3: 33410492 | 3 | 33410492 | A/C | 0.4976 |
| chr3: 35703444 | 3 | 35703444 | A/C | 0.4912 |
| chr3: 37862314 | 3 | 37862314 | T/C | 0.4848 |
| chr3: 40047483 | 3 | 40047483 | C/A | 0.4976 |
| chr3: 42347013 | 3 | 42347013 | A/G | 0.4992 |
| chr3: 46141906 | 3 | 46141906 | T/C | 0.49 |
| chr3: 48531987 | 3 | 48531987 | C/T | 0.4888 |
| chr3: 50087938 | 3 | 50087938 | C/A | 0.4908 |
| chr3: 52363329 | 3 | 52363329 | G/C | 0.3558 |
| chr3: 55465323 | 3 | 55465323 | A/G | 0.4896 |
| chr3: 58278551 | 3 | 58278551 | T/C | 0.4948 |
| chr3: 60872835 | 3 | 60872835 | C/T | 0.4932 |
| chr3: 63972726 | 3 | 63972726 | A/G | 0.4776 |
| chr3: 65931583 | 3 | 65931583 | G/A | 0.4984 |
| chr3: 68033961 | 3 | 68033961 | G/A | 0.496 |
| chr3: 71381417 | 3 | 71381417 | T/C | 0.4748 |
| chr3: 73731721 | 3 | 73731721 | T/C | 0.4149 |
| chr3: 75720895 | 3 | 75720895 | T/A | 0.492 |
| chr3: 78450019 | 3 | 78450019 | C/A | 0.4936 |
| chr3: 80270744 | 3 | 80270744 | C/T | 0.4377 |
| chr3: 83162493 | 3 | 83162493 | T/C | 0.4992 |
| chr3: 85988901 | 3 | 85988901 | T/C | 0.4892 |
| chr3: 87656169 | 3 | 87656169 | G/A | 0.4976 |
| chr3: 89847174 | 3 | 89847174 | G/T | 0.4984 |
| chr3: 91558792 | 3 | 91558792 | C/A | 0.4968 |
| chr3: 93499656 | 3 | 93499656 | T/C | 0.498 |
| chr3: 95012162 | 3 | 95012162 | A/T | 0.498 |
| chr3: 97148386 | 3 | 97148386 | T/G | 0.4984 |
| chr3: 100912209 | 3 | 100912209 | T/C | 0.4281 |
| chr3: 102515238 | 3 | 102515238 | A/G | 0.5 |
| chr3: 104164934 | 3 | 104164934 | C/T | 0.4964 |
| chr3: 106219168 | 3 | 106219168 | G/C | 0.4996 |
| chr3: 108180618 | 3 | 108180618 | G/C | 0.4728 |
| BovineHD4100002462 | 3 | 110010745 | T/C | 0.4976 |
| chr3: 112430477 | 3 | 112430477 | C/T | 0.4992 |
| chr3: 114619239 | 3 | 114619239 | G/A | 0.4916 |
| chr3: 116452622 | 3 | 116452622 | G/C | 0.4824 |
| chr3: 118148477 | 3 | 118148477 | A/G | 0.5 |
| chr3: 119657526 | 3 | 119657526 | C/T | 0.4864 |
| chr3: 121175246 | 3 | 121175246 | T/C | 0.4984 |
| chr4: 3861427 | 4 | 3861427 | T/C | 0.4988 |
| BovineHD4100002612 | 4 | 5478011 | A/G | 0.4924 |
| BovineHD0400001913 | 4 | 6549577 | A/G | 0.472 |
| BovineHD0400002141 | 4 | 7138504 | T/C | 0.498 |
| chr4: 9021510 | 4 | 9021510 | C/T | 0.4461 |
| chr4: 11577418 | 4 | 11577418 | T/C | 0.4904 |
| chr4: 13098564 | 4 | 13098564 | G/T | 0.4956 |
| chr4: 15359883 | 4 | 15359883 | C/T | 0.3806 |
| chr4: 17779954 | 4 | 17779954 | C/T | 0.4992 |
| chr4: 19078872 | 4 | 19078872 | T/C | 0.4992 |
| chr4: 22645768 | 4 | 22645768 | G/A | 0.4976 |
| chr4: 24307253 | 4 | 24307253 | G/C | 0.403 |
| chr4: 26020550 | 4 | 26020550 | G/C | 0.4992 |
| chr4: 28165801 | 4 | 28165801 | T/G | 0.4269 |
| chr4: 30471896 | 4 | 30471896 | C/A | 0.4944 |
| chr4: 32417299 | 4 | 32417299 | C/A | 0.4984 |
| chr4: 35167308 | 4 | 35167308 | G/T | 0.4996 |
| chr4: 37653081 | 4 | 37653081 | C/T | 0.4996 |
| chr4: 41047723 | 4 | 41047723 | G/A | 0.4784 |
| chr4: 44408279 | 4 | 44408279 | C/A | 0.4956 |
| chr4: 48947495 | 4 | 48947495 | C/A | 0.4996 |
| chr4: 51736700 | 4 | 51736700 | T/C | 0.4984 |
| chr4: 53557315 | 4 | 53557315 | G/A | 0.4517 |
| chr4: 56251739 | 4 | 56251739 | A/T | 0.4617 |
| chr4: 59377877 | 4 | 59377877 | T/A | 0.498 |
| chr4: 62116676 | 4 | 62116676 | T/C | 0.494 |
| chr4: 64220533 | 4 | 64220533 | A/G | 0.4964 |
| chr4: 66072087 | 4 | 66072087 | C/T | 0.4429 |
| chr4: 68072014 | 4 | 68072014 | A/C | 0.4764 |
| chr4: 70958591 | 4 | 70958591 | G/T | 0.4461 |
| chr4: 72419909 | 4 | 72419909 | A/G | 0.4972 |
| chr4: 74319680 | 4 | 74319680 | C/T | 0.4856 |
| chr4: 76069001 | 4 | 76069001 | G/A | 0.4932 |
| chr4: 78733527 | 4 | 78733527 | A/G | 0.4996 |
| chr4: 80412321 | 4 | 80412321 | G/A | 0.4876 |
| chr4: 82284742 | 4 | 82284742 | C/T | 0.4557 |
| chr4: 84205752 | 4 | 84205752 | G/C | 0.4613 |
| chr4: 86290264 | 4 | 86290264 | G/A | 0.4936 |
| chr4: 88179396 | 4 | 88179396 | G/A | 0.4984 |
| chr4: 90103827 | 4 | 90103827 | A/G | 0.4936 |
| chr4: 92469122 | 4 | 92469122 | G/T | 0.4996 |
| chr4: 94734183 | 4 | 94734183 | A/C | 0.4213 |
| chr4: 96100801 | 4 | 96100801 | T/C | 0.4984 |
| chr4: 98539972 | 4 | 98539972 | G/C | 0.4984 |
| chr4: 100397123 | 4 | 100397123 | C/G | 0.4964 |
| chr4: 102560075 | 4 | 102560075 | G/T | 0.4956 |
| chr4: 104245564 | 4 | 104245564 | G/A | 0.4924 |
| chr4: 106099249 | 4 | 106099249 | A/T | 0.494 |
| chr4: 108302747 | 4 | 108302747 | G/C | 0.4996 |
| chr4: 110365140 | 4 | 110365140 | G/A | 0.4988 |
| chr4: 112014359 | 4 | 112014359 | T/C | 0.5 |
| chr4: 114636283 | 4 | 114636283 | C/T | 0.4844 |
| chr4: 116226917 | 4 | 116226917 | C/T | 0.4988 |
| chr4: 119239489 | 4 | 119239489 | T/A | 0.4996 |
| BovineHD0500000007 | 5 | 31485 | C/T | 0.4637 |
| BovineHD0500000460 | 5 | 1797290 | G/T | 0.4992 |
| chr5: 2610987 | 5 | 2610987 | G/T | 0.49 |
| BovineHD0500000773 | 5 | 3012267 | A/G | 0.49 |
| chr5: 5068243 | 5 | 5068243 | G/C | 0.4928 |
| chr5: 6425820 | 5 | 6425820 | T/C | 0.4848 |
| chr5: 8523790 | 5 | 8523790 | A/C | 0.4728 |
| chr5: 9903460 | 5 | 9903460 | A/G | 0.482 |
| chr5: 11064016 | 5 | 11064016 | A/G | 0.4976 |
| chr5: 13468378 | 5 | 13468378 | T/C | 0.4968 |
| chr5: 15650751 | 5 | 15650751 | G/A | 0.5 |
| chr5: 17031046 | 5 | 17031046 | G/T | 0.4868 |
| chr5: 19601695 | 5 | 19601695 | G/A | 0.4665 |
| chr5: 22283765 | 5 | 22283765 | A/C | 0.4952 |
| chr5: 25309909 | 5 | 25309909 | T/G | 0.4884 |
| chr5: 27998047 | 5 | 27998047 | T/G | 0.4613 |
| chr5: 29249825 | 5 | 29249825 | C/T | 0.498 |
| chr5: 32734860 | 5 | 32734860 | G/A | 0.395 |
| chr5: 34934298 | 5 | 34934298 | G/A | 0.4824 |
| chr5: 36100135 | 5 | 36100135 | A/G | 0.4984 |
| chr5: 38860266 | 5 | 38860266 | C/T | 0.4952 |
| chr5: 40539755 | 5 | 40539755 | C/T | 0.4992 |
| chr5: 42546535 | 5 | 42546535 | C/A | 0.5 |
| chr5: 45596322 | 5 | 45596322 | T/C | 0.4756 |
| chr5: 48383398 | 5 | 48383398 | T/A | 0.4984 |
| chr5: 50356341 | 5 | 50356341 | C/T | 0.4992 |
| chr5: 52837788 | 5 | 52837788 | A/G | 0.4984 |
| chr5: 55094769 | 5 | 55094769 | C/A | 0.4996 |
| chr5: 57563283 | 5 | 57563283 | C/T | 0.4992 |
| chr5: 61938027 | 5 | 61938027 | T/C | 0.4988 |
| chr5: 63674646 | 5 | 63674646 | T/C | 0.5 |
| chr5: 65117330 | 5 | 65117330 | T/A | 0.5 |
| chr5: 69355384 | 5 | 69355384 | C/A | 0.498 |
| chr5: 71878536 | 5 | 71878536 | G/A | 0.5 |
| chr5: 74763956 | 5 | 74763956 | C/T | 0.4972 |
| chr5: 75632140 | 5 | 75632140 | C/T | 0.4916 |
| chr5: 78472118 | 5 | 78472118 | C/T | 0.4736 |
| chr5: 80990355 | 5 | 80990355 | G/A | 0.4996 |
| BovineHD0500023615 | 5 | 83434257 | T/C | 0.496 |
| chr5: 85549231 | 5 | 85549231 | G/A | 0.403 |
| chr5: 87397204 | 5 | 87397204 | G/C | 0.4864 |
| chr5: 90018451 | 5 | 90018451 | G/C | 0.4944 |
| chr5: 92365101 | 5 | 92365101 | C/T | 0.4673 |
| chr5: 94219588 | 5 | 94219588 | T/C | 0.4948 |
| chr5: 96207260 | 5 | 96207260 | T/C | 0.4984 |
| chr5: 98156010 | 5 | 98156010 | T/C | 0.4996 |
| chr5: 100451709 | 5 | 100451709 | G/A | 0.5 |
| chr5: 103778185 | 5 | 103778185 | C/A | 0.4952 |
| BovineHD0500030582 | 5 | 106568560 | T/C | 0.472 |
| chr5: 108509149 | 5 | 108509149 | G/A | 0.4621 |
| chr5: 110026273 | 5 | 110026273 | A/G | 0.492 |
| chr5: 112310553 | 5 | 112310553 | C/T | 0.4784 |
| chr5: 114677995 | 5 | 114677995 | A/G | 0.496 |
| chr5: 116585246 | 5 | 116585246 | G/A | 0.4736 |
| chr5: 118068937 | 5 | 118068937 | G/A | 0.4996 |
| chr5: 120597412 | 5 | 120597412 | A/G | 0.4669 |
| BovineHD0600000946 | 6 | 3641139 | A/G | 0.4976 |
| BovineHD0600000994 | 6 | 3807975 | G/A | 0.4996 |
| BovineHD0600001372 | 6 | 5230318 | G/A | 0.3502 |
| BovineHD0600001622 | 6 | 7436488 | A/G | 0.4864 |
| chr6: 8715560 | 6 | 8715560 | T/C | 0.4453 |
| chr6: 10275743 | 6 | 10275743 | T/A | 0.486 |
| chr6: 12025526 | 6 | 12025526 | C/A | 0.4996 |
| chr6: 13851956 | 6 | 13851956 | T/C | 0.4932 |
| chr6: 15923688 | 6 | 15923688 | T/G | 0.4968 |
| chr6: 18191288 | 6 | 18191288 | A/G | 0.4944 |
| chr6: 20029937 | 6 | 20029937 | T/G | 0.49 |
| chr6: 22018522 | 6 | 22018522 | T/G | 0.4964 |
| chr6: 24308611 | 6 | 24308611 | T/C | 0.4389 |
| chr6: 25955274 | 6 | 25955274 | G/A | 0.4868 |
| chr6: 27127135 | 6 | 27127135 | G/A | 0.4984 |
| chr6: 30344055 | 6 | 30344055 | C/T | 0.4868 |
| chr6: 32995850 | 6 | 32995850 | T/C | 0.4988 |
| chr6: 36295718 | 6 | 36295718 | G/T | 0.4776 |
| chr6: 39465845 | 6 | 39465845 | T/C | 0.498 |
| chr6: 42163850 | 6 | 42163850 | G/A | 0.5 |
| chr6: 45430248 | 6 | 45430248 | A/G | 0.4948 |
| chr6: 48405707 | 6 | 48405707 | T/A | 0.4273 |
| chr6: 50368143 | 6 | 50368143 | T/A | 0.4904 |
| chr6: 52091508 | 6 | 52091508 | C/G | 0.4892 |
| chr6: 54763160 | 6 | 54763160 | C/G | 0.4996 |
| chr6: 56962144 | 6 | 56962144 | T/C | 0.4968 |
| chr6: 58716750 | 6 | 58716750 | T/C | 0.4948 |
| chr6: 60131226 | 6 | 60131226 | G/A | 0.496 |
| chr6: 62381859 | 6 | 62381859 | T/C | 0.4996 |
| chr6: 65818645 | 6 | 65818645 | A/G | 0.4996 |
| chr6: 68556443 | 6 | 68556443 | T/C | 0.4984 |
| chr6: 72859610 | 6 | 72859610 | A/G | 0.4972 |
| chr6: 75090494 | 6 | 75090494 | T/C | 0.5 |
| chr6: 87995541 | 6 | 87995541 | G/T | 0.4956 |
| chr6: 97061781 | 6 | 97061781 | G/A | 0.4692 |
| chr6: 108816557 | 6 | 108816557 | C/T | 0.4792 |
| chr6: 117500189 | 6 | 117500189 | T/C | 0.4645 |
| chr7: 323290 | 7 | 323290 | G/A | 0.4437 |
| chr7: 2636459 | 7 | 2636459 | T/C | 0.5 |
| BovineHD0700000816 | 7 | 2920267 | A/G | 0.4992 |
| BovineHD0700000906 | 7 | 3330103 | T/C | 0.4796 |
| chr7: 5497295 | 7 | 5497295 | C/T | 0.4996 |
| chr7: 7969340 | 7 | 7969340 | G/T | 0.4956 |
| chr7: 10003983 | 7 | 10003983 | T/C | 0.4992 |
| chr7: 13202771 | 7 | 13202771 | A/G | 0.4984 |
| chr7: 16922116 | 7 | 16922116 | T/C | 0.4992 |
| chr7: 18729157 | 7 | 18729157 | G/A | 0.4657 |
| chr7: 20314935 | 7 | 20314935 | T/C | 0.4988 |
| chr7: 22363112 | 7 | 22363112 | C/T | 0.4437 |
| chr7: 24361453 | 7 | 24361453 | C/T | 0.4956 |
| BovineHD0700007619 | 7 | 27360142 | G/A | 0.4952 |
| chr7: 29102061 | 7 | 29102061 | A/G | 0.5 |
| chr7: 31395937 | 7 | 31395937 | G/A | 0.4992 |
| chr7: 33488283 | 7 | 33488283 | T/C | 0.4984 |
| chr7: 35127491 | 7 | 35127491 | T/C | 0.4996 |
| chr7: 37079045 | 7 | 37079045 | T/G | 0.4988 |
| BovineHD0700011845 | 7 | 40874649 | A/G | 0.4876 |
| chr7: 42921853 | 7 | 42921853 | T/C | 0.4593 |
| chr7: 46690297 | 7 | 46690297 | G/A | 0.4844 |
| chr7: 48884947 | 7 | 48884947 | A/C | 0.4972 |
| chr7: 50250949 | 7 | 50250949 | G/A | 0.4948 |
| chr7: 53296158 | 7 | 53296158 | G/A | 0.498 |
| chr7: 55798969 | 7 | 55798969 | C/A | 0.4988 |
| chr7: 58187926 | 7 | 58187926 | C/T | 0.4956 |
| chr7: 61088591 | 7 | 61088591 | T/A | 0.4712 |
| chr7: 65393898 | 7 | 65393898 | C/G | 0.494 |
| chr7: 67286820 | 7 | 67286820 | C/G | 0.4844 |
| chr7: 69280676 | 7 | 69280676 | G/A | 0.4972 |
| chr7: 71538319 | 7 | 71538319 | G/A | 0.4972 |
| chr7: 73794744 | 7 | 73794744 | T/C | 0.498 |
| chr7: 75140866 | 7 | 75140866 | G/C | 0.4992 |
| chr7: 77176754 | 7 | 77176754 | C/T | 0.4824 |
| chr7: 79798840 | 7 | 79798840 | C/G | 0.4848 |
| BovineHD0700023890 | 7 | 82015442 | A/C | 0.4988 |
| chr7: 94484358 | 7 | 94484358 | G/A | 0.4888 |
| chr7: 103520087 | 7 | 103520087 | C/T | 0.4964 |
| chr7: 109212612 | 7 | 109212612 | A/C | 0.5 |
| chr8: 52977 | 8 | 52977 | C/T | 0.4996 |
| ARS-USMARC-Parent- | 8 | 1554706 | G/A | 0.4964 |
| DQ916058-rs29016146 | | | | |
| Hapmap46330-BTA-95948 | 8 | 3533444 | T/A | 0.4964 |
| UA-IFASA-5111 | 8 | 3922416 | A/G | 0.4868 |
| chr8: 6024854 | 8 | 6024854 | A/G | 0.4944 |
| chr8: 9054284 | 8 | 9054284 | A/T | 0.3834 |
| chr8: 11592103 | 8 | 11592103 | G/A | 0.4916 |
| chr8: 13246773 | 8 | 13246773 | T/G | 0.4964 |
| chr8: 15507208 | 8 | 15507208 | G/T | 0.4996 |
| chr8: 17122844 | 8 | 17122844 | A/G | 0.4988 |
| chr8: 19494843 | 8 | 19494843 | A/T | 0.4856 |
| chr8: 21255732 | 8 | 21255732 | A/G | 0.4984 |
| chr8: 24452617 | 8 | 24452617 | A/T | 0.4912 |
| BovineHD0800007910 | 8 | 26150623 | A/G | 0.4776 |
| chr8: 29128317 | 8 | 29128317 | T/A | 0.4964 |
| chr8: 31213020 | 8 | 31213020 | C/G | 0.4976 |
| chr8: 32901857 | 8 | 32901857 | A/G | 0.4968 |
| chr8: 34910132 | 8 | 34910132 | T/C | 0.4992 |
| chr8: 37544170 | 8 | 37544170 | G/C | 0.4992 |
| chr8: 39903026 | 8 | 39903026 | T/A | 0.3966 |
| chr8: 41771334 | 8 | 41771334 | A/G | 0.4996 |
| chr8: 42954715 | 8 | 42954715 | C/G | 0.4984 |
| chr8: 45323917 | 8 | 45323917 | A/T | 0.4984 |
| chr8: 47935064 | 8 | 47935064 | A/G | 0.4828 |
| chr8: 50505013 | 8 | 50505013 | A/G | 0.4992 |
| chr8: 52181043 | 8 | 52181043 | A/T | 0.4876 |
| chr8: 54046042 | 8 | 54046042 | C/G | 0.4968 |
| chr8: 56303275 | 8 | 56303275 | C/T | 0.4988 |
| chr8: 58472144 | 8 | 58472144 | T/C | 0.4736 |
| chr8: 60302978 | 8 | 60302978 | C/A | 0.4848 |
| BovineHD0800018780 | 8 | 62878834 | A/G | 0.4996 |
| chr8: 64364896 | 8 | 64364896 | A/G | 0.4924 |
| chr8: 66116974 | 8 | 66116974 | A/G | 0.4916 |
| chr8: 68298246 | 8 | 68298246 | G/A | 0.4864 |
| chr8: 70059755 | 8 | 70059755 | C/T | 0.4824 |
| chr8: 72761382 | 8 | 72761382 | C/A | 0.4988 |
| chr8: 74068851 | 8 | 74068851 | A/G | 0.4984 |
| chr8: 77187323 | 8 | 77187323 | C/T | 0.4916 |
| chr8: 79249577 | 8 | 79249577 | A/T | 0.4952 |
| chr8: 81936011 | 8 | 81936011 | C/T | 0.4976 |
| chr8: 84900873 | 8 | 84900873 | C/T | 0.496 |
| BovineHD0800026578 | 8 | 89625431 | C/T | 0.498 |
| chr8: 91908606 | 8 | 91908606 | G/T | 0.4888 |
| BovineHD0800028888 | 8 | 97833606 | C/T | 0.4952 |
| chr8: 106296663 | 8 | 106296663 | G/A | 0.4996 |
| chr9: 118327 | 9 | 118327 | A/T | 0.486 |
| chr9: 4553903 | 9 | 4553903 | T/C | 0.494 |
| BovineHD0900001546 | 9 | 7173058 | C/A | 0.4992 |
| BovineHD0900002201 | 9 | 9060667 | A/C | 0.4996 |
| BovineHD0900002717 | 9 | 10949242 | A/G | 0.4968 |
| chr9: 11988685 | 9 | 11988685 | A/G | 0.4964 |
| chr9: 14102277 | 9 | 14102277 | C/A | 0.4645 |
| chr9: 16050438 | 9 | 16050438 | T/C | 0.4976 |
| chr9: 18927655 | 9 | 18927655 | T/C | 0.4968 |
| chr9: 20941076 | 9 | 20941076 | T/C | 0.4896 |
| chr9: 23183945 | 9 | 23183945 | T/G | 0.4876 |
| chr9: 25342529 | 9 | 25342529 | G/T | 0.4916 |
| chr9: 27435788 | 9 | 27435788 | T/C | 0.4073 |
| chr9: 29224171 | 9 | 29224171 | T/C | 0.4992 |
| chr9: 31886588 | 9 | 31886588 | G/A | 0.4948 |
| chr9: 33212985 | 9 | 33212985 | T/C | 0.4996 |
| chr9: 35436418 | 9 | 35436418 | C/T | 0.4844 |
| chr9: 37363016 | 9 | 37363016 | G/C | 0.4996 |
| chr9: 39057580 | 9 | 39057580 | G/A | 0.4569 |
| chr9: 41758586 | 9 | 41758586 | A/C | 0.4972 |
| chr9: 43377583 | 9 | 43377583 | C/T | 0.4561 |
| chr9: 44837991 | 9 | 44837991 | A/G | 0.4996 |
| chr9: 47413940 | 9 | 47413940 | C/T | 0.4477 |
| chr9: 49420887 | 9 | 49420887 | C/A | 0.48 |
| chr9: 51267024 | 9 | 51267024 | T/C | 0.4948 |
| chr9: 53379758 | 9 | 53379758 | G/T | 0.4952 |
| chr9: 55422398 | 9 | 55422398 | C/T | 0.4796 |
| chr9: 57258001 | 9 | 57258001 | G/A | 0.4988 |
| chr9: 59236293 | 9 | 59236293 | C/T | 0.4876 |
| chr9: 62650382 | 9 | 62650382 | C/G | 0.4872 |
| chr9: 64191131 | 9 | 64191131 | A/G | 0.478 |
| chr9: 66373455 | 9 | 66373455 | A/C | 0.4952 |
| chr9: 68330426 | 9 | 68330426 | A/C | 0.5 |
| chr9: 70113064 | 9 | 70113064 | C/A | 0.4868 |
| chr9: 72948740 | 9 | 72948740 | A/G | 0.4892 |
| chr9: 74910536 | 9 | 74910536 | C/T | 0.5 |
| chr9: 77198935 | 9 | 77198935 | A/C | 0.4976 |
| chr9: 79682260 | 9 | 79682260 | A/G | 0.4996 |
| chr9: 81526163 | 9 | 81526163 | C/T | 0.4912 |
| chr9: 100656748 | 9 | 100656748 | A/G | 0.4289 |
| chr10: 556085 | 10 | 556085 | T/A | 0.4956 |
| chr10: 1730897 | 10 | 1730897 | A/T | 0.4748 |
| ARS-BFGL-NGS-61586 | 10 | 6413192 | A/G | 0.4956 |
| BovineHD1000002399 | 10 | 7472054 | A/G | 0.4988 |
| chr10: 9153211 | 10 | 9153211 | T/G | 0.4968 |
| BovineHD1000004574 | 10 | 13579099 | C/T | 0.4445 |
| chr10: 15227385 | 10 | 15227385 | C/G | 0.4988 |
| chr10: 17193397 | 10 | 17193397 | G/C | 0.4529 |
| chr10: 19427128 | 10 | 19427128 | C/T | 0.4669 |
| chr10: 21078628 | 10 | 21078628 | C/T | 0.4928 |
| chr10: 23361431 | 10 | 23361431 | C/T | 0.49 |
| chr10: 26456276 | 10 | 26456276 | G/A | 0.4996 |
| chr10: 28994288 | 10 | 28994288 | C/G | 0.4996 |
| chr10: 30582027 | 10 | 30582027 | C/T | 0.4848 |
| chr10: 32552401 | 10 | 32552401 | C/T | 0.3966 |
| chr10: 34431386 | 10 | 34431386 | T/A | 0.4944 |
| BovineHD1000011551 | 10 | 37319439 | A/C | 0.4968 |
| chr10: 39588228 | 10 | 39588228 | A/G | 0.4804 |
| chr10: 41213432 | 10 | 41213432 | G/A | 0.4948 |
| chr10: 43406860 | 10 | 43406860 | A/C | 0.4085 |
| BovineHD1000013921 | 10 | 46503089 | A/G | 0.4976 |
| chr10: 48504197 | 10 | 48504197 | G/C | 0.47 |
| chr10: 50125360 | 10 | 50125360 | G/A | 0.4988 |
| chr10: 52159167 | 10 | 52159167 | T/G | 0.488 |
| chr10: 54236151 | 10 | 54236151 | C/T | 0.4996 |
| chr10: 56198215 | 10 | 56198215 | C/T | 0.4996 |
| chr10: 58765458 | 10 | 58765458 | C/T | 0.4625 |
| chr10: 61728710 | 10 | 61728710 | T/A | 0.4952 |
| chr10: 63028632 | 10 | 63028632 | A/G | 0.4988 |
| chr10: 66983606 | 10 | 66983606 | A/G | 0.4856 |
| chr10: 70944177 | 10 | 70944177 | C/G | 0.4988 |
| chr10: 73413069 | 10 | 73413069 | T/C | 0.4816 |
| chr10: 76359926 | 10 | 76359926 | G/C | 0.4748 |
| chr10: 78335635 | 10 | 78335635 | A/G | 0.4952 |
| chr10: 80305425 | 10 | 80305425 | C/A | 0.4477 |
| chr10: 82391519 | 10 | 82391519 | A/G | 0.4964 |
| chr10: 84072952 | 10 | 84072952 | G/A | 0.4924 |
| chr10: 86098247 | 10 | 86098247 | T/C | 0.4944 |
| chr10: 88459679 | 10 | 88459679 | G/A | 0.3854 |
| chr10: 90765222 | 10 | 90765222 | A/C | 0.492 |
| chr10: 92017959 | 10 | 92017959 | C/T | 0.496 |
| chr10: 94160653 | 10 | 94160653 | C/T | 0.4876 |
| chr10: 96254810 | 10 | 96254810 | G/A | 0.4557 |
| chr10: 98176366 | 10 | 98176366 | C/G | 0.4892 |
| chr10: 102379025 | 10 | 102379025 | G/A | 0.4996 |
| chr11: 1816232 | 11 | 1816232 | C/A | 0.4892 |
| BovineHD1100002103 | 11 | 5778054 | T/C | 0.4864 |
| chr11: 9449037 | 11 | 9449037 | C/T | 0.4984 |
| chr11: 16181304 | 11 | 16181304 | G/A | 0.397 |
| chr11: 18148960 | 11 | 18148960 | T/C | 0.4421 |
| chr11: 20898079 | 11 | 20898079 | G/A | 0.4952 |
| chr11: 23036171 | 11 | 23036171 | C/T | 0.5 |
| BovineHD1100007798 | 11 | 26080039 | C/T | 0.4964 |
| chr11: 28101895 | 11 | 28101895 | G/T | 0.4988 |
| BovineHD1100009590 | 11 | 31928844 | G/A | 0.4948 |
| chr11: 34101385 | 11 | 34101385 | C/T | 0.4677 |
| chr11: 36285809 | 11 | 36285809 | A/G | 0.5 |
| chr11: 39217910 | 11 | 39217910 | C/T | 0.4952 |
| chr11: 42411510 | 11 | 42411510 | T/C | 0.498 |
| chr11: 44850419 | 11 | 44850419 | A/G | 0.4944 |
| BovineHD1100013977 | 11 | 47686080 | G/T | 0.5 |
| chr11: 49991996 | 11 | 49991996 | A/G | 0.4976 |
| chr11: 52270391 | 11 | 52270391 | A/G | 0.4996 |
| chr11: 56101323 | 11 | 56101323 | G/C | 0.4952 |
| chr11: 58578189 | 11 | 58578189 | G/T | 0.4653 |
| chr11: 62962927 | 11 | 62962927 | C/T | 0.4888 |
| chr11: 65495916 | 11 | 65495916 | G/T | 0.4984 |
| chr11: 67558272 | 11 | 67558272 | C/T | 0.4996 |
| chr11: 69888219 | 11 | 69888219 | C/A | 0.4952 |
| chr11: 72119562 | 11 | 72119562 | C/T | 0.4844 |
| chr11: 76815091 | 11 | 76815091 | A/C | 0.4904 |
| chr11: 79430810 | 11 | 79430810 | T/C | 0.4573 |
| BovineHD1100022903 | 11 | 79980970 | T/C | 0.4992 |
| BovineHD1100023475 | 11 | 81959151 | G/T | 0.4976 |
| chr11: 83237290 | 11 | 83237290 | G/A | 0.4808 |
| chr11: 86007382 | 11 | 86007382 | G/C | 0.4856 |
| chr11: 88141810 | 11 | 88141810 | C/T | 0.4728 |
| BovineHD1100027008 | 11 | 92814391 | T/C | 0.5 |
| chr11: 94211519 | 11 | 94211519 | G/T | 0.4988 |
| chr11: 98432605 | 11 | 98432605 | C/T | 0.4964 |
| chr11: 100052622 | 11 | 100052622 | C/A | 0.498 |
| chr11: 103917923 | 11 | 103917923 | G/C | 0.4932 |
| chr12: 1319700 | 12 | 1319700 | A/G | 0.4944 |
| BovineHD1200001590 | 12 | 5423854 | G/A | 0.4884 |
| chr12: 7456083 | 12 | 7456083 | C/T | 0.4968 |
| chr12: 9274719 | 12 | 9274719 | A/G | 0.4916 |
| chr12: 11817572 | 12 | 11817572 | C/A | 0.4996 |
| chr12: 14528558 | 12 | 14528558 | G/C | 0.4928 |
| chr12: 17152561 | 12 | 17152561 | T/C | 0.4852 |
| chr12: 20418884 | 12 | 20418884 | C/T | 0.4988 |
| chr12: 23652977 | 12 | 23652977 | C/T | 0.474 |
| chr12: 24780202 | 12 | 24780202 | T/C | 0.4892 |
| chr12: 26767294 | 12 | 26767294 | T/G | 0.4992 |
| chr12: 29031933 | 12 | 29031933 | C/T | 0.4948 |
| chr12: 31074209 | 12 | 31074209 | C/A | 0.482 |
| chr12: 33937183 | 12 | 33937183 | G/C | 0.4637 |
| chr12: 35685597 | 12 | 35685597 | A/G | 0.4968 |
| chr12: 37689687 | 12 | 37689687 | T/G | 0.4944 |
| chr12: 39546498 | 12 | 39546498 | A/C | 0.4944 |
| chr12: 41364155 | 12 | 41364155 | G/T | 0.4984 |
| chr12: 44447101 | 12 | 44447101 | A/G | 0.4856 |
| chr12: 48802895 | 12 | 48802895 | C/T | 0.4932 |
| chr12: 51164014 | 12 | 51164014 | A/G | 0.4988 |
| chr12: 54348649 | 12 | 54348649 | T/C | 0.492 |
| chr12: 70218372 | 12 | 70218372 | T/C | 0.3926 |
| BovineHD1200022318 | 12 | 78387270 | A/G | 0.496 |
| BTA-30932-no-rs | 12 | 83614359 | G/A | 0.4685 |
| chr12: 87182358 | 12 | 87182358 | C/T | 0.4944 |
| chr13: 1517586 | 13 | 1517586 | C/T | 0.494 |
| BovineHD1300001203 | 13 | 4630391 | A/G | 0.4936 |
| chr13: 7362945 | 13 | 7362945 | C/T | 0.4796 |
| chr13: 9892854 | 13 | 9892854 | G/A | 0.4629 |
| chr13: 13843476 | 13 | 13843476 | A/G | 0.4932 |
| chr13: 15318011 | 13 | 15318011 | T/A | 0.4756 |
| chr13: 17297299 | 13 | 17297299 | G/A | 0.4433 |
| chr13: 19474034 | 13 | 19474034 | C/T | 0.3878 |
| chr13: 21291531 | 13 | 21291531 | C/T | 0.4972 |
| chr13: 23450925 | 13 | 23450925 | T/C | 0.4924 |
| BovineHD4100010034 | 13 | 26047739 | A/C | 0.4964 |
| chr13: 28918149 | 13 | 28918149 | C/G | 0.4046 |
| chr13: 30005237 | 13 | 30005237 | C/T | 0.4964 |
| chr13: 32728171 | 13 | 32728171 | T/C | 0.4808 |
| chr13: 36138561 | 13 | 36138561 | G/A | 0.4964 |
| chr13: 39437219 | 13 | 39437219 | G/A | 0.4996 |
| chr13: 41477551 | 13 | 41477551 | A/G | 0.4928 |
| chr13: 44551336 | 13 | 44551336 | C/G | 0.494 |
| BovineHD1300013764 | 13 | 46950685 | C/T | 0.4557 |
| BTB-01125985 | 13 | 51118474 | C/A | 0.496 |
| chr13: 58768742 | 13 | 58768742 | G/A | 0.492 |
| BovineHD1300018739 | 13 | 65998236 | A/G | 0.4868 |
| chr13: 70658985 | 13 | 70658985 | T/C | 0.4912 |
| BovineHD1300024312 | 13 | 83560269 | A/G | 0.4984 |
| chr14: 358943 | 14 | 358943 | T/C | 0.4605 |
| chr14: 3080193 | 14 | 3080193 | G/A | 0.4928 |
| chr14: 6491593 | 14 | 6491593 | T/C | 0.4545 |
| Hapmap54453-rs29015999 | 14 | 10812434 | C/T | 0.4976 |
| chr14: 12207325 | 14 | 12207325 | C/T | 0.4968 |
| BovineHD1400004049 | 14 | 14029735 | T/C | 0.4876 |
| BovineHD1400005023 | 14 | 17624295 | G/A | 0.496 |
| chr14: 19580120 | 14 | 19580120 | T/C | 0.4992 |
| chr14: 21343979 | 14 | 21343979 | A/G | 0.498 |
| chr14: 23436581 | 14 | 23436581 | G/A | 0.498 |
| chr14: 25897491 | 14 | 25897491 | G/A | 0.5 |
| chr14: 27996180 | 14 | 27996180 | C/A | 0.4972 |
| chr14: 29576356 | 14 | 29576356 | T/A | 0.4972 |
| chr14: 31898098 | 14 | 31898098 | T/C | 0.4988 |
| BovineHD1400010360 | 14 | 35973935 | T/C | 0.4573 |
| chr14: 38491622 | 14 | 38491622 | A/G | 0.4992 |
| chr14: 40835506 | 14 | 40835506 | G/A | 0.4828 |
| chr14: 42946639 | 14 | 42946639 | C/G | 0.3806 |
| chr14: 44071226 | 14 | 44071226 | A/C | 0.4976 |
| chr14: 47444785 | 14 | 47444785 | A/G | 0.4996 |
| chr14: 49311562 | 14 | 49311562 | T/C | 0.4984 |
| chr14: 51783709 | 14 | 51783709 | A/G | 0.4824 |
| BovineHD1400014971 | 14 | 53111814 | C/T | 0.488 |
| BovineHD4100011609 | 14 | 56726986 | C/T | 0.4724 |
| chr14: 58774396 | 14 | 58774396 | A/T | 0.4976 |
| chr14: 61264006 | 14 | 61264006 | C/T | 0.4896 |
| chr14: 63211114 | 14 | 63211114 | A/T | 0.4034 |
| chr14: 65283781 | 14 | 65283781 | G/A | 0.4964 |
| chr14: 68940797 | 14 | 68940797 | C/T | 0.5 |
| chr14: 73268228 | 14 | 73268228 | G/A | 0.4984 |
| BovineHD1400023889 | 14 | 84283231 | T/C | 0.4992 |
| chr15: 705104 | 15 | 705104 | A/G | 0.4996 |
| chr15: 1426673 | 15 | 1426673 | A/G | 0.486 |
| chr15: 13966063 | 15 | 13966063 | A/T | 0.4968 |
| chr15: 15353085 | 15 | 15353085 | T/C | 0.4976 |
| chr15: 17863316 | 15 | 17863316 | A/G | 0.5 |
| chr15: 20577613 | 15 | 20577613 | T/G | 0.4984 |
| chr15: 22753967 | 15 | 22753967 | G/A | 0.4996 |
| chr15: 24704857 | 15 | 24704857 | A/C | 0.4896 |
| chr15: 26010177 | 15 | 26010177 | C/T | 0.4876 |
| chr15: 28586304 | 15 | 28586304 | C/G | 0.4685 |
| chr15: 31931965 | 15 | 31931965 | T/C | 0.4996 |
| chr15: 33279400 | 15 | 33279400 | T/A | 0.4561 |
| chr15: 34120648 | 15 | 34120648 | T/G | 0.488 |
| chr15: 35353463 | 15 | 35353463 | C/A | 0.4988 |
| chr15: 36631768 | 15 | 36631768 | A/G | 0.4996 |
| chr15: 37936745 | 15 | 37936745 | T/C | 0.4988 |
| chr15: 41112170 | 15 | 41112170 | G/A | 0.4852 |
| chr15: 43478045 | 15 | 43478045 | T/C | 0.4896 |
| chr15: 45153486 | 15 | 45153486 | C/A | 0.4952 |
| chr15: 47802027 | 15 | 47802027 | C/T | 0.4856 |
| chr15: 49090962 | 15 | 49090962 | G/A | 0.4972 |
| chr15: 51607362 | 15 | 51607362 | T/C | 0.4972 |
| chr15: 53006024 | 15 | 53006024 | T/C | 0.4936 |
| BovineHD1500016013 | 15 | 55392541 | G/A | 0.4996 |
| chr15: 58357732 | 15 | 58357732 | A/G | 0.4964 |
| chr15: 61616402 | 15 | 61616402 | C/T | 0.4964 |
| chr15: 64303035 | 15 | 64303035 | A/C | 0.4505 |
| chr15: 67002416 | 15 | 67002416 | T/C | 0.4597 |
| chr15: 70085418 | 15 | 70085418 | G/A | 0.4984 |
| chr15: 72322212 | 15 | 72322212 | C/T | 0.4561 |
| chr15: 75697490 | 15 | 75697490 | A/G | 0.4984 |
| chr15: 79573827 | 15 | 79573827 | T/A | 0.4908 |
| chr15: 82767382 | 15 | 82767382 | C/T | 0.4976 |
| chr15: 84713024 | 15 | 84713024 | G/A | 0.49 |
| chr16: 77616 | 16 | 77616 | A/C | 0.4852 |
| ARS-BFGL-NGS-10856 | 16 | 1690484 | C/T | 0.4896 |
| chr16: 3834931 | 16 | 3834931 | A/G | 0.4828 |
| chr16: 5283226 | 16 | 5283226 | G/A | 0.4916 |
| chr16: 7392772 | 16 | 7392772 | C/G | 0.4968 |
| chr16: 9445946 | 16 | 9445946 | T/C | 0.4984 |
| chr16: 11735482 | 16 | 11735482 | G/C | 0.4976 |
| chr16: 12752913 | 16 | 12752913 | C/T | 0.4824 |
| chr16: 14240346 | 16 | 14240346 | C/T | 0.4936 |
| chr16: 15357072 | 16 | 15357072 | A/G | 0.4353 |
| chr16: 18018781 | 16 | 18018781 | T/C | 0.4241 |
| chr16: 19501245 | 16 | 19501245 | C/T | 0.4968 |
| chr16: 21269494 | 16 | 21269494 | G/A | 0.4221 |
| chr16: 23486555 | 16 | 23486555 | G/T | 0.4976 |
| BovineHD1600008020 | 16 | 28632394 | C/T | 0.4996 |
| chr16: 34333202 | 16 | 34333202 | A/G | 0.4844 |
| chr16: 36817430 | 16 | 36817430 | A/T | 0.4968 |
| chr16: 38789947 | 16 | 38789947 | A/G | 0.4996 |
| chr16: 41098125 | 16 | 41098125 | A/G | 0.498 |
| chr16: 44671987 | 16 | 44671987 | G/A | 0.4201 |
| chr16: 46410126 | 16 | 46410126 | C/T | 0.498 |
| chr16: 48803795 | 16 | 48803795 | G/A | 0.496 |
| chr16: 50602909 | 16 | 50602909 | A/G | 0.5 |
| BovineHD1600014931 | 16 | 53827496 | C/T | 0.3686 |
| chr16: 55295999 | 16 | 55295999 | G/A | 0.4964 |
| chr16: 57769675 | 16 | 57769675 | A/G | 0.4952 |
| chr16: 59139262 | 16 | 59139262 | G/T | 0.4896 |
| chr16: 61094760 | 16 | 61094760 | C/A | 0.4904 |
| chr16: 63552899 | 16 | 63552899 | G/A | 0.4984 |
| chr16: 65136800 | 16 | 65136800 | A/G | 0.4964 |
| chr16: 67144941 | 16 | 67144941 | C/T | 0.4984 |
| chr16: 69668766 | 16 | 69668766 | T/C | 0.4952 |
| chr16: 71320729 | 16 | 71320729 | G/A | 0.4661 |
| chr16: 73804367 | 16 | 73804367 | G/T | 0.492 |
| chr16: 75591725 | 16 | 75591725 | G/C | 0.4932 |
| chr16: 78837702 | 16 | 78837702 | G/A | 0.472 |
| chr16: 80439174 | 16 | 80439174 | C/T | 0.4868 |
| chr16: 81680738 | 16 | 81680738 | G/A | 0.3534 |
| chr17: 3949 | 17 | 3949 | A/G | 0.365 |
| BovineHD1700000599 | 17 | 2697332 | C/T | 0.4972 |
| chr17: 4148744 | 17 | 4148744 | G/C | 0.4992 |
| chr17: 6329595 | 17 | 6329595 | C/T | 0.4788 |
| chr17: 9313408 | 17 | 9313408 | C/T | 0.4904 |
| chr17: 13875510 | 17 | 13875510 | T/C | 0.4996 |
| chr17: 15091533 | 17 | 15091533 | A/G | 0.494 |
| chr17: 17047819 | 17 | 17047819 | G/A | 0.4253 |
| chr17: 19422833 | 17 | 19422833 | T/C | 0.4996 |
| chr17: 21001768 | 17 | 21001768 | C/G | 0.4996 |
| chr17: 23725618 | 17 | 23725618 | A/G | 0.4984 |
| chr17: 25823983 | 17 | 25823983 | T/C | 0.4992 |
| chr17: 28268900 | 17 | 28268900 | C/T | 0.4996 |
| chr17: 32282994 | 17 | 32282994 | G/C | 0.4904 |
| chr17: 34678792 | 17 | 34678792 | C/T | 0.4964 |
| chr17: 37147703 | 17 | 37147703 | T/A | 0.4888 |
| chr17: 39057027 | 17 | 39057027 | A/G | 0.488 |
| BovineHD1700013264 | 17 | 47362503 | A/G | 0.4996 |
| chr17: 50542329 | 17 | 50542329 | T/C | 0.4992 |
| chr17: 52756216 | 17 | 52756216 | G/A | 0.5 |
| chr17: 57107543 | 17 | 57107543 | G/T | 0.4832 |
| chr17: 60037087 | 17 | 60037087 | C/A | 0.4996 |
| chr17: 62141105 | 17 | 62141105 | T/A | 0.4964 |
| chr17: 72829287 | 17 | 72829287 | T/C | 0.4716 |
| chr18: 1103982 | 18 | 1103982 | G/A | 0.4936 |
| BovineHD1800000964 | 18 | 3336203 | G/A | 0.4988 |
| BovineHD1800001350 | 18 | 4617525 | A/C | 0.4992 |
| chr18: 5353628 | 18 | 5353628 | C/T | 0.4836 |
| chr18: 7950372 | 18 | 7950372 | A/G | 0.4984 |
| chr18: 10756875 | 18 | 10756875 | G/A | 0.4984 |
| chr18: 12449822 | 18 | 12449822 | A/C | 0.4912 |
| chr18: 14710828 | 18 | 14710828 | C/A | 0.4601 |
| chr18: 16187740 | 18 | 16187740 | G/A | 0.494 |
| chr18: 18435022 | 18 | 18435022 | G/A | 0.4968 |
| chr18: 20350656 | 18 | 20350656 | A/G | 0.4984 |
| BovineHD1800006908 | 18 | 22442470 | C/T | 0.4984 |
| chr18: 24273529 | 18 | 24273529 | A/G | 0.5 |
| chr18: 26690572 | 18 | 26690572 | A/C | 0.4724 |
| chr18: 29189137 | 18 | 29189137 | T/C | 0.4972 |
| chr18: 30525057 | 18 | 30525057 | G/A | 0.49 |
| chr18: 32419138 | 18 | 32419138 | A/G | 0.4996 |
| chr18: 35199090 | 18 | 35199090 | A/G | 0.5 |
| chr18: 36326398 | 18 | 36326398 | C/A | 0.4832 |
| chr18: 39871022 | 18 | 39871022 | A/G | 0.4968 |
| BovineHD1800012278 | 18 | 41585347 | G/A | 0.4545 |
| chr18: 44684830 | 18 | 44684830 | C/T | 0.4804 |
| chr18: 47852446 | 18 | 47852446 | A/G | 0.4944 |
| chr18: 50636269 | 18 | 50636269 | C/T | 0.4605 |
| BovineHD1900000881 | 19 | 3780300 | G/A | 0.5 |
| BovineHD1900001085 | 19 | 4588941 | A/G | 0.4996 |
| BovineHD1900002258 | 19 | 8324786 | T/C | 0.4984 |
| chr19: 9589798 | 19 | 9589798 | G/T | 0.4836 |
| chr19: 11774713 | 19 | 11774713 | T/C | 0.4972 |
| chr19: 13478080 | 19 | 13478080 | G/C | 0.4952 |
| BovineHD1900003935 | 19 | 14774749 | G/A | 0.4633 |
| chr19: 15369234 | 19 | 15369234 | A/G | 0.4712 |
| chr19: 18798193 | 19 | 18798193 | T/A | 0.4992 |
| chr19: 20993189 | 19 | 20993189 | C/T | 0.4972 |
| chr19: 22593676 | 19 | 22593676 | A/T | 0.4944 |
| chr19: 24034883 | 19 | 24034883 | T/G | 0.4661 |
| chr19: 28007039 | 19 | 28007039 | T/G | 0.4988 |
| chr19: 30734931 | 19 | 30734931 | T/G | 0.4329 |
| chr19: 33298629 | 19 | 33298629 | G/C | 0.4944 |
| chr19: 36967680 | 19 | 36967680 | A/G | 0.4968 |
| chr19: 38804144 | 19 | 38804144 | G/C | 0.492 |
| chr19: 40065445 | 19 | 40065445 | G/A | 0.4984 |
| chr19: 42493450 | 19 | 42493450 | C/T | 0.4976 |
| chr19: 44405075 | 19 | 44405075 | A/G | 0.4732 |
| chr19: 46011586 | 19 | 46011586 | G/C | 0.4816 |
| chr19: 48332465 | 19 | 48332465 | G/A | 0.4708 |
| chr19: 50508663 | 19 | 50508663 | T/C | 0.4768 |
| BovineHD1900015032 | 19 | 53717829 | C/T | 0.4968 |
| chr19: 55308139 | 19 | 55308139 | A/C | 0.4976 |
| chr19: 57917130 | 19 | 57917130 | G/T | 0.4976 |
| chr19: 59940672 | 19 | 59940672 | G/T | 0.486 |
| chr19: 62032271 | 19 | 62032271 | A/G | 0.4944 |
| chr19: 64016415 | 19 | 64016415 | A/G | 0.4525 |
| BovineHD2000000176 | 20 | 618962 | C/A | 0.5 |
| chr20: 1453145 | 20 | 1453145 | G/C | 0.4996 |
| chr20: 3233514 | 20 | 3233514 | A/C | 0.3798 |
| BovineHD2000001651 | 20 | 5217883 | A/G | 0.5 |
| Hapmap39533-BTA-108830 | 20 | 7143287 | A/C | 0.4964 |
| chr20: 9325003 | 20 | 9325003 | T/C | 0.4673 |
| chr20: 11247384 | 20 | 11247384 | A/T | 0.4984 |
| chr20: 13170776 | 20 | 13170776 | T/C | 0.4832 |
| chr20: 14423688 | 20 | 14423688 | A/G | 0.4405 |
| chr20: 16164218 | 20 | 16164218 | G/T | 0.4872 |
| chr20: 18196116 | 20 | 18196116 | G/A | 0.4996 |
| chr20: 20454853 | 20 | 20454853 | A/G | 0.4984 |
| chr20: 22542502 | 20 | 22542502 | C/T | 0.4992 |
| chr20: 24625191 | 20 | 24625191 | G/A | 0.4992 |
| chr20: 26041808 | 20 | 26041808 | G/A | 0.4832 |
| chr20: 28033599 | 20 | 28033599 | A/G | 0.4912 |
| chr20: 30619168 | 20 | 30619168 | G/A | 0.4964 |
| chr20: 32945313 | 20 | 32945313 | T/G | 0.4992 |
| BovineHD2000010338 | 20 | 36100687 | A/C | 0.4944 |
| chr20: 38864330 | 20 | 38864330 | G/A | 0.4836 |
| chr20: 40184905 | 20 | 40184905 | G/A | 0.4988 |
| chr20: 44132733 | 20 | 44132733 | C/A | 0.4872 |
| BovineHD2000016809 | 20 | 59809528 | C/T | 0.4653 |
| BovineHD2000019528 | 20 | 67358359 | A/G | 0.4996 |
| chr21: 672490 | 21 | 672490 | T/C | 0.3714 |
| BovineHD2100000817 | 21 | 4753621 | T/C | 0.4964 |
| BovineHD2100001634 | 21 | 7558076 | C/T | 0.4988 |
| ARS-BFGL-NGS-58825 | 21 | 12917017 | G/T | 0.4984 |
| BovineHD2100003468 | 21 | 13235956 | A/G | 0.488 |
| chr21: 16243319 | 21 | 16243319 | T/C | 0.4792 |
| chr21: 18091875 | 21 | 18091875 | A/G | 0.4964 |
| chr21: 19663762 | 21 | 19663762 | C/T | 0.4928 |
| chr21: 21677147 | 21 | 21677147 | C/G | 0.4932 |
| chr21: 23145931 | 21 | 23145931 | T/C | 0.4968 |
| chr21: 25634112 | 21 | 25634112 | G/A | 0.4844 |
| chr21: 27140935 | 21 | 27140935 | T/C | 0.4932 |
| chr21: 28854577 | 21 | 28854577 | A/T | 0.4952 |
| chr21: 31440243 | 21 | 31440243 | G/A | 0.4996 |
| chr21: 33089284 | 21 | 33089284 | G/A | 0.4445 |
| BovineHD2100010614 | 21 | 36212484 | G/T | 0.5 |
| chr21: 38895994 | 21 | 38895994 | T/C | 0.498 |
| chr21: 42412964 | 21 | 42412964 | T/C | 0.47 |
| chr21: 44091783 | 21 | 44091783 | T/A | 0.5 |
| chr21: 46136490 | 21 | 46136490 | C/T | 0.4996 |
| chr21: 49990739 | 21 | 49990739 | G/A | 0.4996 |
| chr21: 52183591 | 21 | 52183591 | T/A | 0.498 |
| chr21: 54577771 | 21 | 54577771 | G/C | 0.4293 |
| chr21: 56258756 | 21 | 56258756 | T/G | 0.4301 |
| chr21: 59331618 | 21 | 59331618 | T/G | 0.4792 |
| chr21: 68760593 | 21 | 68760593 | C/T | 0.4413 |
| BovineHD2200001407 | 22 | 5059795 | C/T | 0.496 |
| BovineHD2200002001 | 22 | 6637551 | T/C | 0.4305 |
| chr22: 6781996 | 22 | 6781996 | G/C | 0.4916 |
| chr22: 9048837 | 22 | 9048837 | C/T | 0.49 |
| chr22: 11789736 | 22 | 11789736 | A/G | 0.4457 |
| chr22: 14617876 | 22 | 14617876 | C/T | 0.496 |
| chr22: 16936005 | 22 | 16936005 | T/C | 0.4932 |
| ARS-BFGL-NGS-35294 | 22 | 18774368 | G/A | 0.4928 |
| chr22: 20588609 | 22 | 20588609 | T/C | 0.4633 |
| chr22: 22391024 | 22 | 22391024 | A/G | 0.5 |
| chr22: 24938859 | 22 | 24938859 | C/A | 0.4956 |
| chr22: 27440977 | 22 | 27440977 | G/A | 0.4952 |
| chr22: 31086034 | 22 | 31086034 | C/T | 0.4868 |
| chr22: 35381808 | 22 | 35381808 | C/A | 0.4972 |
| chr22: 36755505 | 22 | 36755505 | T/C | 0.478 |
| chr22: 38682942 | 22 | 38682942 | T/C | 0.4996 |
| chr22: 39367205 | 22 | 39367205 | T/C | 0.4329 |
| chr22: 41413753 | 22 | 41413753 | A/G | 0.4896 |
| chr22: 44998145 | 22 | 44998145 | T/C | 0.4996 |
| chr22: 47307004 | 22 | 47307004 | G/C | 0.5 |
| chr22: 49463695 | 22 | 49463695 | T/C | 0.4189 |
| chr22: 56554292 | 22 | 56554292 | T/C | 0.4984 |
| chr22: 61422322 | 22 | 61422322 | G/A | 0.484 |
| chr23: 737731 | 23 | 737731 | A/G | 0.4269 |
| BovineHD2300001017 | 23 | 4401397 | T/C | 0.4832 |
| BovineHD2300001206 | 23 | 5077287 | C/T | 0.4836 |
| chr23: 7313850 | 23 | 7313850 | C/A | 0.4984 |
| chr23: 9586831 | 23 | 9586831 | T/A | 0.4996 |
| chr23: 12553957 | 23 | 12553957 | G/A | 0.4996 |
| ARS-BFGL-NGS-78514 | 23 | 14313819 | C/T | 0.4908 |
| chr23: 16538832 | 23 | 16538832 | A/G | 0.4948 |
| chr23: 18262499 | 23 | 18262499 | C/T | 0.4772 |
| chr23: 20970270 | 23 | 20970270 | A/G | 0.4904 |
| chr23: 22621637 | 23 | 22621637 | A/G | 0.4932 |
| chr23: 24292015 | 23 | 24292015 | G/T | 0.4533 |
| chr23: 25670252 | 23 | 25670252 | G/A | 0.3854 |
| chr23: 27655408 | 23 | 27655408 | T/G | 0.4944 |
| chr23: 30553540 | 23 | 30553540 | T/C | 0.4944 |
| chr23: 32360632 | 23 | 32360632 | T/C | 0.4625 |
| chr23: 34492864 | 23 | 34492864 | A/T | 0.494 |
| chr23: 36557706 | 23 | 36557706 | T/C | 0.4848 |
| chr23: 39647590 | 23 | 39647590 | C/T | 0.4896 |
| chr23: 41416362 | 23 | 41416362 | A/G | 0.4796 |
| chr23: 43316240 | 23 | 43316240 | G/A | 0.4213 |
| chr23: 45422704 | 23 | 45422704 | C/T | 0.4992 |
| chr23: 47896708 | 23 | 47896708 | C/T | 0.4992 |
| chr23: 50385911 | 23 | 50385911 | G/T | 0.4317 |
| chr23: 52503653 | 23 | 52503653 | G/A | 0.4429 |
| chr24: 1498986 | 24 | 1498986 | C/T | 0.4932 |
| BovineHD2400001782 | 24 | 6635248 | T/C | 0.4908 |
| BovineHD2400002974 | 24 | 10461518 | T/G | 0.4692 |
| BovineHD2400003128 | 24 | 11043284 | G/T | 0.4776 |
| chr24: 12213557 | 24 | 12213557 | G/A | 0.4984 |
| chr24: 14917351 | 24 | 14917351 | C/A | 0.4992 |
| chr24: 17169312 | 24 | 17169312 | T/C | 0.4996 |
| chr24: 19292120 | 24 | 19292120 | G/C | 0.498 |
| chr24: 21820599 | 24 | 21820599 | T/C | 0.474 |
| chr24: 23901873 | 24 | 23901873 | T/C | 0.4888 |
| chr24: 26177933 | 24 | 26177933 | C/T | 0.4201 |
| chr24: 27712396 | 24 | 27712396 | T/A | 0.4545 |
| chr24: 30518675 | 24 | 30518675 | C/G | 0.4972 |
| chr24: 34261251 | 24 | 34261251 | C/T | 0.4728 |
| chr24: 36738426 | 24 | 36738426 | C/A | 0.4996 |
| chr24: 39303552 | 24 | 39303552 | G/A | 0.498 |
| chr24: 42418587 | 24 | 42418587 | T/A | 0.4856 |
| chr24: 44987204 | 24 | 44987204 | T/C | 0.4904 |
| chr24: 47032284 | 24 | 47032284 | A/C | 0.5 |
| chr24: 49816564 | 24 | 49816564 | C/A | 0.4984 |
| BovineHD2400014878 | 24 | 52567761 | T/C | 0.498 |
| chr24: 54141039 | 24 | 54141039 | T/G | 0.4868 |
| chr24: 57719811 | 24 | 57719811 | A/G | 0.4513 |
| chr24: 59845272 | 24 | 59845272 | C/T | 0.4832 |
| chr24: 62616509 | 24 | 62616509 | T/C | 0.4988 |
| BovineHD2500000375 | 25 | 1817313 | C/T | 0.4824 |
| chr25: 2270319 | 25 | 2270319 | C/A | 0.4904 |
| BovineHD2500000754 | 25 | 3526794 | A/C | 0.4996 |
| chr25: 5235133 | 25 | 5235133 | A/G | 0.4848 |
| chr25: 7435898 | 25 | 7435898 | C/A | 0.4988 |
| chr25: 9485224 | 25 | 9485224 | C/T | 0.4856 |
| chr25: 11983738 | 25 | 11983738 | A/G | 0.4972 |
| chr25: 14309813 | 25 | 14309813 | A/G | 0.4888 |
| chr25: 16824007 | 25 | 16824007 | G/C | 0.498 |
| chr25: 20265328 | 25 | 20265328 | A/T | 0.498 |
| chr25: 23776686 | 25 | 23776686 | A/T | 0.5 |
| chr25: 25356383 | 25 | 25356383 | G/A | 0.476 |
| BovineHD2500007701 | 25 | 27570361 | A/G | 0.4988 |
| BovineHD2500008282 | 25 | 29754420 | A/G | 0.4996 |
| chr25: 31426099 | 25 | 31426099 | C/T | 0.4932 |
| chr25: 33447705 | 25 | 33447705 | G/A | 0.4992 |
| chr25: 35354192 | 25 | 35354192 | G/A | 0.4928 |
| chr25: 37706882 | 25 | 37706882 | C/T | 0.4948 |
| chr25: 39617743 | 25 | 39617743 | T/C | 0.4996 |
| chr25: 41421831 | 25 | 41421831 | G/C | 0.4816 |
| BovineHD2600000537 | 26 | 3159275 | A/G | 0.4976 |
| BovineHD2600001618 | 26 | 7064821 | T/G | 0.4764 |
| chr26: 8295838 | 26 | 8295838 | T/A | 0.4988 |
| chr26: 10496908 | 26 | 10496908 | A/G | 0.492 |
| chr26: 12492633 | 26 | 12492633 | G/C | 0.4968 |
| chr26: 15552507 | 26 | 15552507 | A/T | 0.4637 |
| chr26: 18648652 | 26 | 18648652 | G/A | 0.4984 |
| chr26: 20477934 | 26 | 20477934 | G/A | 0.4996 |
| chr26: 23396729 | 26 | 23396729 | C/T | 0.5 |
| chr26: 27384764 | 26 | 27384764 | A/C | 0.4924 |
| chr26: 30073565 | 26 | 30073565 | A/G | 0.4936 |
| chr26: 32876281 | 26 | 32876281 | A/T | 0.4992 |
| chr26: 36469097 | 26 | 36469097 | G/A | 0.4996 |
| BovineHD2600010617 | 26 | 38760186 | A/G | 0.5 |
| chr26: 40450911 | 26 | 40450911 | T/G | 0.5 |
| chr26: 44726125 | 26 | 44726125 | T/C | 0.5 |
| chr26: 46922819 | 26 | 46922819 | C/T | 0.4976 |
| chr26: 50226218 | 26 | 50226218 | G/A | 0.5 |
| chr27: 103736 | 27 | 103736 | C/T | 0.4908 |
| chr27: 1101354 | 27 | 1101354 | A/G | 0.4916 |
| chr27: 3226775 | 27 | 3226775 | C/A | 0.4764 |
| chr27: 5017483 | 27 | 5017483 | T/C | 0.4992 |
| BovineHD2700002543 | 27 | 8093753 | A/C | 0.4972 |
| chr27: 8768007 | 27 | 8768007 | T/A | 0.4796 |
| BovineHD2700003358 | 27 | 11305871 | T/C | 0.4988 |
| chr27: 12767506 | 27 | 12767506 | T/C | 0.4992 |
| chr27: 14747630 | 27 | 14747630 | T/A | 0.4752 |
| chr27: 16188798 | 27 | 16188798 | G/A | 0.4888 |
| chr27: 18631002 | 27 | 18631002 | G/A | 0.4988 |
| chr27: 20739697 | 27 | 20739697 | A/T | 0.4764 |
| chr27: 23238215 | 27 | 23238215 | G/A | 0.496 |
| chr27: 25163977 | 27 | 25163977 | A/T | 0.4081 |
| chr27: 27780471 | 27 | 27780471 | A/T | 0.4936 |
| chr27: 29007650 | 27 | 29007650 | C/T | 0.4976 |
| chr27: 32882318 | 27 | 32882318 | G/C | 0.4992 |
| chr27: 34293543 | 27 | 34293543 | C/T | 0.4609 |
| chr27: 37259753 | 27 | 37259753 | T/C | 0.4908 |
| chr27: 39274159 | 27 | 39274159 | C/T | 0.4944 |
| chr27: 41321218 | 27 | 41321218 | A/C | 0.4968 |
| chr28: 140159 | 28 | 140159 | T/A | 0.4812 |
| chr28: 2239999 | 28 | 2239999 | C/T | 0.4996 |
| chr28: 4180876 | 28 | 4180876 | C/G | 0.4984 |
| BovineHD2800001838 | 28 | 6164460 | G/A | 0.4728 |
| chr28: 8465913 | 28 | 8465913 | T/C | 0.498 |
| BovineHD4100018502 | 28 | 9663509 | C/T | 0.4976 |
| BovineHD2800003355 | 28 | 11042073 | T/C | 0.4393 |
| chr28: 13676267 | 28 | 13676267 | T/C | 0.4996 |
| BovineHD2800004610 | 28 | 16557050 | G/A | 0.4884 |
| chr28: 18341653 | 28 | 18341653 | T/A | 0.4752 |
| chr28: 20964219 | 28 | 20964219 | G/T | 0.5 |
| chr28: 22247295 | 28 | 22247295 | A/T | 0.496 |
| chr28: 25042637 | 28 | 25042637 | A/C | 0.4852 |
| chr28: 28278314 | 28 | 28278314 | G/A | 0.4956 |
| chr28: 30097594 | 28 | 30097594 | T/A | 0.4988 |
| chr28: 32561401 | 28 | 32561401 | G/A | 0.4952 |
| BovineHD2800009155 | 28 | 34089913 | C/A | 0.4888 |
| chr28: 36080441 | 28 | 36080441 | T/C | 0.4956 |
| chr28: 38019304 | 28 | 38019304 | C/T | 0.4992 |
| chr28: 40659487 | 28 | 40659487 | G/C | 0.4996 |
| chr28: 42821390 | 28 | 42821390 | C/T | 0.4984 |
| chr28: 44511041 | 28 | 44511041 | C/G | 0.4988 |
| chr28: 45635975 | 28 | 45635975 | C/T | 0.4992 |
| chr29: 1772654 | 29 | 1772654 | A/G | 0.4836 |
| chr29: 3970906 | 29 | 3970906 | G/A | 0.498 |
| BovineHD2900002113 | 29 | 7484023 | A/C | 0.4653 |
| chr29: 9604215 | 29 | 9604215 | G/C | 0.4968 |
| chr29: 11737448 | 29 | 11737448 | A/G | 0.4968 |
| chr29: 14120989 | 29 | 14120989 | A/G | 0.4968 |
| BovineHD2900004975 | 29 | 16675393 | G/A | 0.486 |
| chr29: 19524544 | 29 | 19524544 | T/G | 0.4976 |
| chr29: 21310589 | 29 | 21310589 | C/G | 0.4864 |
| chr29: 23512363 | 29 | 23512363 | A/C | 0.4992 |
| chr29: 25651720 | 29 | 25651720 | A/G | 0.4996 |
| chr29: 29237021 | 29 | 29237021 | C/T | 0.4932 |
| chr29: 31663327 | 29 | 31663327 | A/G | 0.4481 |
| chr29: 34085155 | 29 | 34085155 | G/A | 0.4996 |
| chr29: 36484686 | 29 | 36484686 | C/A | 0.4984 |
| chr29: 39714340 | 29 | 39714340 | A/T | 0.4844 |
| chr29: 43321959 | 29 | 43321959 | A/G | 0.4988 |
| chr29: 46568838 | 29 | 46568838 | T/G | 0.4988 |
| chr29: 48902564 | 29 | 48902564 | G/A | 0.4888 |
| BovineHD2900014811 | 29 | 50783100 | G/A | 0.4976 |

2. The SNP molecular marker combination according to claim 1, wherein Bos_taurus_UMD_3.1 serves as a reference genome for a site of each of the SNP molecular markers.

3. A probe for identifying the SNP molecular marker combination according to claim 1.

4. A gene chip prepared based on the SNP molecular marker combination according to claim 1.

5. The gene chip according to claim 4, comprising a liquid chip.

6. (canceled)

7. (canceled)

8. A method for genetic relationship identification of a Huaxi cattle, comprising the following steps: identifying a genotype of the SNP molecular marker combination according to claim 1 using a genomic DNA of a Huaxi cattle to be identified as a template; calculating an logarithm of the odds (LOD) value of a paternity index according to the genotype of the Huaxi cattle to be identified; and identifying a genetic relationship of the Huaxi cattle to be identified based on the LOD value.

9. The method according to claim 8, wherein when the LOD value is greater than 0, a candidate parent has a possibility of being a true parent, and an individual with a maximum LOD value is regarded as a most similar parent; and when the LOD value is less than 0, the candidate parent does not have the possibility of being the true parent.

10. A probe for identifying the SNP molecular marker combination according to claim 2.

11. A gene chip prepared based on the SNP molecular marker combination according to claim 2.

12. The gene chip according to claim 11, comprising a liquid chip.

13. A method for genetic relationship identification of a Huaxi cattle using the SNP molecular marker combination according to claim 1.

14. A method for genetic relationship identification of a Huaxi cattle using the SNP molecular marker combination according to claim 2.

15. A method for genetic relationship identification of a Huaxi cattle using the probe according to claim 3.

16. A method for genetic relationship identification of a Huaxi cattle using the probe according to claim 10.

17. A method for genetic relationship identification of a Huaxi cattle using the gene chip according to claim 4.

18. A method for genetic relationship identification of a Huaxi cattle using the gene chip according to claim 11.

19. A method for genetic relationship identification of a Huaxi cattle using the gene chip according to claim 5.

20. A method for genetic relationship identification of a Huaxi cattle using the gene chip according to claim 12.

21. A method for improved genetic breeding of a Huaxi cattle using the SNP molecular marker combination according to claim 1.

22. A method for improved genetic breeding of a Huaxi cattle using the SNP molecular marker combination according to claim 2.
